(19) United States
(12) Patent Application Publication
(10) Pub. No.: US 2005/0158382 A1
(43) Pub. Date: Jul. 21, 2005

(54) Controlled release formulations of opioid and nonopioid analgesics

(76) Inventors: Evangeline Cruz, Hayward, CA (US); Atul D. Ayer, Palo Alto, CA (US); Brenda J. Pollock, Cupertino, CA (US); Carmelita Garcia, Newark, CA (US); Sherry Li, Cupertino, CA (US); Alfredo M. Wong, Sunnyvale, CA (US); Lawrence G. Hamel, Mountain View, CA (US); Cheri Enders Klein, Northbrook, IL (US); Yihong Qiu, Vernon Hills, IL (US); Ye Huang, Gurnee, IL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/949,141

(22) Filed: Sep. 24, 2004

Related U.S. Application Data
(60) Provisional application No. 60/571,238, filed on May 14, 2004.
(60) Provisional application No. 60/506,195, filed on Sep. 26, 2003.

(51) Int. Cl. A61K 31/485; A61K 31/16; A61K 9/22

(52) U.S. Cl. 424/468; 514/282; 514/629

(57) ABSTRACT

Sustained release dosage forms for twice daily oral dosing to a human patient for providing relief from pain are provided. The sustained release dosage form comprises an immediate release component and a sustained release component, wherein the immediate release component and the sustained release component collectively contain a therapeutically effective amount of an opioid analgesic and a therapeutically effective amount of nonopioid analgesic. In a preferred embodiment, the nonopioid analgesic is acetaminophen and the opioid analgesic is hydrocodone and pharmaceutically acceptable salts thereof, and in preferred embodiments, the pharmaceutically acceptable salt is bitartrate. The dosage forms produce plasma profiles in a patient characterized by a Cmax for hydrocodone of between about 0.6 ng/mL/mg to about 1.4 ng/mL/mg and an AUC for hydrocodone of between about 9.1 ng*hr/mL/mg to about 19.9 ng*hr/mL/mg (per mg hydrocodone bitartrate administered) and a Cmax for acetaminophen of between about 2.8 ng/mL/mg and 7.9 ng/mL/mg and an AUC for acetaminophen of between about 28.6 ng*hr/mL/mg and about 59.1 ng*hr/mL/mg (per mg acetaminophen administered) after a single dose.

### CROSS-REFERENCE TO RELATED APPLICATION

This application claims the benefit of provisional application U.S. Ser. Nos. 60/571,238 filed May 14, 2004, and 60/506,195 filed Sep. 26, 2003, both of which are incorporated by reference herein.

### FIELD OF THE INVENTION

This invention relates generally to solid dosage forms for administering pharmaceutical agents, methods for preparing the dosage forms, and methods for providing therapeutic agents to patients in need thereof, and the like.

### BACKGROUND OF THE INVENTION

Controlled release dosage forms for delivering analgesic agents such as opioid analgesics are known in the art. Combination products providing delivery of relatively soluble drugs such as opioid analgesics and relatively insoluble drugs such as certain nonopioid analgesics are more difficult to prepare, however the preparation of some dosage forms has been reported. For example, U.S. Pat. No. 6,245,357 discloses a dosage form to deliver an opioid analgesic such as hydromorphone or morphine in combination with a nonopioid analgesic such as acetaminophen or ibuprofen, and a pharmaceutically acceptable polymer hydrogel (maltodextrin, polyalkylene oxide, polyethylene oxide, carboxyalkylcellulose), which exhibits an osmotic pressure gradient across the bilayer interior wall and exterior wall thereby imbibing fluid into the drug compartment to form a solution or a suspension comprising the drug that is hydrodynamically and osmotically delivered through a passageway from the dosage form. This patent describes the importance of the interior wall in regulating and controlling the flow of water into the dosage form, its modulation over time as pore forming agents are eluted out of the interior wall, and its ability to compensate for loss in osmotic driving force later in the delivery period. The patent also discloses a method for administering a unit dose of opioid analgesic by administering a dose of 2 mg to 8 mg for from zero to 18 hours, and 0-2 mg for from 18-24 hours. However, the dosage forms described are suitable for and intended for once a day administration, not twice a day administration, since the dosage forms deliver opioid and nonopioid analgesics over a period of 18 to 24 hours.

U.S. Pat. No. 6,284,274 describes a bilayer tablet containing an opiate analgesic, a polyalkylene oxide, polyvinylpyrrolidone and a lubricant in the first layer and a second osmotic push layer containing polyethylene oxide or carboxymethylcellulose. A bilayer tablet is also described having a non-opiate analgesic in the first layer with polyethylene oxide, polyvinylpyrrolidone and a nonionic surfactant, including polyoxyethylene fatty alcohol esters, sorbitan fatty acid esters, polyoxyethylene sorbitan fatty acid esters, polyoxyethylene sorbitan monolaurate, polyoxyethylene sorbitan monostearate, polyoxyethylene sorbitan monooleate, polyoxyethylene sorbitan monopalmitate and polyoxyethylene sorbitan monolaurylsulphate. However, the opiate and non-opiate analgesics were not combined in the bilayer tablets.

U.S. patent application Publication No. 2003/0092724 to Kao describes sustained release dosage forms in which a nonopioid analgesic and opioid analgesic are combined in a sustained release layer and in an immediate release layer. High loading of the nonopioid analgesic was achieved only in the immediate release layer. In addition, this application teaches that the relative release rates of the active agents need not be proportional to each other. Finally, the dosage forms did not release 90% of the analgesic agents within the time period reported for the dissolution profile, resulting in high amounts of residual drug in the formulations.

The family of patents represented by U.S. Pat. No. 6,387,404 to Oshlack describes dosage forms containing an immediate release core coated with a hydrophobic coating that provides sustained release. The immediate release core contains a combination of an insoluble therapeutically active agent such as acetaminophen and a soluble therapeutically active agent such as an opioid analgesic in a sustained release dosage form. The release rate of the codeine was about twice the release rate of the acetaminophen.

Additional dosage forms have been described for delivering opioid analgesics. For example, U.S. Pat. No. 5,948,787 describes morphine compositions and methods for administering morphine, and analgesic compositions comprising an opioid analgesic (including hydrocodone) a polyalkylene oxide, PVP, and a nonionic surfactant.

U.S. Pat. No. 6,491,945 describes compositions comprising hydrocodone, carboxymethylcellulose, hydroxypropylalkylcellulose, and a lubricant, optionally comprising polyvinylpyrrolidone or sorbitol.

U.S. Pat. No. 5,866,161 describes a method for administering hydrocodone using a sustained delivery bilayer comprising hydrocodone, a polyalkylene oxide, a hydroxyalkylcellulose, and a lubricant, where the hydrocodone is delivered at a controlled rate of 0.5 mg to 10 mg per hour over a period of 30 hours.

U.S. patent application Publication No. 20030077320 describes a dosage form containing both polyalkylene oxide and hydroxyalkylcellulose or alkali carboxymethylcellulose and hydroxypropylalkylcellulose, and methods of delivery over a period of 20 and 30 hours.

U.S. Pat. No.5,866,164 describes a composition having an opioid analgesic in a first layer and an opioid antagonist in a second layer.

U.S. Pat. No.5,593,695 describes morphine compositions and a method for administering morphine.

U.S. Pat. No.5,529,787 describes compositions and methods for administering hydromorphone using a bilayer composition comprising carboxymethylcellulose, polyvinylpyrrolidone and a lubricant in the drug layer and a polyalkylene oxide, osmagent, hydroxyalkylcellulose and a lubricant.

U.S. Pat. No.5,702,725 describes bilayer compositions comprising hydromorphone and methods of administering hydromorphone, comprising a polyalkylene oxide, polyvinylpyrrolidone, lubricant and a push layer.

U.S. Pat. No.5,914,131 describes dosage forms comprising hydromorphone, a method for producing hydromorphone therapy and a method for providing a hydromorphone plasma concentration. Specific dosage forms are described, with the drug layer comprising a polyalkylene oxide, polyvinylpyrrolidone, a lubricant and a push layer. Hydromorphone is delivered at a release rate of 55-85% in 1-14 hours, and 80-100% in 0-24 hours.

U.S. Pat. No.5,460,826 describes dosage forms comprising morphine and methods of administering morphine, comprising a drug composition layer comprising morphine, a polyalkylene oxide, polyvinylpyrrolidone, lubricant and a push layer.

U.S. patent application Publication No. 2003/0224051 describes controlled release dosage forms for once a day administration of oxycodone.

WO 03/092648 describes a dosage form for once a day controlled delivery of oxycodone, wherein the compound is released at a uniform rate such that the average hourly release rate from the core varies positively or negatively by no more than about 10%, 25% or 30% from either the preceding or the subsequent average hourly release rate, providing a mean steady state plasma concentration profile over a 24 hour period.

WO 03/101384 discloses a controlled release oral dosage form for once a day administration of oxycodone.

WO 01/032148 describes formulations described as suitable for twice a day administration of hydrocodone.

In none of the methods mentioned above are high load dosage forms described that are capable of providing sustained release of both acetaminophen and hydrocodone at proportional rates to a patient in need of treatment for twice daily administration.

### SUMMARY OF THE INVENTION

Accordingly, it is a primary object of the invention to address the aforementioned need in the art by providing novel methods and dosage forms for drug delivery using sustained release dosage forms for administering opioid analgesics and nonopioid analgesics over a sustained period of time.

It is an object of the present invention to provide bioavailable formulations of an opioid and nonopioid analgesic, and in particular, hydrocodone and acetaminophen, that provide analgesia using less frequent dosing than available using immediate release formulations.

It is a further object of the present invention to provide an orally administered pharmaceutical dosage form of hydrocodone and acetaminophen that is suitable for twice-a-day administration. It is a further object of the present invention to provide oral dosage forms of hydrocodone and acetaminophen, or a pharmaceutically acceptable salt thereof, which are administrable on a twice-a-day basis and which provide effective treatment of pain in mammals, and in particular, humans.

It is a further object of the invention to control moderate to severe pain in patients who require around-the-clock opioid medications for more than a few days by administering a formulation of hydrocodone and acetaminophen providing pharmacokinetic parameters consistent with twice daily dosing.

It is a further object of the invention to provide twice daily dosing of an analgesic dosage form containing an opioid and nonopioid analgesic, and hydrocodone and acetaminophen in particular, in order to reduce the risk of missed doses, thereby decreasing the frequency and severity of breakthrough pain and minimizing a source of patient anxiety and providing an improved quality of life.

It is a further object of the invention to provide patients with a treatment for their pain which provides sufficient plasma levels of opioid and nonopioid analgesic to provide a reduction in pain intensity within about 1 hour after administration, and which treatment further provides sufficient plasma levels of opioid and nonopioid analgesic to provide pain relief at a later time in the dosage interval at which it may be expected that patients may experience breakthrough pain.

It is a further object of the invention to provide a twice-a-day controlled release dosage form providing plasma concentration profiles exhibiting a two component delivery characterized by a relatively rapid, initial rise in plasma levels of opioid and nonopioid analgesic (e.g, hydrocodone and acetaminophen), as demonstrated by reduced pain within about 1 hour after administration, followed by a prolonged delivery providing therapeutically effective levels of opioid and nonopioid analgesic in plasma, providing pain relief both early and during the 12 hour dosing period.

It is a further object of the invention to accomplish the above objects utilizing a controlled release formulation of hydrocodone and acetaminophen, which when administered every 12 hours, provides plasma concentrations that are relatively equivalent to a similar dose of immediate-release hydrocodone and acetaminophen dosed every 4 hours.

It is a further object of the invention to provide a sustained release formulation of hydrocodone and acetaminophen which, when administered every 12 hours, provides a lower maximum and higher minimum plasma hydrocodone and acetaminophen concentrations (e.g., a smaller peak to trough fluctuation) than those from the same total dose of immediate-release hydrocodone and acetaminophen administered every 4 hours.

In view of the above objects and others, the present invention in certain embodiments is directed to a solid sustained release twice-a-day oral dosage form of an opioid analgesic and nonopioid analgesic, in particular, hydrocodone and acetaminophen, which provides sustained release of each of said opioid analgesic and said nonopioid analgesic at rates proportional to their respective amounts in said dosage form when administered to a patient. Preferably, administration of the dosage form results in a rapid rise in plasma concentration which occurs early in the dosage interval, such that the patient experiences a reduced pain intensity within about 1 hour after administration, and further provides sufficient plasma concentrations of hydrocodone and acetaminophen to provide pain relief later during the dosage interval when patients might anticipate breakthrough pain.

Sustained release dosage forms for twice daily oral dosing to a human patient for providing relief from pain are provided. The sustained release dosage form comprises an immediate release component and a sustained release component, wherein the immediate release component and the sustained release component collectively contain a therapeutically effective amount of an opioid analgesic and a therapeutically effective amount of nonopioid analgesic, wherein the amount of nonopioid analgesic is between about 20 and about 100 times the amount of opioid analgesic by weight, and the sustained release component provides sustained release of each of the opioid analgesic and the nonopioid analgesic at rates proportional to each other. In certain embodiments, the amount of nonopioid analgesic is between about 20 and about 40 times the amount of opioid analgesic by weight. In particular embodiments, the amount of nonopioid analgesic is between about 27 and about 34 times the amount of opioid analgesic by weight. In a preferred embodiment, the nonopioid analgesic is acetaminophen and the opioid analgesic is hydrocodone bitartrate. In certain embodiments, the dosage form contains a loading of acetaminophen of at least 60% by weight, and more typically of between about 75% and about 95% by weight.

In another embodiment, the sustained release dosage form comprises an analgesic composition comprising a therapeutically effective amount of a nonopioid analgesic and an opioid analgesic; a means for providing an initial release of the nonopioid analgesic and opioid analgesic sufficient to provide an initial peak concentration in the plasma of the human patient, and a means for providing a second release sustained for up to about 12 hours to provide sustained plasma concentrations of the nonopioid analgesic and opioid analgesic sufficient to provide sustained relief from pain for about 12 hours, wherein said means further provides for proportional release of the nonopioid analgesic and opioid analgesic.

In another embodiment, a controlled release dosage form is provided which is suitable for twice daily oral administration to a human patient for effective relief from pain, comprising: an analgesic composition comprising a therapeutically effective amount of a nonopioid analgesic and an opioid analgesic in a relative weight ratio between about 27 and about 34; and a mechanism providing controlled release of the nonopioid analgesic and opioid analgesic. Preferably, the release rates of the nonopioid analgesic and opioid analgesic are proportional to each other.

In yet another embodiment, a bilayer dosage form of an opioid analgesic and a nonopioid analgesic is provided for twice daily oral administration to a human patient, comprising a drug layer comprising a therapeutically effective amount of the opioid analgesic and nonopioid analgesic, a nondrug layer comprising a high molecular weight polymer providing sustained release of the opioid analgesic and the nonopioid analgesic as an erodible composition upon imbibition of water, a semipermeable membrane providing a controlled rate of entry of water into the dosage form, and a flow promoting layer located between the drug layer and the semipermeable membrane.

In another embodiment, a sustained release dosage form is provided for twice daily oral administration comprising a drug composition containing a high load of a relatively insoluble nonopioid analgesic and a smaller amount of a relatively soluble opioid analgesic, an expandable composition that expands on imbibing water present in the environment of use, and a rate controlling membrane moderating the rate at which the expandable composition imbibes water, wherein said sustained release dosage form provides for proportional release of said nonopioid analgesic and said opioid analgesic over an extended period of time.

In a preferred embodiment, the dosage form comprises: (1) a semipermeable wall defining a cavity and including an exit orifice formed or formable therein; (2) a drug layer comprising a therapeutically effective amount of an opioid analgesic and a nonopioid analgesic contained within the cavity and located adjacent to the exit orifice; (3) a push displacement layer contained within the cavity and located distal from the exit orifice; (4) a flow-promoting layer between the inner surface of the semipermeable wall and at least the external surface of the drug layer that is opposite the wall; and the dosage form provides an in vitro rate of release of the opioid analgesic and the nonopioid analgesic for up to about 12 hours after being contacted with water in the environment of use. Preferably, the drug layer contains a loading of the nonopioid analgesic of at least 60% by weight, and in certain embodiments, the drug layer contains a loading of the nonopioid analgesic of between about 75% and about 95% by weight, and in other embodiments, the drug layer contains a loading of the nonopioid analgesic between about 80% and about 85% by weight.

Preferably the drug layer contains a loading of the opioid analgesic between about 1% and about 10% by weight, and in certain embodiments, the drug layer contains a loading of the opioid analgesic between about 2% and about 6% by weight. The amount of the nonopioid analgesic is generally between about 20 and about 100, more typically between about 20 and about 40 times the amount of the opioid analgesic by weight, or most typically, the amount of the nonopioid analgesic is between about 27 and about 34 times the amount of the opioid analgesic by weight.

Preferably, the dosage form releases the opioid analgesic and the nonopioid analgesic at rates proportional to each other, and the drug layer is exposed to the environment of use as an erodible composition. The in vitro rate of release of the opioid analgesic and the nonopioid analgesic is zero order or ascending. In certain embodiments, the in vitro rate of release of the opioid analgesic and nonopioid analgesic is maintained for from about 6 hours to about 10 hours, and in a preferred embodiment, the in vitro rate of release of the opioid analgesic and nonopioid analgesic is maintained for about 8 hours.

In additional embodiments, the dosage form further comprises a drug coating comprising a therapeutically effective amount of an opioid analgesic and nonopioid analgesic sufficient to provide an analgesic effect in a patient in need thereof. The drug coating can comprise from about 60% to about 96.99% acetaminophen by weight, and more typically, the drug coating comprises from about 75% to about 89.5% acetaminophen by weight. The drug coating can comprise from about 0.01% to about 25% hydrocodone bitartrate by weight, more preferably from about 0.5% to about 15% hydrocodone bitartrate by weight, even more preferably from about 1% to about 3% hydrocodone bitartrate by weight.

In particular embodiments, the sustained release dosage form exhibits a release rate in vitro of the opioid analgesic and nonopioid analgesic of from about 19% to about 49% released after 0.75 hour, from about 40% to about 70% released after 3 hours, and at least about 80% released after 6 hours. In additional embodiments, the dosage form exhibits a release rate in vitro of the opioid analgesic and nonopioid analgesic of from about 19% to about 49% released after 0.75 hour, from about 35% to about 65% released after 3 hours, and at least about 80% released after 8 hours. In yet other embodiments, the dosage form exhibits a release rate in vitro of the opioid analgesic and nonopioid analgesic of from about 19% to about 49% released after 0.75 hour, from about 35% to about 65% released after 4 hours, and at least about 80% released after 10 hours.

In certain embodiments, the opioid analgesic is selected from hydrocodone, hydromorphone, oxymorphone, methadone, morphine, codeine, or oxycodone, or pharmaceutically acceptable salts thereof, and the nonopioid analgesic is preferably acetaminophen. In a preferred embodiment, the nonopioid analgesic is acetaminophen and the opioid analgesic is hydrocodone bitartrate.

In another embodiment, methods of using the dosage forms are described. Methods for providing an effective concentration of an opioid analgesic and nonopioid analgesic in the plasma of a human patient for the treatment of pain, methods for treating pain in a human patient, methods for providing sustained release of a nonopioid analgesic and opioid analgesic, and methods for providing an effective amount of an analgesic composition for treating pain in a human patient in need thereof are provided. In one embodiment, the methods comprise orally administering to a human patient a sustained release dosage form comprising an immediate release component and a sustained release component, wherein the immediate release component and the sustained release component collectively contain a therapeutically effective amount of an opioid analgesic and a therapeutically effective amount of nonopioid analgesic, wherein the amount of nonopioid analgesic is between about 20 and about 100 times the amount of opioid analgesic by weight, and the sustained release component provides sustained release of each of the opioid analgesic and the nonopioid analgesic at rates proportional to each other. In particular embodiments, the amount of nonopioid analgesic is between about 20 and about 40 times the amount of opioid analgesic, and in additional embodiments, the amount of nonopioid analgesic is between about 27 and about 34 times the amount of opioid analgesic by weight. In a preferred embodiment, the nonopioid analgesic is acetaminophen and the opioid analgesic is hydrocodone bitartrate. In certain embodiments, the dosage form contains a loading of acetaminophen of at least 60% by weight, and more typically of between about 75% and about 95% by weight.

In another embodiment, the methods comprise orally administering a sustained release dosage form comprising an analgesic composition comprising a therapeutically effective amount of a nonopioid analgesic and an opioid analgesic; a means for providing an initial release of the nonopioid analgesic and opioid analgesic sufficient to provide an initial peak concentration in the plasma of the human patient, and a means for providing a second release sustained for up to about 12 hours to provide sustained plasma concentrations of the nonopioid analgesic and opioid analgesic sufficient to provide sustained relief from pain for about 12 hours, wherein said means further provides for proportional release of the nonopioid analgesic and opioid analgesic.

In another embodiment, the methods comprise orally administering a controlled release dosage form suitable for twice daily oral administration to a human patient for effective relief from pain, comprising: an analgesic composition comprising a therapeutically effective amount of a nonopioid analgesic and an opioid analgesic in a relative weight ratio between about 20 and about 40, or between about 27 and about 34; and a mechanism providing controlled release of the nonopioid analgesic and opioid analgesic, wherein the release rates of the nonopioid analgesic and opioid analgesic are proportional to each other.

In yet another embodiment, the methods comprise orally administering a bilayer dosage form of an opioid analgesic and a nonopioid analgesic suitable for twice daily oral administration to a human patient, comprising a drug layer comprising a therapeutically effective amount of the opioid analgesic and nonopioid analgesic, a nondrug layer comprising a high molecular weight polymer providing sustained release of the opioid analgesic and the nonopioid analgesic as an erodible composition upon imbibition of water, a semipermeable membrane providing a controlled rate of entry of water into the dosage form, and a flow promoting layer located between the drug layer and the semipermeable membrane.

In another embodiment, the methods comprise orally administering a sustained release dosage form suitable for twice daily oral administration comprising a drug composition containing a high load of a relatively insoluble nonopioid analgesic and a smaller amount of a relatively soluble opioid analgesic, an expandable composition that expands on imbibing water present in the environment of use, and a rate controlling membrane moderating the rate at which the expandable composition imbibes water, wherein said sustained release dosage form provides for proportional release of said nonopioid analgesic and said opioid analgesic over an extended period of time. Preferably the amount of the nonopioid analgesic released from the dosage form (the cumulative release as a percent of the total in the dosage form) is within about 20% of the amount of the opioid analgesic released. In additional embodiments, the amount of the nonopioid analgesic released from the dosage form is within about 10% of the amount of the opioid analgesic released, or within about 5% amount of the opioid analgesic released from the dosage form.

In a preferred embodiment, the methods comprise orally administering to the human patient on a twice-a-day basis an oral sustained release dosage form comprising: (1) a semipermeable wall defining a cavity and including an exit orifice formed or formable therein; (2) a drug layer comprising a therapeutically effective amount of an opioid analgesic and a nonopioid analgesic contained within the cavity and located adjacent to the exit orifice; (3) a push displacement layer contained within the cavity and located distal from the exit orifice; (4) a flow-promoting layer between the inner surface of the semipermeable wall and at least the external surface of the drug layer that is opposite the wall; wherein the dosage form provides an in vitro rate of release of the opioid analgesic and the nonopioid analgesic for up to about 12 hours after being contacted with water in the environment of use.

In an additional embodiment, the invention includes a method for providing an effective amount of an analgesic composition for treating pain in a human patient in need thereof, comprising orally admitting into a patient in need thereof a high load dosage form comprising an effective dose of an opioid analgesic agent and a nonopioid analgesic agent contained in a drug layer and an osmotic push composition, wherein the drug layer and push compositions are surrounded by an at least partially semipermeable wall permeable to the passage of water and impermeable to the passage of said analgesic agents, and an exit means in the wall for delivering the analgesic composition from the dosage form, wherein in operation, water enters through the at least partially semipermeable wall into the dosage form causing the osmotic push composition to expand and push the drug layer through the exit means, wherein the drug layer is exposed to the environment of use as an erodible composition, and wherein the nonopioid analgesic and the opioid analgesic are delivered at a controlled rate over a sustained period of time up to about 12 hours providing a therapeutically effective dose to the patient in need thereof.

In yet additional embodiments, a method for providing an effective concentration of an opioid analgesic and nonopioid analgesic in the plasma of a human patient for the treatment of pain is provided, the method comprising orally admitting into a patient in need thereof a high load dosage form comprising an effective dose of an opioid analgesic agent and a nonopioid analgesic agent contained in a drug layer, an osmotic displacement composition, wherein said drug layer and displacement compositions are surrounded by an at least partially semipermeable wall permeable to the passage of water and impermeable to the passage of said analgesic agents, and an exit means in the wall for delivering the analgesic composition from the dosage form, wherein in operation, water enters through the at least partially semipermeable wall into the dosage form causing the osmotic displacement composition to expand and push the drug layer through the exit means, wherein the drug layer is exposed to the environment of use as an erodible composition, and wherein the nonopioid analgesic and the opioid analgesic are delivered at a proportional rate over a sustained period of time up to about 12 hours.

When administered to a human patient, in certain embodiments, the dosage form produces a plasma profile characterized by a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose. In certain other embodiments, the dosage form produces a minimum Cmax for hydrocodone of about 0.4 ng/mL/mg to a maximum Cmax for hydrocodone of about 1.9 ng/mL/mg and a minimum Cmax for acetaminophen of about 2.0 ng/mL/mg and maximum Cmax for acetaminophen of about 10.4 ng/mL/mg after a single dose. In additional embodiments, the dosage form produces a Cmax for hydrocodone of about 0.8±0.2 ng/mL/mg and a Cmax for acetaminophen of about 4.1±1.1 ng/mL/mg after a single dose.

When administered to the human patient, in certain embodiments, the dosage form produces a Tmax for hydrocodone of about 1.9±2.1 to about 6.7±3.8 hours after a single dose. In other embodiments, the dosage form produces a Tmax for hydrocodone of about 4.3±3.4 hours after a single dose. In certain embodiments, the dosage form produces a Tmax for acetaminophen of about 0.9±0.8 to about 2.8±2.7 hours after a single dose, and in other embodiments, the dosage form produces a Tmax for acetaminophen of about 1.2±1.3 hours after a single dose.

In particular embodiments, when administered to the human patient, the dosage form produces an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose. In additional embodiments, the dosage form produces a minimum AUC for hydrocodone of about 7.0 ng*hr/mL/mg to a maximum AUC for hydrocodone of about 26.2 ng*hr/mL/mg and a minimum AUC for acetaminophen of about 18.4 ng*hr/mL/mg and maximum AUC for acetaminophen of 79.9 ng*hr/mL/mg after a single dose. In yet other embodiments, the dosage form produces an AUC for hydrocodone of about 15.0±3.7 ng*hr/mL/mg and an AUC for acetaminophen of 41.1±12.4 ng*hr/mL/mg after a single dose.

In certain embodiments, the dosage form produces a Cmax for hydrocodone of between about 0.6 ng/mL/mg to about 1.4 ng/mL/mg and a Cmax for acetaminophen of between about 2.8 ng/mL/mg and 7.9 ng/mL/mg, and an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

In yet other embodiments, the dosage form produces a Cmax for hydrocodone of between about 19.6 and 42.8 ng/ml after a single dose of 30 mg hydrocodone, while in other embodiments, the dosage form produces a minimum Cmax for hydrocodone of about 12.7 ng/ml and the maximum Cmax for hydrocodone of about 56.9 ng/mL after a single dose of 30 mg hydrocodone. In a preferred embodiment, the dosage form produces a Cmax for hydrocodone of between about 19.6 and 31 ng/ml after a single dose of 30 mg hydrocodone.

In other embodiments, the dosage form produces a Cmax for acetaminophen of between about 3.0 and about 7.9 μg/ml after a single dose of 1000 mg acetaminophen. In additional embodiments, the dosage form produces a minimum Cmax for acetaminophen of about 2.0 μg/ml and the maximum Cmax of about 10.4 μg/ml after a single dose of 1000 mg acetaminophen. In preferred embodiments, the dosage form produces a Cmax for acetaminophen of between about 3.0 and 5.2 μg/ml after a single dose of 1000 mg acetaminophen.

In other embodiments, the plasma concentration profile for hydrocodone exhibits an area under the concentration time curve between about 275 and about 562 ng*hr/ml after a single dose of 30 mg hydrocodone bitartrate. In additional embodiments, the plasma concentration profile for hydrocodone exhibits a minimum area under the concentration time curve of about 228 ng*hr/ml and a maximum area under the concentration time curve of about 754 ng*hr/ml after a single dose of 30 mg hydrocodone bitartrate.

In particular embodiments, the plasma concentration profile for acetaminophen exhibits an area under the concentration time curve between about 28.7 and about 57.1 ng*hr/ml after a single dose of 1000 mg acetaminophen. In other embodiments, the plasma concentration profile for acetaminophen exhibits a minimum area under the concentration time curve of about 22.5 ng*hr/ml and a maximum area under the concentration time curve of about 72.2 ng*hr/ml after a single dose of 1000 mg acetaminophen.

In yet other embodiments, when administered to the human patient, the dosage form produces a Cmax for hydromorphone of between about 0.12 and about 0.35 ng/ml after a single dose of 30 mg hydrocodone to a non-poor CYP2D6 metabolizer human patient.

In particular embodiments, when administered to the human patient, the plasma concentration for hydrocodone at 12 hours (C12) is between about 11.0 and about 27.4 ng/ml after a single dose of 30 mg hydrocodone bitartrate, and the plasma concentration for acetaminophen at 12 hours (C12) is between about 0.7 and 2.5 μg/ml after a single dose of 1000 mg acetaminophen.

In additional embodiments, the plasma concentration profile exhibits a width at half height value for hydrocodone of between about 6.4 and about 19.6 hours, the plasma concentration profile exhibits a width at half height value for acetaminophen of between about 0.8 and about 12.3 hours.

In particular embodiments, when administered to the human patient, the plasma concentration profile exhibits a weight ratio of acetaminophen to hydrocodone between about 114.2 and 284 at one hour after oral administration of a single dose containing 1000 mg acetaminophen and 30 mg hydrocodone to a human patient. In additional embodiments, the plasma concentration profile exhibits a weight ratio of acetaminophen to hydrocodone between about 70.8 and 165.8 at six hours after oral administration of a single dose containing 1000 mg acetaminophen and 30 mg hydrocodone to a human patient. In yet other embodiments, the plasma concentration profile exhibits a weight ratio of acetaminophen to hydrocodone between about 36.4 and 135.1 at 12 hours after oral administration of a single dose containing 1000 mg acetaminophen and 30 mg hydrocodone to a human patient.

In another aspect, a sustained release dosage form is provided for twice daily oral dosing to a human patient, comprising an immediate release component; and a sustained release component, wherein the immediate release component and the sustained release component collectively provide a therapeutically effective amount of a nonopioid analgesic and an opioid analgesic, wherein the immediate release component and sustained release compoenent provide a means for providing or producing a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose in the plasma of the patient. In additional aspects, the sustained release dosage form provides a means for providing an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 shows a schematic illustration of one embodiment of a dosage form according to the invention.

FIGS. 2A and 2B illustrate the cumulative in vitro release rates of ydrocodone and acetaminophen, respectively, from several representative dosage forms.

FIG. 3 illustrates the cumulative in vitro release rate of acetaminophen and hydrocodone bitartrate from a representative dosage form, showing the proportional release of acetaminophen and hydrocodone from the dosage form.

FIGS. 4A and 4B illustrate the cumulative in vitro release rates of acetaminophen and hydrocodone, respectively, from several representative dosage forms.

FIG. 5A-D illustrate the in vitro release rates and cumulative release of acetaminophen and hydrocodone bitartrate from a representative dosage form having a T₉₀ of about 8 hours.

FIG. 6A-D illustrate the in vitro release rates and cumulative release of acetaminophen and hydrocodone bitartrate from a representative dosage form having a T₉₀ of about 6 hours.

FIG. 7A-D illustrate the in vitro release rates and cumulative release of acetaminophen and hydrocodone bitartrate from a representative dosage form having a T₉₀ of about 10 hours.

FIGS. 8A and B illustrate a comparison between the average in vivo plasma profiles of hydrocodone and acetaminophen, respectively, over a period of 48 hours obtained after a single administration of a representative dosage form and after administration of an immediate release dosage form dosed at zero, four and eight hours.

FIGS. 9A and B illustrate a comparison of the in vivo plasma concentrations of hydrocodone, plotted as concentration or log concentration, respectively, after a single administration of 1, 2 or 3 representative dosage forms and an immediate release dosage form dosed at zero, four and eight hours.

FIGS. 10A and B illustrate a comparison of the in vivo plasma concentrations of acetaminophen, plotted as concentration or log concentration, respectively, after a single administration of a representative dosage form and an immediate release dosage form dosed at zero, four and eight hours.

FIGS. 11A and B illustrate a comparison of the in vivo plasma concentrations of hydromorphone, plotted as concentration or log concentration, respectively, after a single administration of a representative dosage form and an immediate release dosage form dosed at zero, four and eight hours.

FIGS. 12A and B illustrates the mean Cmax and AUC_{∞} (±the standard deviation) observed in patients for the normalized dose of hydrocodone obtained after administering a representative dosage form.

FIGS. 13A and B illustrates the mean Cmax and AUC_{∞} (±the standard deviation) observed in patients for the normalized dose of acetaminophen obtained after administering a representative dosage form.

FIG. 14 illustrates the mean hydrocodone plasma concentration-time profiles at steady state for a representative dosage form dosed every 12 hours and an immediate release dosage form dosed every four hours.

FIG. 15 illustrates the mean hydrocodone trough plasma concentration-time profiles at steady state (±the standard deviation) for a representative dosage form dosed every 12 hours and an immediate release dosage form dosed every four hours.

FIG. 16 illustrates the mean acetaminophen plasma concentration-time profiles at steady state for a representative dosage form dosed every 12 hours and an immediate release dosage form dosed every four hours.

FIG. 17 illustrates the mean acetaminophen trough plasma concentration-time profiles at steady state (±the standard deviation) for a representative dosage form dosed every 12 hours and an immediate release dosage form dosed every four hours.

### DETAILED DESCRIPTION OF THE INVENTION

Definitions and Overview

Before the present invention is described in detail, it is to be understood that unless otherwise indicated this invention is not limited to specific pharmaceutical agents, excipients, polymers, salts, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “a carrier” includes two or more carriers; reference to “a pharmaceutical agent” includes two or more pharmaceutical agents, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For clarity and convenience herein, the convention is utilized of designating the time of drug administration or initiation of dissolution testing as zero hours (t=0 hours) and times following administration in appropriate time units, e.g., t=30 minutes or t=2 hours, etc.

As used herein, the phrase “ascending plasma profile” refers to an increase in the amount of a particular drug in the plasma of a patient over at least two sequential time intervals relative to the amount of the drug present in the plasma of the patient over the immediately preceding time interval. Generally, an ascending plasma profile will increase by at least about 10% over the time intervals exhibiting an ascending profile.

As used herein, the phrase “ascending release rate” refers to a dissolution rate that generally increases over time, such that the drug dissolves in the fluid at the environment of use at a rate that generally increases with time, rather than remaining constant or decreasing, until the dosage form is depleted of about 80% of the drug.

As used herein, the term “AUC” refers to the area under the concentration time curve, calculated using the trapezoidal rule and Clast/k, where Clast is the last observed concentration and k is the calculated elimination rate constant.

As used herein, the term “AUCt” refers to the area under the concentration time curve to last observed concentration calculated using the trapezoidal rule.

As used herein, the term “AUC, ss” refers to the area under the concentration time curve, calculated using the trapezoidal rule, within a 12 hour dosing interval following the sequential administration of the dosage form of the invention every 12 hours for 5 doses.

As used herein, the term “breakthrough pain” refers to pain which the patient experiences despite the fact that the patient is being administered generally effective amounts of an analgesic.

As used herein, the term “Cmax” refers to the plasma concentration of hydrocodone and/or acetaminophen at Tmax expressed as ng/mL and μg/mL, respectively, produced by the oral ingestion of a composition of the invention or the every four hour comparator (NORCO® 10 mg hydrocodone/325 mg acetaminophen). Unless specifically indicated, Cmax refers to the overall maximum observed concentration.

As used herein, the term “Cmax/Cmax, ss” refers to the ratio of the observed maximum concentrations of acetaminophen and hydrocodone following administration of a dosage form of the invention administered sequentially every 12 hours for 5 doses.

As used herein, the term “Cmax/Cmin, ss” refers to the ratio of the observed maximum and minimum acetaminophen and/or hydrocodone concentrations within a 12 hour dosing interval following administration of a dosage form of the invention administered sequentially every 12 hours for 5 doses

As used herein, the term “Cmin/Cmin, ss” refers to the ratio of the observed minimum concentrations of acetaminophen and hydrocodone within a 12 hour dosing interval following administration of a dosage form of the invention administered sequentially every 12 hours for 5 doses.

The term “Cmax, ss” refers to the maximum observed concentration post-administration of a dosage form of the invention administered sequentially every 12 hours for 5 doses

The term “Cmin, ss” refers to the minimum observed concentration within a 12 hour dosing interval of a dosage form of the invention administered sequentially every 12 hours for 5 doses

As used herein, the term “Ctrough, ss” refers to the observed concentration at 12 hours post-administration of a dosage form of the invention administered sequentially every 12 hours for 5 doses.

As used herein, the term “C12” is the plasma concentration of hydrocodone and/or acetaminophen observed at the end of the dosing interval (i.e., about 12 hours) after administration.

The terms “deliver” and “delivery” refer to separation of the pharmaceutical agent from the dosage form, wherein the pharmaceutical agent is able to dissolve in the fluid of the environment of use.

By “dosage form” is meant a pharmaceutical composition or device comprising an active pharmaceutical agent, or a pharmaceutically acceptable acid addition salt thereof, the composition or device optionally containing pharmacologically inactive ingredients, i.e., pharmaceutically acceptable excipients such as polymers, suspending agents, surfactants, disintegrants, dissolution modulating components, binders, diluents, lubricants, stabilizers, antioxidants, osmotic agents, colorants, plasticizers, coatings and the like, that are used to manufacture and deliver active pharmaceutical agents.

As used herein, the term “effective pain management” refers to an objective evaluation of a human patient's response (pain experienced versus side effects) to analgesic treatment by a physician as well as subjective evaluation of therapeutic treatment by the patient undergoing such treatment.

As used herein, the term “fluctuation” refers to the variation in plasma concentrations of hydrocodone and/or acetaminophen computed as 100*(Cmax−Cmin)/Cavg, where Cmin and Cmax were obtained within a 12 hour dosing interval and Cavg is computed as AUC,ss divided by 12, and the term “percent fluctuation” refers to (Cmax−Cmin)/Cmin×100 (for an individual patient). The percent fluctuation for a patient population is defined as (mean Cmax−mean Cmin)/mean Cmin×100.

As used herein, the term “immediate-release” refers to the substantially complete release of drug within a short time period following administration or initiation of dissolution testing, i.e., generally within a few minutes to about 1 hour.

As used herein, the phrase “in vivolin vitro correlation” refers to the correspondence between release of drug from a dosage form as demonstrated by assays measuring the in vitro rate of release of drug from a dosage form and the delivery of drug from a dosage form to a human patient in vivo as demonstrated by assays of drug present in the plasma of the human patient.

As used herein, the term “minimum effective analgesic concentration” refers to the minimum effective therapeutic plasma level of the drug at which at least some pain relief is achieved in a given patient. It will be well understood by those skilled in the medical art that pain measurement is highly subjective and great individual variations may occur among patients.

As used herein, unless further specified, the term “a patient” means an individual patient and/or a population of patients in need of treatment for a disease or disorder.

As used herein, the term “peak width, 50” refers to the time over which 50% of maximum observed concentration is maintained, extrapolating the concentration between observed data points.

By “pharmaceutically acceptable acid addition salt” or “pharmaceutically acceptable salt,” which are used interchangeably herein, are meant those salts in which the anion does not contribute significantly to the toxicity or pharmacological activity of the salt, and, as such, they are the pharmacological equivalent of the base form of the active agent. Examples of pharmaceutically acceptable acids that are useful for the purposes of salt formation include, but are not limited to, hydrochloric, hydrobromic, hydroiodic, sulfuric, citric, tartaric, methanesulfonic, fumaric, malic, maleic and mandelic acids. Pharmaceutically acceptable salts further include mucate, N-oxide, sulfate, acetate, phosphate dibasic, phosphate monobasic, acetate trihydrate, bi(heptafluorobutyrate), bi(methylcarbamate), bi(pentafluoropropionate), bi(pyridine-3-carboxylate), bi(trifluoroacetate), bitartrate, chlorhydrate, and sulfate pentahydrate, benzenesulfonate, benzoate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methyinitrate, methylsulfate, mucate, napsylate, nitrate, pamoate (embonate), pantothenate, phosphate/diphosphate, polygalacturonate, salicylate, stearate, subacetate, succinate, sulfate, tannate, tartrate, teoclate, triethiodide, benzathine, chloroprocaine, choline, diethanolamine, ethylenediamine, meglumine, and procaine, aluminum, calcium, lithium, magnesium, potassium, sodium propionate, zinc, and the like.

As used herein, the term “proportional” (when referring to the release rate or delivery of the nonopioid analgesic and opioid analgesic from the dosage form) refers to the release or the rate of release of the two analgesic agents relative to each other, wherein the amount released is normalized to the total amount of each analgesic in the dosage form, i.e., the amount released is expressed as a percent of the total amount of each analgesic present in the dosage form. Generally, a proportional release rate of the nonopioid analgesic or of the opioid analgesic from the dosage form means that the relative release rate (expressed as percent release) or amount released (expressed as the cumulative release as a percent of the total amount present in the dosage form) of each drug is within about 20%, more preferably within about 10%, and most preferably within about 5% of the release rate or amount released of the other drug. In other words, at any point in time, the release rate of one agent (stated as a percentage of its total mount present in the dosage form) does not deviate more than about 20%, more preferably not more than about 10%, and most preferably not more than about 5% of the release rate of the other agent at the same point in time.

As used herein, the term “ratio, ss” refers to the ratio of plasma concentrations produced by a dosage form of the invention administered every 12 hours for 5 doses relative to an immediate release formulation containing 5 mg hydrocodone and 375 mg acetaminophen administered every 4 hours within a 12 hour dosing.

A drug “release rate” refers to the quantity of drug released from a dosage form per unit time, e.g., milligrams of drug released per hour (mg/hr). Drug release rates for drug dosage forms are typically measured as an in vitro rate of dissolution, i.e., a quantity of drug released from the dosage form per unit time measured under appropriate conditions and in a suitable fluid. For example, dissolution tests can be performed on dosage forms placed in metal coil sample holders attached to a USP Type VII bath indexer and immersed in about 50 ml of acidified water (pH=3) equilibrated in a constant temperature water bath at 37° C. Aliquots of the release rate solutions are tested to determine the amount of drug released from the dosage form, for example, the drug can be assayed or injected into a chromatographic system to quantify the amounts of drug released during the testing intervals.

Unless otherwise specified, a drug release rate obtained at a specified time following administration refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 90% of drug within the dosage form has been released. This measurement is referred to as the “T₉₀” for the dosage form.

As used herein, the term “rescue” refers to a dose of an analgesic which is administered to a patient experiencing breakthrough pain.

Unless specifically designated as “single dose” or at “steady-state,” the pharmacokinetic parameters disclosed and claimed herein encompass both single dose and steady-state conditions.

As used herein, the term “single dose relative” refers to the ratio of plasma concentrations produced by the dosage forms of the invention relative to 10 mg hydrocodone and 325 mg acetaminophen given every 4 hours for a total of 3 doses.

As used herein, the term “sustained release” refers to the release of the drug from the dosage form over a period of many hours. Generally the sustained release occurs at such a rate that blood (e.g., plasma) concentrations in the patient administered the dosage form are maintained within the therapeutic range, that is, above the minimum effective analgesic concentration or “MEAC” but below toxic levels, over a period of time of about 12 hours.

As used herein, the term “Tmax” refers to the time which elapses after administration of the dosage form at which the plasma concentration of hydrocodone and/or acetaminophen attains the maximum plasma concentrations.

As used herein, the phrase “zero order plasma profile” refers to a substantially flat or unchanging amount of a particular drug in the plasma of a patient over a particular time interval. Generally, a zero order plasma profile will vary by no more than about 30% and preferably by no more than about 10% from one time interval to the subsequent time interval.

As used herein, the phrase “zero order release rate” refers to a substantially constant release rate, such that the drug dissolves in the fluid at the environment of use at a substantially constant rate. A zero order release rate can vary by as much as about 30% and preferably by no more than about 10% from the average release rate.

One skilled in the art will understand that effective analgesia will vary according to many factors, including individual patient variability, health status such as renal and hepatic sufficiency, physical activity, and nature and relative intensity of pain.

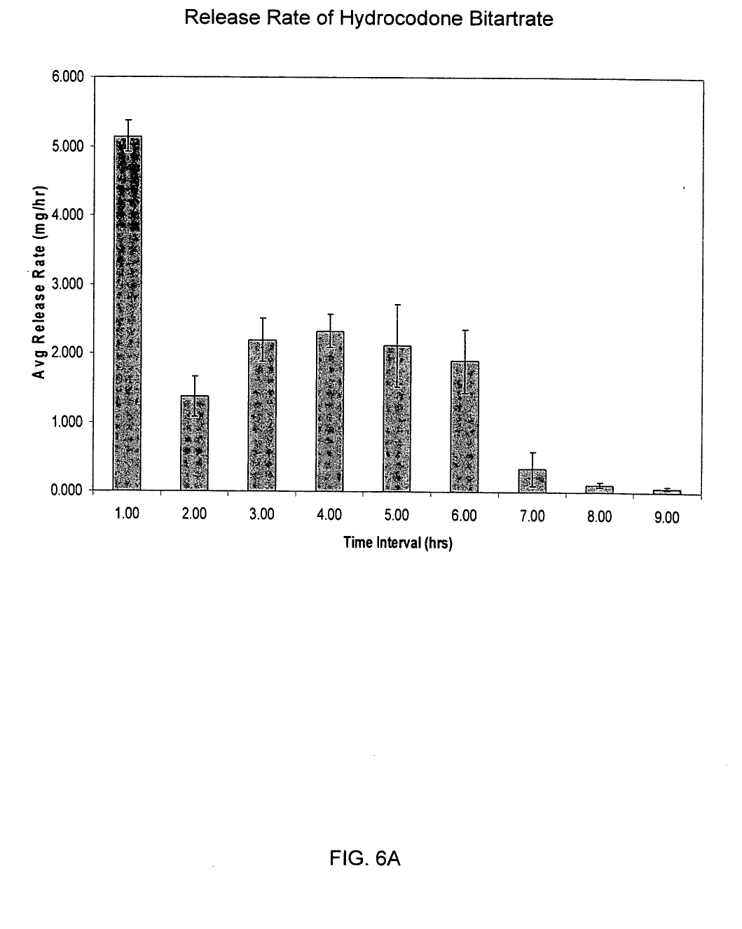

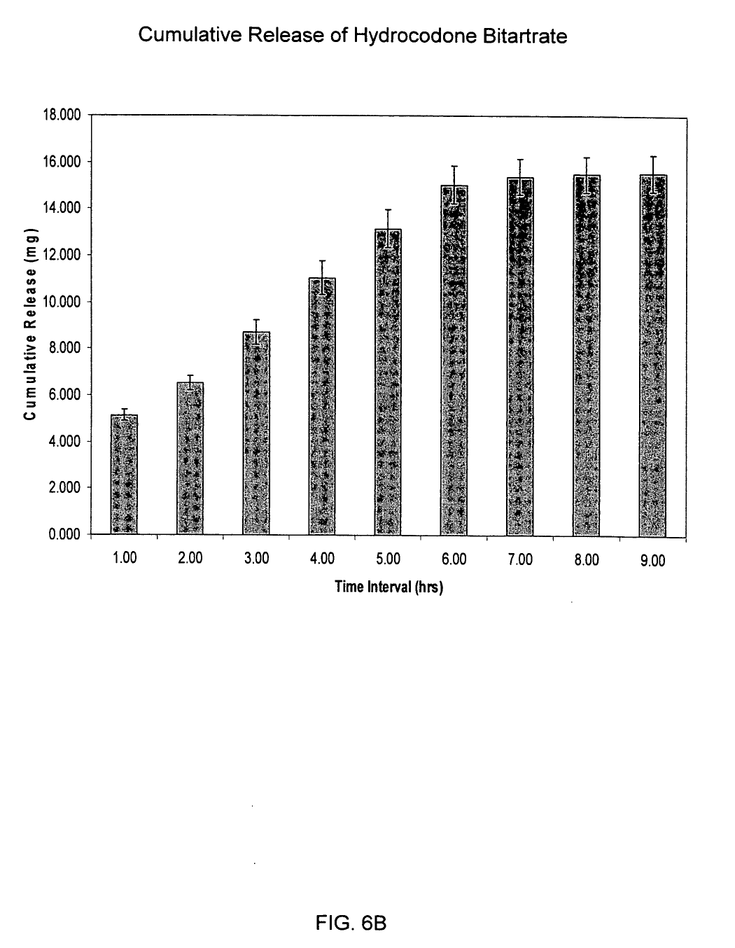

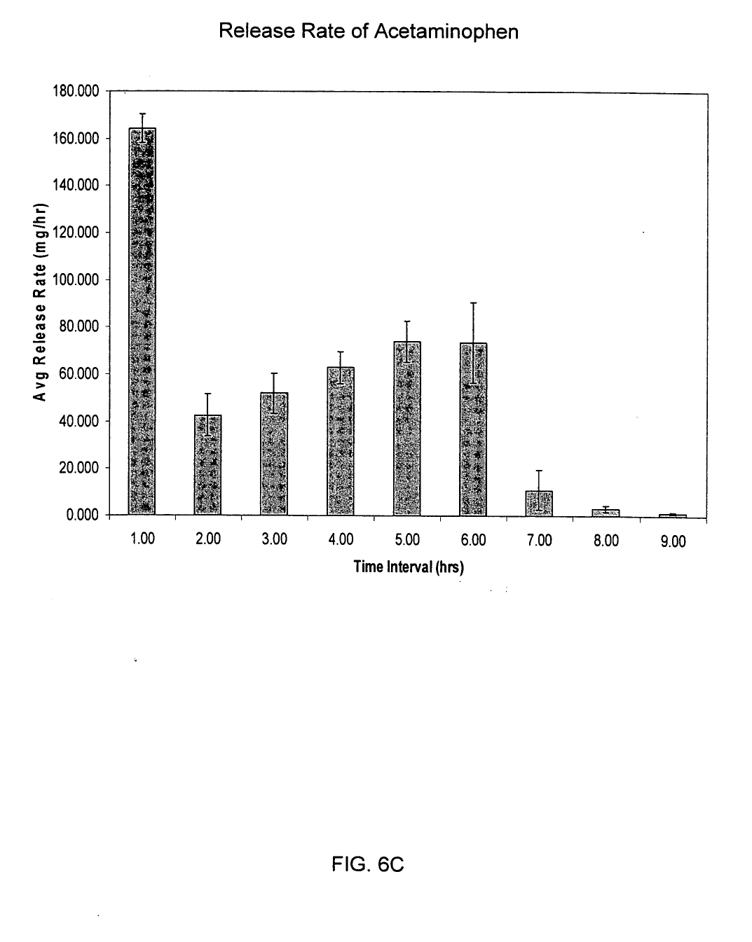

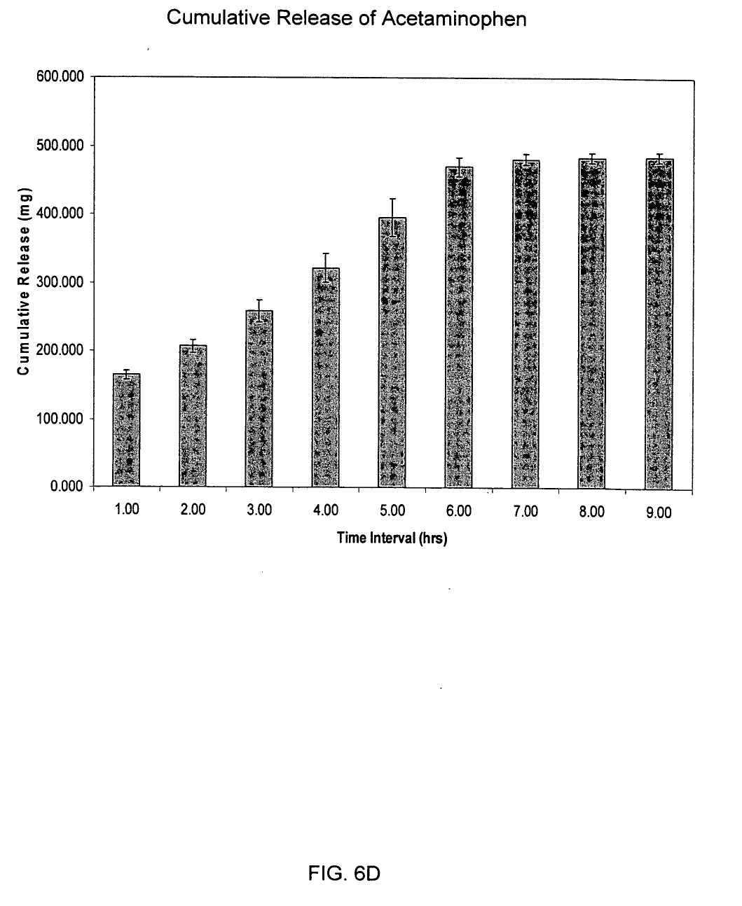

It has been surprisingly discovered that the opioid analgesic and nonopioid analgesic sustained release dosage forms of the present invention provide novel advantages that have not been achieved previously. The presently disclosed formulations provide a high loading of the nonopioid analgesic and exhibit proportional delivery of both the opioid analgesic (e.g., hydrocodone) and nonopioid analgesic (e.g., acetaminophen) in terms of their respective weights in the dosage form, even though the physical properties of the drugs (e.g., their solubilities), differ markedly from each other. The release profile shows a close parallel between the amount of active agent in the drug coating and the sustained release portion of the dosage form and their release profiles from the dosage form, in that the amount released within one hour closely parallels the amount intended to be released immediately into the environment of use, while the amount released in a sustained release profile parallels the amount intended to be released over a prolonged period of time. For example, FIG. 6A shows the dissolution profile of a preferred embodiment, and shows that hydrocodone bitartrate is released at a rate of approximately 5 mg/hr during the first hour of dissolution testing, which closely parallels the amount incorporated into the immediate release drug coating and intended to be released within the first hour of administration. FIG. 6C shows that acetaminophen is released at a rate of approximately 163 mg/hr during the first hour of dissolution testing, which closely parallels the amount incorporated into the immediate release drug coating and intended to be released within the first hour of administration. FIGS. 6B and D show that essentially complete release of the active agent occurred over the period of dissolution testing.

The formulations also show proportional release of the nonopioid analgesic and opioid analgesic relative to one another. For example, as shown in Tables 8 and 9 in Example 4 below, the cumulative acetaminophen release from the 8 hour formulation is 42%, 57% and 89% at 2, 4 and 7 hours post-dissolution testing, respectively. The cumulative hydrocodone bitartrate release from the same formulation is 42%, 61% and 95% at the same time points. Therefore, this formulation exhibits a proportional release of acetaminophen and hydrocodone which are within 0%, 4% and 6% of each other. However, formulations exhibiting nonproportional release characteristics fall within the scope of this invention and the appended claims to the extent that they provide a similar pharmacokinetic profile as that demonstrated herein, especially with regard to the Cmax and AUC values disclosed for hydrocodone bitartrate and acetaminophen.

The formulations can be administered to a human patient in a manner to provide effective concentrations of analgesic to quickly combat existing pain and provide a sustained release to maintain levels of analgesic agents sufficient to alleviate pain or minimize the possibility of breakthrough pain for up to about 12 hours. The dosage forms can be administered to maintain waking hours free of pain as well as before bed time to provide pain free sleep.

Sustained release dosage forms for twice daily oral dosing to a human patient for providing relief from pain are provided. The sustained release dosage form comprises an immediate release component and a sustained release component, wherein the immediate release component and the sustained release component collectively contain a therapeutically effective amount of an opioid analgesic and a therapeutically effective amount of nonopioid analgesic. Preferably, the amount of nonopioid analgesic is between about 20 and about 100 times the amount of opioid analgesic by weight, and in other embodiments, the amount of nonopioid analgesic is between about 20 and about 40 times the amount of opioid analgesic, and in yet other embodiments, the amount of nonopioid analgesic is between about 27 and about 34 times the amount of opioid analgesic by weight.

The sustained release component provides sustained release of each of the opioid analgesic and the nonopioid analgesic at rates proportional to each other. In addition, the immediate release component and the sustained release component provide for proportional release in a quantitative manner. Hence, the amount of each drug present in the immediate release component is delivered to the patient in need thereof substantially immediately (e.g., within one hour), and the amount of each drug present in the sustained release component is released at rates proportional to each other. Further, at least 90% and more preferably at least 95% of each drug contained within the dosage forms is released within the 12 hour dosing period. In preferred embodiments, the dosage forms provide T₉₀'s for both the nonopioid analgesic and the opioid analgesic of between about 6 and about 10 hours, and most preferably, the dosage form provides a T₉₀ of about 8 hours.

In a preferred embodiment, the nonopioid analgesic is acetaminophen and the opioid analgesic is hydrocodone and pharmaceutically acceptable salts thereof, and in preferred embodiments, the pharmaceutically acceptable salt is bitartrate. In certain embodiments, the dosage form contains a loading of acetaminophen of at least 60% by weight, and more typically of between about 75% and about 95% by weight.

In another preferred embodiment, the sustained release dosage form comprises an immediate release component and a sustained release component which collectively contain a therapeutically effective amount of acetaminophen and a therapeutically effective amount of hydrocodone and pharmaceutically acceptable salts thereof, and produces a plasma profile in the patient characterized by a Cmax for hydrocodone of between about 0.6 ng/mL/mg to about 1.4 ng/mL/mg (per mg hydrocodone bitartrate administered) and a Cmax for acetaminophen of between about 2.8 ng/mL/mg and 7.9 ng/mL/mg (per mg acetaminophen administered) and an AUC for hydrocodone of between about 9.1 ng*hr/mL/mg to about 19.9 ng*hr/mL/mg (per mg hydrocodone bitartrate administered) and an AUC for acetaminophen of between about 28.6 ng*hr/mL/mg and about 59.1 ng*hr/mL/mg (per mg acetaminophen administered) after a single dose. In preferred embodiments, the acetaminophen and hydrocodone are present in a weight ratio of between about 20 and about 100, more typically between about 20 and 40, or more preferably, between about 27 and about 34, respectively.

In a preferred embodiment, the dosage form contains about 500±50 mg acetaminophen and 15±5 mg hydrocodone bitartrate, and when a patient is administered a dose of two dosage forms, the dosage form produces a Cmax for hydrocodone of between about 19.4 and 42.8 ng/ml and an area under the concentration time curve between about 275 and about 562 ng*hr/ml after a single dose of 30 mg hydrocodone bitartrate, and a Cmax for acetaminophen of between about 3.0 and about 7.9 μg/ml and an area under the concentration time curve between about 28.7 and about 57.1 μg*hr/ml after a single dose of 1000 mg acetaminophen.

In another embodiment, the sustained release dosage form comprises an analgesic composition comprising a therapeutically effective amount of a nonopioid analgesic and an opioid analgesic; a means for providing an initial release of the nonopioid analgesic and opioid analgesic sufficient to provide an initial peak concentration in the plasma of the human patient, and a means for providing a second release sustained for up to about 12 hours to provide sustained plasma concentrations of the nonopioid analgesic and opioid analgesic sufficient to provide sustained relief from pain for about 12 hours. The means further provides for proportional release of the nonopioid analgesic and opioid analgesic, and at least 90% and more preferably at least 95% of each drug contained within the dosage forms is released within the 12 hour dosing period. In preferred embodiments, the dosage forms provide T₉₀'s for both the nonopioid analgesic and the opioid analgesic of between about 6 and about 10 hours, and most preferably, the dosage form provides a T₉₀ of about 8 hours.

In another embodiment, a controlled release dosage form is provided which is suitable for twice daily oral administration to a human patient for effective relief from pain, comprising: an analgesic composition comprising a therapeutically effective amount of a nonopioid analgesic and an opioid analgesic in a relative weight ratio between about 20 and about 100, more typically between about 20 and 40, and in other embodiments, between about 27 and about 34; and a mechanism providing controlled release of the nonopioid analgesic and opioid analgesic. In preferred embodiments, the release rates of the nonopioid analgesic and opioid analgesic are proportional to each other. In another aspect, the analgesic composition comprises a relatively insoluble nonopioid analgesic at a high drug loading.

In yet another embodiment, a bilayer dosage form of an opioid analgesic and a nonopioid analgesic is provided for twice daily oral administration to a human patient, comprising a drug layer comprising a therapeutically effective amount of the opioid analgesic and nonopioid analgesic, a nondrug layer comprising a high molecular weight polymer providing sustained release of the opioid analgesic and the nonopioid analgesic as an erodible composition upon imbibition of water, a semipermeable membrane providing a controlled rate of entry of water into the dosage form, and a flow promoting layer located between the drug layer and the semipermeable membrane.

In another embodiment, a sustained release dosage form is provided for twice daily oral administration comprising a drug composition containing a high load of a relatively insoluble nonopioid analgesic and a smaller amount of a relatively soluble opioid analgesic, an expandable composition that expands on imbibing water present in the environment of use, and a rate controlling membrane moderating the rate at which the expandable composition imbibes water, wherein said sustained release dosage form provides for proportional release of said nonopioid analgesic and said opioid analgesic over an extended period of time. The high load of a relatively insoluble nonopioid analgesic is at least 60% by weight and more typically between about 75% and about 95% by weight. Preferably the dosage form is suitable for twice daily dosing, and at least 90% and more preferably at least 95% of each analgesic contained within the dosage forms is released within the 12 hour dosing period. In preferred embodiments, the dosage forms provide T₉₀'s for both the nonopioid analgesic and the opioid analgesic of between about 6 and about 10 hours, and most preferably, the dosage form provides a T₉₀ of about 8 hours.

In a preferred embodiment, the sustained release component of the dosage form comprises: (1) a semipermeable wall defining a cavity and including an exit orifice formed or formable therein; (2) a drug layer comprising a therapeutically effective amount of an opioid analgesic and a nonopioid analgesic contained within the cavity and located adjacent to the exit orifice; (3) a push displacement layer contained within the cavity and located distal from the exit orifice; (4) a flow-promoting layer between the inner surface of the semipermeable wall and at least the external surface of the drug layer that is opposite the wall; and the dosage form provides an in vitro rate of release of the opioid analgesic and the nonopioid analgesic for up to about 12 hours after being contacted with water in the environment of use.

Preferably, the drug layer contains a loading of the nonopioid analgesic of at least 60% by weight, and in certain embodiments, the drug layer contains a loading of the nonopioid analgesic of between about 75% and about 95% by weight, and in other embodiments, the drug layer contains a loading of the nonopioid analgesic between about 80% and about 85% by weight. Preferably the drug layer contains a loading of the opioid analgesic between about 1% and about 10% by weight, and in certain embodiments, the drug layer contains a loading of the opioid analgesic between about 2% and about 6% by weight.

The weight ratio of nonopioid analgesic to opioid analgesic can be selected to achieve a desired amount of nonopioid analgesic and opioid analgesic in the dosage form, and generally, the weight ratio of nonopioid analgesic to opioid analgesic can be from about 20 to about 100. The amount of the nonopioid analgesic is more generally between about 20 and about 40 times the amount of the opioid analgesic by weight, or more typically, the amount of the nonopioid analgesic is between about 27 and about 34 times the amount of the opioid analgesic by weight. The weight ratio can also be in the higher range however, and for a dosage form containing 7.5 mg of an opioid analgesic and 500 mg of a nonopioid analgesic, for example, the ratio would be about 67.

Preferably, the dosage form releases the opioid analgesic and the nonopioid analgesic at rates proportional to each other, and the drug layer is exposed to the environment of use as an erodible composition. The in vitro rate of release of the opioid analgesic and the nonopioid analgesic is zero order or ascending. In certain embodiments, the in vitro rate of release of the opioid analgesic and nonopioid analgesic is maintained for from about 6 hours to about 10 hours, and in a preferred embodiment, the in vitro rate of release of the opioid analgesic and nonopioid analgesic is maintained for about 8 hours. In another aspect, at least 90% and more preferably at least 95% of each drug contained within the dosage forms is released within the 12 hour dosing period. In preferred aspects, the dosage forms provide T₉₀'s for both the nonopioid analgesic and the opioid analgesic of between about 6 and about 10 hours, and most preferably, the dosage form provides a T₉₀ of about 8 hours.

In additional embodiments, the dosage form further comprises an immediate release component which preferably comprises a drug coating comprising a therapeutically effective amount of an opioid analgesic and nonopioid analgesic sufficient to provide an analgesic effect in a patient in need thereof. The drug coating provides an immediate release component to the dosage form providing the relatively immediate release and delivery of analgesic agents to the patient in need thereof.

In certain preferred embodiments, the dosage form comprises a therapeutically effective amount of the dose of opioid analgesic and nonopioid analgesic in the drug coating, and the amount in the drug coating is available for immediate delivery to the patient. In such embodiments, the sustained release dosage form exhibits a release rate in vitro of the opioid analgesic and nonopioid analgesic of from about 19% to about 49% released after 0.75 hour, from about 40% to about 70% released after 3 hours, and at least about 80% released after 6 hours. In additional embodiments, the dosage form exhibits a release rate in vitro of the opioid analgesic and nonopioid analgesic of from about 19% to about 49% released after 0.75 hour, from about 35% to about 65% released after 3 hours, and at least about 80% released after 8 hours. In yet other embodiments, the dosage form exhibits a release rate in vitro of the opioid analgesic and nonopioid analgesic of from about 19% to about 49% released after 0.75 hour, from about 35% to about 65% released after 4 hours, and at least about 80% released after 10 hours.

In certain embodiments, the opioid analgesic is selected from hydrocodone, hydromorphone, oxymorphone, methadone, morphine, codeine, or oxycodone, or pharmaceutically acceptable salts thereof, and the nonopioid analgesic is preferably acetaminophen. In a preferred embodiment, the nonopioid analgesic is acetaminophen and the opioid analgesic is hydrocodone bitartrate.

The embodiments of the dosage forms and methods of using them are described in greater detail below.

Drug Coating for Immediate Release of Therapeutic Agents

Drug coating formulations are described in co-pending commonly owned patent application Ser. No. 60/506,195, filed as Attorney Docket No. ARC 3363 P1 on Sep. 26, 2003, incorporated by reference herein in its entirety.

Briefly, the drug coating can be formed from an aqueous coating formulation and includes an insoluble drug, a soluble drug and a water soluble film-forming agent. In a preferred embodiment, the insoluble drug included in the drug coating is a nonopioid analgesic, with acetaminophen being a particularly preferred insoluble drug. In a preferred embodiment, the soluble drug included in the drug coating is an opioid analgesic, with hydrocodone, oxycodone, hydromorphone, oxymorphone, codeine and methadone being particularly preferred soluble drugs.

In preferred embodiments, the drug coating includes from about 85 wt % to about 97 wt % insoluble drug, with coatings exhibiting an insoluble drug loading of about 90 wt % to about 93 wt % being particularly preferred. The total amount of soluble drug included in the drug coating preferably ranges from about 0.5 wt % to about 15 wt % soluble drug, and drug coatings including about 1 wt % to about 3 wt % soluble drug being most preferred. The total amount of insoluble drug included in a drug coating that incorporates both soluble and insoluble drugs preferably ranges from about 60 wt % to about 96.5 wt %, with drug coatings including about 75 wt % to about 89.5 wt % insoluble drug being more preferred, and drug coatings including about 89 wt % to about 90 wt % insoluble drug being most preferred. The total amount of drugs included in the drug coating ranges from about 85 wt % to about 97 wt %, and in preferred embodiments, the total amount of drug included in a drug coating ranges from 10 about 90 wt % to about 93 wt %.

The film-forming agent included in the drug coating is water soluble and accounts for about 3 wt % to about 15 wt % of the drug coating, with drug coatings having about 7 wt % to about 10 wt % film-forming agent being preferred. The film-forming agent included in a drug coating is water soluble and preferably works to solubilize insoluble drug included in the drug coating. In addition, the film-forming agent included in a drug coating may be chosen such that the film-forming agent forms a solid solution with one or more insoluble drugs included in the drug coating. It is believed that drug loading and film forming characteristics of a drug coating are enhanced by selecting a film-forming agent that forms a solid solution with at least one of the one or more insoluble drugs included in the drug coating. A drug dissolved at the molecular level within the film-forming agent (a solid solution) is also expected to be more readily bioavailable because, as the drug coating breaks down or dissolves, the drug is released into the gastrointestinal tract and presented to the gastrointestinal mucosal tissue as discrete molecules.

In a preferred embodiment, the film-forming agent included in the drug coating is a film-forming polymer or a polymer blend including at least one film-forming polymer. Polymer materials used as the film-forming agent of a drug coating are water soluble. Examples of water soluble polymer materials that may be used as the film-forming polymer of a drug coating include, but are not limited to, hydroxypropylmethyl cellulose (“HPMC”), low molecular weight HPMC, hydroxypropyl cellulose (“HPC”) (e.g., Klucel®), hydroxyethyl cellulose (“HEC”) (e.g., Natrasol®), copovidone (e.g., Kollidon® VA 64), and PVA-PEG graft copolymer (e.g., Kollicoat® IR), and combinations thereof. A polymer blend or mixture may be used as the film forming agent in order to achieve a drug coating having characteristics that may not be achievable using a single film-forming polymer in combination with the drug or drugs to be included in the drug coating. For example, blends of HPMC and copovidone provide a film-forming agent that allows the formation of drug coatings that not only exhibit desirable drug loading characteristics, but also provide coatings that are aesthetically pleasing and exhibit desirable physical properties.

The drug coating can also include a viscosity enhancer. Because the drug coating is an aqueous coating that includes an insoluble drug, the drug coating is typically coated from an aqueous suspension formulation. In order to provide a drug coating with substantially uniform drug distribution from a suspension formulation, however, the suspension formulation should provide a substantially uniform dispersion of the insoluble drug included in the coating. Depending on the relative amounts and nature of the film-forming agent and the drugs included in a drug coating, a viscosity enhancer can be included in a drug coating to facilitate the creation of a coating formulation that exhibits sufficient viscosity to provide a substantially uniform drug dispersion and facilitates the production of a drug coating having a substantially uniform distribution of insoluble drug. A viscosity enhancer included in a drug coating is preferably water-soluble and can be a film-forming agent. Examples of viscosity enhancers that may be used in a drug coating include, but are not limited to, HPC (e.g., Klucel®), HEC (e.g., Natrasol®), Polyox® water soluble resin products, and combinations thereof.

The precise amount of viscosity enhancing material included in the drug coating will vary, depending on the amounts and type of film-forming polymer and drug materials to be used in the drug coating. However, where included in a drug coating, a viscosity enhancer will typically account for 5 wt %, or less, of the drug coating. Preferably, a drug coating includes 2 wt %, or less, viscosity enhancer, and in particularly preferred embodiments, the drug coating includes 1 wt %, or less, viscosity enhancer.

The drug coating can also include a disintegrating agent that increases the rate at which the drug coating disintegrates after administration. Because the drug coating typically includes a large amount of insoluble drug, the drug coating may not break down or disintegrate as rapidly as desired after administration. A disintegrating agent included in a coating is a water swellable material that works to structurally compromise the coating as the disintegrating agent absorbs water and swells. Disintegrating agents that may be used in the drug coating include, but are not limited to modified starches, modified cellulose, and cross-linked polyvinylpyrrolidone materials. Specific examples of disintegrating agents that may be used in the drug coating and are commercially available include Ac-Di-Sol®, Avicel®, and PVP XL-10. Where included in the drug coating, a disintegrating agent typically accounts for up to about 6 wt % of the coating, with coatings incorporating from about 0.5 wt % to about 3 wt % being preferred and coatings incorporating from about 1 wt % to about 3 wt % being particularly preferred.

The drug coating can also include a surfactant to increase the rate at which the drug coating dissolves or erodes after administration. The surfactant serves as a “wetting” agent that allows aqueous liquids to more easily spread across or penetrate the drug coating. Surfactants suitable for use in a drug coating are preferably solid at 25° C. Examples of surfactants that may be used in the drug coating include, but are not limited to, surface active polymers, such as Poloxamer and Pluronic® surfactants. Where a surfactant is included in a drug coating, the surfactant will typically account for up to about 6 wt % of the drug coating, with drug coatings including about 0.5 wt % to about 3 wt % surfactant being preferred, and drug coatings including about 1 wt % to about 3 wt % surfactant being particularly preferred.

In one embodiment of the drug coating, the film-forming agent includes a polymer blend formed of copovidone and HPMC. Where such a polymer blend is used as the film-forming agent of the drug coating, the amounts of copovidone and HPMC can vary, as desired, to achieve a drug coating having desired physical and drug-loading characteristics. However, where the film-agent included in a drug coating is formed of a blend of copovidone and HPMC, the copovidone and HPMC are preferably included at a wt/wt ratio about 0.6:1 to about 0.7:1 copovidone to HPMC, with a wt/wt ratio of 1:1.5 being most preferred. Blends of HPMC and copovidone provide drug coatings that are aesthetically pleasing and are believed to be sufficiently robust to withstand further processing and an extended shelf life. Moreover, it is believed that copovidone can work to solubilize insoluble drug included in a drug coating, providing a drug coating that includes a solid solution of insoluble drug.

In a preferred embodiment, the drug coating includes a blend of HPMC and copovidone as the film-forming agent and a nonopioid analgesic as an insoluble drug, preferably acetaminophen.

In yet another embodiment, the drug coating includes a blend of HPMC and copovidone as the film-forming agent, an insoluble nonopioid analgesic, and a soluble opioid analgesic. In a specific example of such an embodiment, the drug coating includes an opioid analgesic, such as hydrocodone and pharmaceutically acceptable salts thereof. A dosage form that includes the combination of acetaminophen and an opioid analgesic provides a combination of analgesic, anti-inflammatory, anti-pyretic, and antitussive actions.

In even further embodiments, the drug coating includes a blend of HPMC and copovidone as the film-forming agent, an insoluble nonopioid analgesic, a soluble opioid analgesic, and a viscosity enhancing agent or a disintegrating agent. In a specific example of such an embodiment, the drug coating includes between about 1 wt % and about 2 wt % of a viscosity enhancing agent, such as HPC. In another example of such an embodiment, the drug coating includes between about 0.5 wt % and about 3 wt % disintegrating agent, and in yet another example of such an embodiment, the drug coating includes between about 0.5 wt % and about 3 wt % of a surfactant.

The drug coating is not only capable of achieving high drug loading, but where the drug coating includes two or more different drugs, it has been found that the drug coating releases the different drugs in amounts that are directly proportional to the amounts of the drugs included in the drug coating. The proportional release is observed even where drugs exhibiting drastically different solubility characteristics, such as acetaminophen and hydrocodone, are included in the drug coating. In addition a drug coating according to the present invention releases substantially all of the drug included therein. Such performance characteristics facilitate reliable and predictable drug delivery performance, and allow formulation of drug coatings that deliver two or more drugs at a wide range of different ratios.

In another aspect, a coating formulation can be used to provide a drug coating. The coating suspension includes the materials used to form a drug coating which is dissolved or suspended, depending on the material, within one or more solvents or solutions. The one or more solvents included in a coating suspension are not organic solvents, and are preferably aqueous solvents. Aqueous solvents that may be used in a coating suspension include, but are not limited to, purified water, pH adjusted water, acidified water, or aqueous buffer solutions. In a preferred embodiment, the aqueous solvent included in a coating suspension is purified water USP. The coating formulation is preferably an aqueous formulation and avoids the potential problems and disadvantages that can result from the use of organic solvents in formulating coating compositions.

As the drug coating includes at least one insoluble drug, the coating formulation is typically prepared as an aqueous suspension using any suitable process, and in preferred embodiments the coating formulation is formulated to facilitate production of drug coatings through a known coating process, such as, for example, pan coating, fluid bed coating, or any other standard coating processes suitable for providing a drug coating. Though the precise amount of solvent used in a coating suspension may vary depending on, for example, the materials to be included in the finished drug coating, the desired coating performance of the coating suspension and the desired physical characteristics of the finished drug coating, a coating suspension typically includes up to about 30 wt % solids content, with the remainder of the coating suspension consisting of the desired solvent. A preferred embodiment of a coating suspension includes about 80 wt % of a desired aqueous solvent and about 20 wt % solids content. The coating suspension is formulated to exhibit a viscosity that is low enough to facilitate spray coating of drug coating, yet is high enough to maintain a substantially uniform dispersion of the insoluble drug included in the coating suspension during a coating process.

In preparing a coating formulation, the drug loaded into the coating formulation can be provided in micronized form. By reducing the particle size of the drug loaded into a coating formulation, a more cosmetically smooth drug coating may be achieved. In addition, by reducing the particle size of the drug material loaded into a coating formulation, the dissolution rate of the drug when released from the drug coating prepared by the coating formulation may be improved, particularly where the drug is an insoluble drug. In one embodiment of the coating formulation, the coating formulation includes a micronized drug material exhibiting an average particle size of less than 100 microns. In another embodiment, the coating formulation includes a micronized drug material exhibiting an average particle size of less than 50 microns, and in yet another embodiment, the coating formulation includes a micronized drug material exhibiting an average particle size of less than 10 microns. Micronization of the drug material can be readily achieved through processes well known in the art, such as, for example, known bead milling, jet milling or microprecipitation processes, and particle size can be measured using any conventional particle size measuring technique, such as sedimentation field flow fractionation, photon correlation spectroscopy or disk centrifugation.

The solids dissolved or suspended in a coating formulation are loaded into the coating formulation in the same relative amounts as are used in a drug coating. For example, the drug included in a coating formulation accounts for about 85 wt % to about 97 wt % of the solids loaded into the coating formulation. In preferred embodiments, the drug included in a coating formulation accounts for about 90 wt % to about 93 wt % of the solids loaded into the coating formulation. The film-forming agent included in a coating formulation accounts for about 3 wt % to about 15 wt % of the solids loaded into the coating formulation, and in preferred embodiments, the film-forming forming agent included in a coating formulation accounts for about 7 wt % to about 10 wt % of the solids loaded into the coating formulation. Where included, a viscosity enhancer will typically account for 5 wt %, or less, of the solids included in a coating formulation. Coating formulations wherein the viscosity enhancer accounts for 2 wt %, or less, of the solids are preferred, and in particularly preferred embodiments, a viscosity enhancer included in a coating formulation accounts for 1 wt %, or less, of the solids included in the coating formulation. If the coating to be formed by the coating formulation is to include a disintegrating agent, the disintegrating agent typically accounts for up to about 6 wt % of the solids included in the coating formulation. In preferred embodiments, a disintegrating agent will account for about 0.5 wt % to about 3 wt % of the solids included in the coating formulation, and in particularly preferred embodiments of a coating formulation including a disintegrating agent, the disintegrating agent accounts for about 1 wt % to about 3 wt % of the solids included in the coating formulation. Where a surfactant is included in a drug coating according to the present invention, the surfactant will typically account for up to about 6 wt % of the solids included in the coating formulation. Preferably, if a surfactant is included in a coating formulation, the surfactant will account for about 0.5 wt % to about 3 wt % of the solids included in the coating formulation, and in particularly preferred embodiments of a coating formulation that includes a surfactant, the surfactant accounts for about 1 wt % to about 3 wt % of the solids included in the coating formulation.

Preparation of Osmotic Dosage Forms Containing a Nonopioid Analgesic and an Opioid Analgesic

The OROS® technology provides tunable sustained release dosage forms that can provide sustained release of one or more analgesic agents, with or without the use of a drug coating providing immediate release of drug. Various types of osmotic dispensers include elementary osmotic pumps, such as those described in U.S. Pat. No. 3,845,770, mini-osmotic pumps such as those described in U.S. Pat. Nos. 3,995,631, 4,034,756 and 4,111,202, and multi-chamber osmotic systems referred to as push-pull, push-melt and push-stick osmotic pumps, such as those described in U.S. Pat. Nos. 4,320,759, 4,327,725, 4,449,983, 4,765, 989 and 4,940,465, 6,368,626, all of which are incorporated herein by reference. Specific adaptations of OROS® that can be used preferably include the OROS® Push-Stick™ System. A significant advantage to osmotic systems is that operation is substantially pH-independent and thus continues at the osmotically determined rate throughout an extended time period even as the dosage form transits the gastrointestinal tract and encounters differing microenvironments having significantly different pH values. Sustained release can be provided for times as short as a few hours or for as long as the dosage form resides in the gastrointestinal tract.

Osmotic dosage forms utilize osmotic pressure to generate a driving force for imbibing fluid into a compartment formed, at least in part, by a semi-permeable wall that permits diffusion of water but not drug or osmagents, if present. In these osmotic dosage forms, the active agent reservoir(s) is typically formed with an active agent compartment, containing a pharmaceutical agent in the form of a solid, liquid or suspension, as the case may be, and an expandable “push” compartment of a hydrophilic polymer that will imbibe fluid from the stomach, swell and force the active agent out of the dosage form and into the environment of use.

A review of such osmotic dosage forms is found in Santus and Baker (1995), “Osmotic drug delivery: a review of the patent literature,” Journal of Controlled Release 35: 1-21, incorporated in its entirety by reference herein. In particular, the following U.S. Patents, owned by the assignee of the present application, ALZA Corporation, and directed to osmotic dosage forms, are each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; 5,156,850; 5,912,268; 6,375,978; 6,368,626; 6,342,249; 6,333,050; 6,287,295; 6,283,953; 6,270,787; 6,245,357; and 6,132,420.

The core of the dosage form typically comprises a drug layer comprising a dry composition or substantially dry composition formed by compression of the binding agent and the analgesic agents as one layer and the expandable or push layer as the second layer. By “dry composition” or “substantially dry composition” is meant that the composition forming the drug layer of the dosage form is expelled from the dosage form in a plug-like state, the composition being sufficiently dry or so highly viscous that it does not readily flow as a liquid stream from the dosage form under the pressure exerted by the push layer. The drug layer itself has very little osmotic activity relative to the push layer, as the drug, binding agent and disintegrant are not well hydrated, and the drug layer does not flow out of the dosage form as a slurry or suspension. The drug layer is exposed to the environment of use as an erodible composition, in contrast to alternative osmotic dosage forms in which the drug layer is exposed to the environment of use as a slurry or suspension. The drug layer is an erodible composition because it includes very little if any osmagent due to the high drug loading provided as well as the poor solubility of the drug to be delivered.

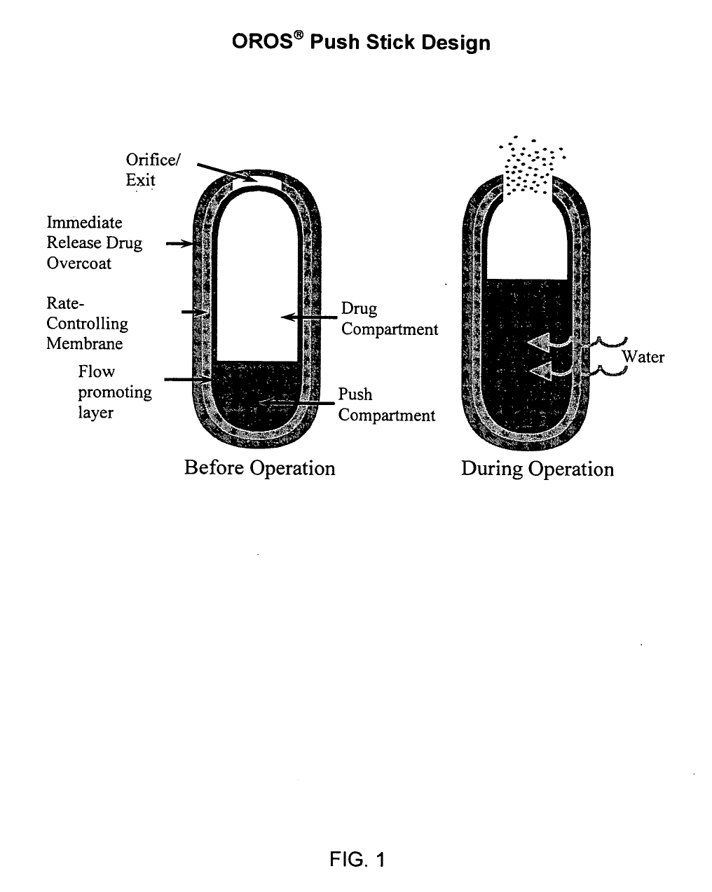

Compression techniques are known in the art and exemplified in Example 1. The expandable layer pushes the drug layer from the exit orifice as the push layer imbibes fluid from the environment of use, and the exposed drug layer will be eroded to release the drug into the environment of use. This may be seen with reference to FIG. 1. Upon release from the dosage form, the drug layer imbibes water causing the disintegrant to swell and soluble agents to dissolve, allowing the erodible solid to disperse and the analgesic agents to dissolve in the fluid at the environment of use. This “push-stick” formulation is a preferred dosage form and is described in greater detail below.

A particular embodiment of the osmotic dosage form comprises: a semipermeable wall defining a cavity and including an exit orifice formed or formable therein, a drug layer comprising a therapeutically effective amount of an opioid analgesic and a nonopioid analgesic contained within the cavity and located adjacent to the exit orifice, a push displacement layer contained within the cavity and located distal from the exit orifice, and a flow-promoting layer between the inner surface of the semipermeable wall and at least the external surface of the drug layer that is opposite the wall. The dosage form provides an in vitro rate of release of the opioid analgesic and the nonopioid analgesic for up to about 12 hours after being contacted with water in the environment of use.

Composition of the Osmotic Dosage Forms

A preferred embodiment of a dosage form of this invention having the “push-stick” configuration is illustrated in FIG. 1 prior to its administration to a subject, during operation and after delivery of the active agent. The dosage form comprises a wall defining a cavity and an exit orifice. Within the cavity and remote from the exit orifice is a push displacement layer, and a drug layer is located within cavity adjacent the exit orifice. A flow-promoting layer extends at least between the drug layer and the inner surface of the wall, and can extend between the inner surface of the wall and the push displacement layer.

The dosage form is at high drug loading, i.e., 60% or greater, but more generally 70% or greater, active agent in the drug layer based on the overall weight of the drug layer, and is exposed to the environment of use as an erodible composition. The drug layer comprises a composition formed of an opioid analgesic, nonopioid analgesic in combination with a disintegrant, a surfactant, a binding agent, and/or a gelling agent, or mixtures thereof. The binding agent is generally a hydrophilic polymer that contributes to the release rate of active agent and controlled delivery pattern, such as a hydroxyalkylcellulose, a hydroxypropylalkylcellulose, a poly(alkylene)oxide, or a polyvinylpyrrolidone, or mixtures thereof. Representative examples of these hydrophilic polymers are poly(alkylene oxides) of 100,000 to 750,000 number-average molecular weight, including without limitation poly(ethylene oxide), poly(methylene oxide), poly(butylene oxide) and poly(hexylene oxide); poly(carboxymethylcelluloses) of 40,000 to 400,000 number-average molecular weight, represented by poly(alkali carboxymethylcellulose), such as poly(sodium carboxymethylcellulose), poly(potassium carboxymethylcellulose) and poly(lithium carboxymethylcellulose); hydroxyalkylcelluloses of 9,200 to 125,000 number-average molecular weight such as hydroxypropylcellulose, hydroxypropylalkylcelluloses such as hydroxypropylalkylcellulose of 9,200 to 125,000 number-average molecular weight, including without limitation, hydroxypropylethylcellulose, hydroxypropyl methylcellulose, hydroxypropylbutylcellulose and hydroxypropylpentylcellulose; and poly(vinylpyrrolidones) of 7,000 to 75,000 number-average molecular weight. Preferred among those polymers are the poly(ethylene oxide) of 100,000-300,000 number average molecular weight and hydroxyalkylcellulose. Carriers that erode in the gastric environment, i.e., bioerodible carriers, are especially preferred.

Surfactants and disintegrants may be utilized in the carrier as well. Disintegrants generally include starches, clays, celluloses, algins and gums and crosslinked starches, celluloses and polymers. Representative disintegrants include corn starch, potato starch, croscarmellose, crospovidone, sodium starch glycolate, Veegum HV, methylcellulose, agar, bentonite, carboxymethylcellulose, low substituted carboxymethylcellulose, alginic acid, guar gum and the like. A preferred disintegrant is croscarmellose sodium.

Exemplary surfactants are those having an HLB value of between about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like. Surfactants that are useful generally include ionic surfactants, including anionic, cationic, and zwitterionic surfactants, and nonionic surfactants. Nonionic surfactants are preferred in certain embodiments and include, for example, polyoxyl stearates such as polyoxyl 40 stearate, polyoxyl 50 stearate, polyoxyl 100 stearate, polyoxyl 12 distearate, polyoxyl 32 distearate, and polyoxyl 150 distearate, and other Myrj™ series of surfactants, or mixtures thereof. Yet another class of surfactant useful in forming the dissolved drug are the triblock co-polymers of ethylene oxide/propylene oxide/ethylene oxide, also known as poloxamers, having the general formula HO(C₂H₄O)a(—C₃H₆O)_{b}(C₂H₄O)ₐH, available under the tradenames Pluronic and Poloxamer. In this class of surfactants, the hydrophilic ethylene oxide ends of the surfactant molecule and the hydrophobic midblock of propylene oxide of the surfactant molecule serve to dissolve and suspend the drug. These surfactants are solid at room temperature. Other useful surfactants include sugar ester surfactants, sorbitan fatty acid esters such as sorbitan monolaurate, sorbitan monopalmitate, sorbitan monostearate, sorbitan tristearate, and other Span™ series surfactants, glycerol fatty acid esters such as glycerol monostearate, polyoxyethylene derivatives such as polyoxyethylene ethers of high molecular weight aliphatic alcohols (e.g., Brij 30, 35, 58, 78 and 99) polyoxyethylene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 2 cetyl ether with butylated hydroxyanisole, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene 40 stearate, polyoxyethylene 50 stearate, polyoxyethylene 100 stearate, polyoxyethylene derivatives of fatty acid esters of sorbitan such as polyoxyethylene 4 sorbitan monostearate, polyoxyethylene 20 sorbitan tristearate, and other Tween™ series of surfactants, phospholipids and phospholipid fatty acid derivatives such as lecithins, fatty amine oxides, fatty acid alkanolamides, propylene glycol monoesters and monoglycerides, such as hydrogenated palm oil monoglyceride, hydrogenated soybean oil monoglyceride, hydrogenated palm stearine monoglyceride, hydrogenated vegetable monoglyceride, hydrogenated cottonseed oil monoglyceride, refined palm oil monoglyceride, partially hydrogenated soybean oil monoglyceride, cotton seed oil monoglyceride sunflower oil monoglyceride, sunflower oil monoglyceride, canola oil monoglyceride, succinylated monoglycerides, acetylated monoglyceride, acetylated hydrogenated vegetable oil monoglyceride, acetylated hydrogenated coconut oil monoglyceride, acetylated hydrogenated soybean oil monoglyceride, glycerol monostearate, monoglycerides with hydrogenated soybean oil, monoglycerides with hydrogenated palm oil, succinylated monoglycerides and monoglycerides, monoglycerides and rapeseed oil, monoglycerides and cottonseed oils, monoglycerides with propylene glycol monoester sodium stearoyl lactylate silicon dioxide, diglycerides, triglycerides, polyoxyethylene steroidal esters, Triton-X series of surfactants produced from octylphenol polymerized with ethylene oxide, where the number “100” in the trade name is indirectly related to the number of ethylene oxide units in the structure, (e.g., Triton X-100™ has an average of N=9.5 ethylene oxide units per molecule, with an average molecular weight of 625) and having lower and higher mole adducts present in lesser amounts in commercial products, as well as compounds having a similar structure to Triton X-100™, including Igepal CA-630™ and Nonidet P-40M (NP-40™, N-lauroylsarcosine, Sigma Chemical Co., St. Louis, Mo.), and the like. Any of the above surfactants can also include optional added preservatives such as butylated hydroxyanisole and citric acid. In addition, any hydrocarbon chains in the surfactant molecules can be saturated or unsaturated, hydrogenated or unhydrogenated.

An especially preferred family of surfactants are the poloxamer surfactants, which are a:b:a triblock co-polymers of ethylene oxide:propylene oxide:ethylene oxide. The “a” and “b” represent the average number of monomer units for each block of the polymer chain. These surfactants are commercially available from BASF Corporation of Mount Olive, N.J., in a variety of different molecular weights and with different values of “a” and “b” blocks. For example, Lutrol® F127 has a molecular weight range of 9,840 to 14,600 and where “a” is approximately 101 and “b” is approximately 56, Lutrol F87 represents a molecular weight of 6,840 to 8,830 where “a” is 64 and “b” is 37, Lutrol F108 represents an average molecular weight of 12,700 to 17,400 where “a” is 141 and “b” is 44, and Lutrol F68 represents an average molecular weight of 7,680 to 9,510 where “a” has a value of about 80 and “b” has a value of about 27.

Other surfactants are the sugar ester surfactants, which are sugar esters of fatty acids. Such sugar ester surfactants include sugar fatty acid monoesters, sugar fatty acid diesters, triesters, tetraesters, or mixtures thereof, although mono- and di-esters are most preferred. Preferably, the sugar fatty acid monoester comprises a fatty acid having from 6 to 24 carbon atoms, which may be linear or branched, or saturated or unsaturated C₆ to C₂₄ fatty acids. The C₆ to C₂₄ fatty acids include C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, and C₂₄, in any subrange or combination. These esters are preferably chosen from stearates, behenates, cocoates, arachidonates, palmitates, myristates, laurates, carprates, oleates, laurates and their mixtures.

Preferably, the sugar fatty acid monoester comprises at least one saccharide unit, such as sucrose, maltose, glucose, fructose, mannose, galactose, arabinose, xylose, lactose, sorbitol, trehalose or methylglucose. Disaccharide esters such as sucrose esters are most preferable, and include sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate.

Particularly preferred examples of these sugar ester surfactants include those sold by the company Croda Inc of Parsippany, N.J. under the names Crodesta F10, F50, F160, and F110 denoting various mono-, di- and mono/di ester mixtures comprising sucrose stearates, manufactured using a method that controls the degree of esterification, such as described in U.S. Pat. No. 3,480,616. These preferred sugar ester surfactants provide the added benefit of tableting ease and nonsmearing granulation.

Use may also be made of those sold by the company Mitsubishi under the name Ryoto Sugar esters, for example under the reference B370 corresponding to sucrose behenate formed of 20% monoester and 80% di-, tri- and polyester. Use may also be made of the sucrose mono- and dipalmitate/stearate sold by the company Goldschmidt under the name “Tegosoft PSE”. Use may also be made of a mixture of these various products. The sugar ester can also be present in admixture with another compound not derived from sugar; and a preferred example includes the mixture of sorbitan stearate and of sucrose cocoate sold under the name “Arlatone 2121” by the company ICI. Other sugar esters include, for example, glucose trioleate, galactose di-, tri-, tetra- or pentaoleate, arabinose di-, tri- or tetralinoleate or xylose di-, tri- or tetralinoleate, or mixtures thereof. Other sugar esters of fatty acids include esters of methylglucose include the distearate of methylglucose and of polyglycerol-3 sold by the company Goldschmidt under the name of Tegocare 450. Glucose or maltose monoesters can also be included, such as methyl O-hexadecanoyl-6-D-glucoside and O-hexadecanoyl-6-D-maltose. Certain other sugar ester surfactants include oxyethylenated esters of fatty acid and of sugar include oxyethylenated derivatives such as PEG-20 methylglucose sesquistearate, sold under the name “Glucamate SSE20”, by the company Amerchol.

A resource of surfactants including solid surfactants and their properties is available in McCutcheon's Detergents and Emulsifiers, International Edition 1979 and McCutcheon's Detergents and Emulsifiers, North American Edition 1979. Other sources of information on properties of solid surfactants include BASF Technical Bulletin Pluronic & Tetronic Surfactants 1999 and General Characteristics of Surfactants from ICI Americas Bulletin 0-1 10/80 5M, and Eastman Food Emulsifiers Bulletin ZM-1K October 1993.

One of the characteristics of surfactants tabulated in these references is the HLB value, or hydrophilic lipophilic balance value. This value represents the relative hydroplicility and relative hydrophobicity of a surfactant molecule. Generally, the higher the HLB value, the greater the hydrophilicity of the surfactant while the lower the HLB value, the greater the hydrophobicity. For the Lutrol® molecules, for example, the ethylene oxide fraction represents the hydrophilic moiety and the propylene oxide fraction represents the hydrophobic fraction. The HLB values of Lutrol F127, F87, F108, and F68 are respectively 22.0, 24.0, 27.0, and 29.0. The preferred sugar ester surfactants provide HLB values in the range of about 3 to about 15. The most preferred sugar ester surfactant, Crodesta F160 is characterized by having a HLB value of 14.5.

Ionic surfactants include cholic acids and derivatives of cholic acid such as deoxycholic acid, ursodeoxycholic acid, taurocholic acid, taurodeoxycholic acid, taurochenodeoxycholic acid, and salts thereof, and anionic surfactants, the most common example of which is sodium dodecyl (or lauryl) sulfate. Zwitterionic or amphoteric surfactants generally include a carboxylate or phosphate group as the anion and an amino or quaternary ammonium moiety as the cation. These include, for example, various polypeptides, proteins, alkyl betaines, and natural phospholipids such as lecithins and cephalins, alkyl-beta-aminopropionates and 2-alkyl-imidazoline quaternary ammonium salts, as well as the CHAPS series of surfactants (e.g., 3-[3-Cholamidopropyl)dimethylammoniol[-1-propanesulfonate hydrate available from Aldrich), and the like.

Surfactants typically have poor cohesive properties and therefore do not compress as hard, durable tablets. Furthermore, surfactants are in the physical form of liquid, pastes, or waxy solids at standard temperatures and conditions and are inappropriate for tableted oral pharmaceutical dosage forms. The aforementioned surfactants have been surprisingly found to function by enhancing the solubility and potential bioavailability of low solubility drugs delivered in high doses.

Surfactant can be included as one surfactant or as a blend of surfactants. The surfactants are selected such that they have values that promote the dissolution and solubility of the drug. A high HLB surfactant can be blended with a surfactant of low HLB to achieve a net HLB value that is between them, if a particular drug requires the intermediate HLB value. The surfactant is selected depending upon the drug being delivered; such that the appropriate HLB grade is utilized.

The nonopioid analgesic can be provided in the drug layer in amounts of from 1 microgram to 1000 mg per dosage form, and more typically from about 200 to about 600 mg, depending upon the required dosing level that must be maintained over the delivery period, i.e., the time between consecutive administrations of the dosage forms, and in a preferred embodiment, the nonopioid analgesic is acetaminophen at 500±50 mg. Generally, loading of compound in the dosage forms will provide doses of the nonopioid analgesic to a subject ranging up to about 3000 mg per day, more usually up to about 1000 to 2000 mg per day, depending on the level of pain being experienced by the patient.

The opioid analgesic can be provided in the drug layer in amounts of from 1 microgram to 50 mg per dosage form, and more typically from about 10 to about 30 mg, depending upon the required dosing level that must be maintained over the delivery period, i.e., the time between consecutive administrations of the dosage forms, and in a preferred embodiment, the opioid analgesic is hydrocodone at 15±5 mg. Generally, loading of compound in the dosage forms will provide doses of the opioid analgesic to a subject ranging up to about 100 mg per day, more between about 10 to 60 mg per day, depending on the level of pain being experienced by the patient.

The push layer is an expandable layer having a push-displacement composition in direct or indirect contacting layered arrangement with the drug layer. The push layer generally comprises a polymer that imbibes an aqueous or biological fluid and swells to push the drug composition through the exit means of the device. Representatives of fluid-imbibing displacement polymers comprise members selected from poly(alkylene oxide) of 1 million to 15 million number-average molecular weight, as represented by poly(ethylene oxide) and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The osmagent, also known as osmotic solute and osmotically effective agent, which exhibits an osmotic pressure gradient across the outer wall and subcoat, comprises a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid raffinose, sucrose, glucose, lactose, sorbitol, inorganic salts, organic salts and carbohydrates.

A flow promoting layer (also called the subcoat for brevity) is in contacting relationship with the inner surface of the semipermeable wall and at least the external surface of the drug layer that is opposite wall; although the flow-promoting layer may, and preferably will, extend to, surround and contact the external surface of the push displacement layer. The wall typically will surround at least that portion of the external surface of the drug layer that is opposite the internal surface of the wall. The flow-promoting layer may be formed as a coating applied over the compressed core comprising the drug layer and the push layer. The outer semipermeable wall surrounds and encases the inner flow-promoting layer. The flow-promoting layer is preferably formed as a subcoat of at least the surface of the drug layer, and optionally the entire external surface of the compacted drug layer and the push displacement layer. When the semipermeable wall is formed as a coat of the composite formed from the drug layer, the push layer and the flow-promoting layer, contact of the semipermeable wall with the flow-promoting layer is assured.

The flow-promoting layer facilitates release of drug from the dosage forms of the invention by reducing the frictional forces between the semipermeable wall 2 and the outer surface of the drug layer, thus allowing for more complete delivery of drug from the device. Particularly in the case of active compounds having a high cost, such an improvement presents substantial economic advantages since it is not necessary to load the drug layer with an excess of drug to insure that the minimal amount of drug required will be delivered.

The flow-promoting layer typically may be 0.01 to 5 mm thick, more typically 0.5 to 5 mm thick, and it comprises a member selected from hydrogels, gelatin, low molecular weight polyethylene oxides (e.g., less than 100,000 MW), hydroxyalkylcelluloses (e.g., hydroxyethylcellulose), hydroxypropylcelluloses, hydroxyisopropylcelluoses, hydroxybutylcelluloses and hydroxyphenylcelluloses, and hydroxyalkyl alkylcelluloses (e.g., hydroxypropyl methylcellulose), and mixtures thereof. The hydroxyalkylcelluloses comprise polymers having a 9,500 to 1,250,000 number-average molecular weight. For example, hydroxypropyl celluloses having number average molecular weights of between 80,000 to 850,000 are useful. The flow promoting layer may be prepared from conventional solutions or suspensions of the aforementioned materials in aqueous solvents or inert organic solvents. Preferred materials for the subcoat or flow promoting layer include hydroxypropyl cellulose, hydroxyethyl cellulose, hydroxypropyl methyl cellulose, povidone [poly(vinylpyrrolidone)], polyethylene glycol, and mixtures thereof. More preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyethyl cellulose and polyethylene glycol prepared in aqueous solution. Most preferably, the flow-promoting layer consists of a mixture of hydroxypropyl cellulose and povidone prepared in ethanol.

Conveniently, the weight of the flow-promoting layer applied to the bilayer core may be correlated with the thickness of the flow-promoting layer and residual drug remaining in a dosage form in a release rate assay such as described herein. During manufacturing operations, the thickness of the flow-promoting layer may be controlled by controlling the weight of the subcoat taken up in the coating operation. When the flow-promoting layer is formed as a subcoat, i.e., by coating onto the tableted bilayer composite drug layer and push layer, the subcoat can fill in surface irregularities formed on the bilayer core by the tableting process. The resulting smooth external surface facilitates slippage between the coated bilayer composite and the semipermeable wall during dispensing of the drug, resulting in a lower amount of residual drug composition remaining in the device at the end of the dosing period. When the flow-promoting layer is fabricated of a gel-forming material, contact with water in the environment of use facilitates formation of a gel or gel-like inner coat having a viscosity that may promote and enhance slippage between the semipermeable wall and the drug layer.

The wall is a semipermeable composition, permeable to the passage of an external fluid, such as water and biological fluids, and substantially impermeable to the passage of active agent, osmagent, osmopolymer and the like. The selectively semipermeable compositions used for forming the wall are essentially nonerodible and are insoluble in biological fluids during the life of the dosage form. The wall need not be semipermeable in its entirety, but at least a portion of the wall is semipermeable to allow fluid to contact or communicate with the push displacement layer such that the push layer can imbibe fluid and expand during use. The wall preferably comprises a polymer such as a cellulose acylate, cellulose diacylate, cellulose triacylate, including without limitation, cellulose acetate, cellulose diacetate, cellulose triacetate, or mixtures thereof. The wall forming material may also be selected from ethylene vinyl acetate copolymers, polyethylene, copolymers of ethylene, polyolefins including ethylene oxide copolymers such as Engage® (DuPont Dow Elastomers), polyamides, cellulosic materials, polyurethanes, polyether blocked amides copolymers such as PEBAX® (Elf Atochem North America, Inc.), cellulose acetate butyrate, and polyvinyl acetate. Typically, the wall comprises 60 weight percent (wt %) to 100 wt % of the cellulosic wall-forming polymer, or the wall can comprise 0.01 wt % to 10 wt % of ethylene oxide-propylene oxide block copolymers, known as poloxamers, or 1 wt % to 35 wt % of a cellulose ether selected from the group consisting of hydroxypropylcellulose and hydroxypropylalkylcellulose and 5 wt % to 15 wt % of polyethylene glycol. The total weight percent of all components comprising the wall is equal to 1 00 wt %.

Representative polymers for forming the wall comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354, Interscience Publishers Inc., New York, N.Y. (1964).

Additional semipermeable polymers for forming the outer wall comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10⁻⁵ to 10⁻² (cc. mil/cm hr. atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff, Eds., CRC Press, Cleveland, Ohio (1971).

The wall may also comprise a flux-regulating agent. The flux regulating agent is a compound added to assist in regulating the fluid permeability or flux through the wall. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as poloxamers (BASF). Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials that may be included in the semipermeable wall material for imparting flexibility and elongation properties to the wall, for making the wall less brittle to nonbrittle and to render tear strength. Suitable materials include phthalate plasticizers such as dibenzyl phthalate, dihexyl phthalate, butyl octyl phthalate, straight chain phthalates of six to eleven carbons, di-isononyl phthalate, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Manufacture of Osmotic Dosage Forms

In brief, the dosage forms are manufactured using the following basic steps, which are discussed in greater detail below. The core, which is a bilayer of one drug layer and one push displacement layer, is formed first and coated with the flow-promoting layer; the coated core can then be dried, though this is optional; and the semipermeable wall is then applied. An orifice is then provided by a suitable procedure (e.g., laser drilling), although alternative procedures can be used which provide an orifice which is formed at a later time (a formable orifice). Finally, the finished dosage forms are dried and are ready for use or for coating with an immediate release drug coating.

The drug layer is formed as a mixture containing the nonopioid analgesic, the opioid analgesic and the binding agent and other ingredients. The drug layer can be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing the drug and binding agent are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Exemplary solvents suitable for manufacturing the respective walls, layers, coatings and subcoatings utilized in the dosage forms of the invention comprise aqueous and inert organic solvents that do not adversely harm the materials utilized to fabricate the dosage forms. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Pan coating may be conveniently used to provide the completed dosage form, except for the exit orifice. In the pan coating system, the subcoat of the wall-forming compositions can be deposited by successive spraying of the respective composition on the bilayered core comprising the drug layer and the push layer accompanied by tumbling in a rotating pan. A pan coater can be used because of its availability at commercial scale. Other techniques can be used for coating the drug core. The coated dosage form can be dried in a forced-air oven, or in a temperature and humidity controlled oven to free the dosage form of solvent. Drying conditions will be conventionally chosen on the basis of available equipment, ambient conditions, solvents, coatings, coating thickness, and the like.

Other coating techniques can also be employed. For example, the semipermeable wall and the subcoat of the dosage form can be formed in one technique using the air-suspension procedure. This procedure consists of suspending and tumbling the bilayer core in a current of air, an inner subcoat composition and an outer semipermeable wall forming composition, until, in either operation, the subcoat and the outer wall coat is applied to the bilayer core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent. An Aeromatic®air-suspension coater can be used employing a cosolvent.

The dosage form of the invention may be manufactured by standard techniques. For example, the dosage form may be manufactured by the wet granulation technique. In the wet granulation technique, the drug and the ingredients comprising the first layer or drug composition are blended using an organic solvent, such as denatured anhydrous ethanol, as the granulation fluid. The ingredients forming the first layer or drug composition are individually passed through a preselected screen and then thoroughly blended in a mixer. Next, other ingredients comprising the first layer can be dissolved in a portion of the granulation fluid, such as the solvent described above. Then, the latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate is added to the drug granulation, then put into milling jars and mixed on ajar mill for 10 minutes. The composition is pressed into a layer, for example, in a Manesty® press. The speed of the press is set at 20 rpm and the maximum load set at 2 tons. The first layer is pressed against the composition forming the second layer and the bilayer tablets are fed to the Kilian® Dry Coater press and surrounded with the drug-free coat, followed by the exterior wall solvent coating.

In another manufacture the nonopioid analgesic and opioid analgesic and other ingredients comprising the first layer facing the exit means are blended and pressed into a solid layer. The layer possesses dimensions that correspond to the internal dimensions of the area the layer is to occupy in the dosage form, and it also possesses dimensions corresponding to the second layer for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, the expandable layer, e.g., a layer of osmopolymer composition, is placed in contact with the layer of drug in a like manner. The layering of the drug formulation and the osmopolymer layer can be fabricated by conventional two-layer press techniques. The two contacted layers are first coated with the flow-promoting subcoat and then an outer semipermeable wall. The air-suspension and air-tumbling procedures comprise in suspending and tumbling the pressed, contacting first and second layers in a current of air containing the delayed-forming composition until the first and second layers are surrounded by the wall composition.

Another manufacturing process that can be used for providing the compartment-forming composition comprises blending the powdered ingredients in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a tote or V-blender. The granules are then pressed in the manner described above.

The flow-promoting layer is then applied to the pressed cores. The semipermeable wall is coated onto the outer surface of the pressed core and/or flow promoting layer. The semi-permeable wall material is dissolved in an appropriate solvent such as acetone or methylene chloride and is then applied to the pressed shape by molding, air spraying, dipping or brushing a solvent-based solution of the wall material onto the shape, as described in U.S. Pat. Nos. 4,892,778 and 4,285,987. Other methods for applying the semi-permeable wall include an air suspension procedure, where the pressed shape is suspended and tumbled in a current of air and wall forming material as described in U.S. Pat. No. 2,799,241, and a pan coating technique.

After application of the semi-permeable wall to the pressed shape, a drying step is generally required and, then, suitable exit means for the active agent must be formed through the semi-permeable membrane. Depending on the properties of the active agent and other ingredients within the cavity and the desired release rate for the dosage form, one or more orifices for active agent delivery are formed through the semi-permeable membrane by mechanical drilling, laser drilling, or the like.

The exit orifice can be provided during the manufacture of the dosage form or during drug delivery by the dosage form in a fluid environment of use. The expression “exit orifice” as used for the purpose of this invention includes a passageway; an aperture; an orifice; or a bore. The orifice may range in size from a single large orifice encompassing substantially an entire surface of the dosage form to one or more small orifices selectively located on the surface of the semi-permeable membrane. The exit orifice can have any shape, such as round, triangular, square, elliptical and the like for the release of a drug from the dosage form. The dosage form can be constructed with one or more exits in spaced apart relation or one or more surfaces of the dosage form.

The exit orifice may be from 10% to 100% of the inner diameter of the compartment formed by the wall, preferably from 30% to 100%, and most preferably from 50% to 100%. In preferred embodiments, the drug layer is released from the dosage form as an erodible solid through a relatively large orifice of a size of at least 100 mils to 100% of the inner diameter of the compartment formed by the wall, typically from about 125 mils (thousandths of an inch) to about 185 mils, or from about 3.175 to about 4.7 mm. The use of a smaller orifice may be employed if desired to provide a further delay in release of the drug layer.

The exit orifice can be performed by drilling, including mechanical and laser drilling, through the outer coat, the inner coat, or both. Exits and equipment for forming exits are disclosed in, for example, U.S. Pat. Nos. 3,845,770 and 3,916,899; in U.S. Pat. No. 4,063,064; and in U.S. Pat. No. 4,088,864.

The exit can also be an orifice that is formed from a substance or polymer that erodes, dissolves or is leached from the outer coat or wall or inner coat to form an exit orifice, as disclosed, for example, in U.S. Pat. Nos. 4,200,098 and 4,285,987. Representative materials suitable for forming an orifice, or a multiplicity of orifices comprise leachable compounds, such as a fluid removable pore-former such as inorganic and organic salts, inorganic or organic oxides, carbohydrates, polymers, such as leachable poly(glycolic) acid or poly(lactic) acid polymers, gelatinous filaments, poly(vinyl alcohol), leachable polysaccharides, sugars such as sorbitol, which can be leached from the wall. For example, an exit, or a plurality of exits, can be formed by leaching sorbitol, lactose, fructose, glucose, mannose, galactose, talose, sodium chloride, potassium chloride, sodium citrate and mannitol from the wall.

In addition, in some embodiments, the osmotic dosage form can be in the form of an extruded tube open at one or both ends, as described in commonly owned U.S. Pat. No. 6,491,683 to Dong, et al. In the extruded tube embodiment, it is not necessary to provide an additional exit means.

Non-Osmotic Sustained Release Dosage Forms

The embodiments of this invention are not limited to a single type of dosage form having a particular mechanism of drug release. This pharmacokinetic profile can in principle be obtained using additional non-osmotic oral sustained release dosage forms, as described in greater detail below.

As of the filing date of this application, there are three types of commonly used oral controlled release dosage forms. They include matrix systems, osmotic pumps, and membrane controlled technologies (also referred to as reservoir systems), summarized in Table 1 below. A detailed discussion of such dosage forms may also be found in Handbook of Pharmaceutical Controlled Release Technology, ed. D. L. Wise, Marcel Dekker, Inc., New York, N.Y. (2000), and Treatise on Controlled Drug Delivery, Fundamentals, Optimization, and Applications, ed. A. Kydonieus, Marcel Dekker, Inc., New York, N.Y. (1992), the contents of each which is hereby incorporated by reference.

TABLE 1
Common Oral Controlled Release Systems
Feasible for Commercial Development

| | Reservoir | |
|---|---|---|
| Matrix Systems | Systems | Osmotic Systems |
| Hydrophilic | Coated beads | Elementary |
| matrix | or tablets | osmotic pump |
| Swellable | | |
| Swellable and | Microencapsulation | Push-Pull ™ |
| erodible | | system |
| Hydrophobic | | Push-Layer ™ |
| matrix | | system |
| Homogenous | | |
| (non-porous) | | |
| Heterogeneous | | Push-Stick ™ |
| (porous) | | system |
| Inert | | |
| (monolithic) | | |
| Erodible | | |
| Degradable | | |

Matrix Systems

Matrix systems are well known in the art. In a matrix system, the drug is homogenously dispersed in a release rate controlling matrix in association with conventional excipients. This admixture is typically compressed under pressure to produce a tablet. Drug is released from this tablet by diffusion and/or erosion. Matrix systems are described in detail by Wise and Kydonieus, supra. In a matrix system, a drug is incorporated into the polymer matrix by either particle or molecular dispersion. The former is simply a suspension of drug particles homogeneously distributed in the matrix, while the latter is a matrix with drug molecules dissolved in the matrix. Drug release occurs either by diffusion and/or erosion of the matrix system.

In a hydrophilic matrix, there are two competing mechanisms involved in the drug release: Fickian diffusional release and relaxational release. Diffusion is not the only pathway by which a drug is released from the matrix; the erosion of the matrix following polymer relaxation also contributes to the overall release. The relative contribution of each component to the total release is primarily dependent upon the properties of a given drug. For instance, the release of a sparingly soluble drug from hydrophilic matrices involves the simultaneous absorption of water and desorption of drug via a swelling-controlled diffusion mechanism. As water penetrates into a glassy polymeric matrix, the polymer swells and its glass transition temperature is lowered. At the same time, the dissolved drug diffuses through this swollen rubbery region into the external releasing medium. This type of diffusion and swelling generally does not follow a Fickian diffusion mechanism.

In a hydrophobic inert matrix system, the drug is dispersed throughout a matrix that involves essentially negligible movement of the device surface. For a homogeneous monolithic matrix system, the release behavior can be described by the Higuchi equation subject to the matrix-boundary conditions. See Higuchi, T. (1961) “Rate of Release of Medicaments from Ointment Bases Containing Drugs in suspension,” J. Pharm. Sci., 50:847.

Drug release from a porous monolithic matrix system involves the simultaneous penetration of surrounding liquid, dissolution of drug, and leaching out of the drug through interstitial channels or pores. The volume and length of the openings in the matrix must be accounted for in a more complex diffusion equation. Thus, in contrast to the homogeneous monolithic matrix system, the release from a porous monolith is expected to be directly proportional to the drug concentration in the matrix

The matrix formulations of this invention comprise an opioid analgesic, nonopioid analgesic and a pharmaceutically acceptable polymer. Preferably, the opioid analgesic is hydrocodone and pharmaceutically acceptable salts thereof. Preferably the nonopioid analgesic is acetaminophen. The amount of the nonopioid analgesic varies from about 60% to about 95% by weight of the dosage form, and the amount of opioid analgesic varies from about 1% to about 10%. Preferably, the dosage form comprises about 75% to about 85% by weight of acetaminophen.

The pharmaceutically acceptable polymer is a water-soluble hydrophilic polymer, or a water insoluble hydrophobic polymer or nonpolymer waxes. Examples of suitable water soluble polymers include polyvinylpyrrolidine, hydroxypropylcellulose, hydroxypropylmethyl cellulose, methyl cellulose, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable waxes include fatty acids and glycerides.

Preferably, the polymer is selected from hydroxypropyl cellulose, hydroxypropylmethyl cellulose, and methyl cellulose. More preferably, the polymer is hydroxypropylmethyl cellulose. Most preferably, the polymer is a high viscosity hydroxypropyl-methyl cellulose with viscosity ranging from about 4,000 cps to about 100,000 cps. The most preferred high viscosity polymer is a hydroxypropylmethyl cellulose with a viscosity of about 15,000 cps, commercially available under the Tradename, Methocel, from The Dow Chemical Company. The amount of the polymer in the dosage form generally varies.

The composition of the invention also typically includes pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, binders, etc. Such excipients are routinely used in the dosage forms of this invention.

Diluents, or fillers, are added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc.

Glidants are also typically incorporated into the formulation. A glidant improves the flow characteristics of the granulation. Examples of suitable glidants include talc, silicon dioxide, and cornstarch.

Binders may be incorporated into the formulation. Binders are typically utilized if the manufacture of the dosage form uses a granulation step. Examples of suitable binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, antioxidants, or any other excipient commonly used in the pharmaceutical industry, etc. The amount of excipients used in the formulation will correspond to that typically used in a matrix system. The total amount of excipients, fillers and extenders, etc. varies.

The matrix formulations are generally prepared using standard techniques well known in the art. For example, they can be prepared by dry blending the polymer, filler, nonopioid analgesic, opioid analgesic, and other excipients followed by granulating the mixture using an appropriate solvent until proper granulation is obtained. The granulation is done by methods known in the art. The wet granules are dried in a fluid bed dryer, sifted and ground to appropriate size. Lubricating agents are mixed with the dried granulation to obtain the final formulation.

The compositions of the invention can be administered orally in the form of tablets, pills, or the granulate may be loose filled into capsules. The tablets can be prepared by techniques known in the art and contain a therapeutically useful amount of the nonopioid analgesic, opioid analgesic and such excipients as are necessary to form the tablet by such techniques. Tablets and pills can additionally be prepared with enteric coatings and other release-controlling coatings for the purpose of acid protection, easing swallow ability, and controlling drug release, etc. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

Reservoir Polymeric Systems

Fick's first law of diffusion may be used to characterize the release rate of a drug from a reservoir polymeric system at steady-state. The apparent zero-order or near-zero-order release from this type of system is often desired for a controlled release dosage form in many situations.

In developing reservoir polymeric systems, commonly used methods include microencapsulation of drug particles, coating of tablets or multiparticulates, and press-coating of tablets. A polymeric membrane or press-coated layer offers a predetermined resistance to drug diffusion from the reservoir to the sink. The driving force of such systems is the concentration gradient of active molecules between reservoir and sink. In the case of film coating, the resistance provided by the membrane is a function of film thickness and characteristic of both the film as well as the migrating species in a given environment. The mechanisms of drug release from the film-coated dosage forms may be categorized into 1) transport of the drug through a network of capillaries filled with dissolution media; 2) transport of the drug through the homogeneous film barrier by diffusion; 3) transport of the drug through a hydrated swollen film; and 4) transport of the drug through flaws, cracks and imperfections within the coating matrix. See, Donbrow, M. and Friedman, M., (1975) “Enhancement of Permeability of Ethyl Cellulose Films for Drug Penetration,” J. Pharm. Pharmacol., 27:633; Donbrow, M. and Samuelov, Y. (1980) “Zero Order Drug Delivery from Double-Layered Porous Films: Release Rate Profiles from Ethyl Cellulose, Hydroxypropyl Cellulose and Polyethylene Glycol Mixtures,” J. Pharm. Pharmacol., 32:463; and Rowe, R. C. (1986) “The Effect of the Molecular Weight of Ethyl Cellulose on the Drug Release Properties of Mixed Films of Ethyl Cellulose and Hydroxypropyl Methylcellulose,” Int. J. Pharm., 29:37-41. Examples of such systems are described in U.S. Pat. No. 6,387,404 to Oshlack.

The reservoir sustained release system of this invention comprises an opioid analgesic, nonopioid analgesic and pharmaceutically acceptable polymer(s). Preferably, the opioid analgesic is hydrocodone and pharmaceutically acceptable salts thereof. Preferably the nonopioid analgesic is acetaminophen. The amount of the nonopioid analgesic varies from about 40% to about 90% by weight of the dosage form, and the amount of opioid analgesic varies from about 1% to about 10%. Preferably, the dosage form comprises about 55% to about 75% by weight of acetaminophen.

The pharmaceutically acceptable polymer include hydrophobic polymer, hydrophilic polymer or nonpolymer release rate-controlling materials. Examples of suitable water hydrophilic polymers include polyvinylpyrrolidine, hydroxypropylcellulose, hydroxypropylmethyl cellulose, methyl cellulose, polyethylene glycol, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable nonpolymer materials include fatty acids and glycerides, long carbon chain fatty acid esters, low molecular weight polyethylene.

Preferably, the release rate controlling polymer is often selected from ethylcellulose (Surelease from Colorcon, Aquacoat ECD from FMC), ammoniomethacrylate copolymers, methacrylic ester copolymers (Eudragit RL, RS, NE30D from Rohm America). The pore former in the membrane is often selected from hydroxypropyl cellulose, hydroxypropylmethyl cellulose, and polyethylene glycol. The amount of the polymer in the dosage form generally varies.

The composition of the invention also typically includes pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, binders, etc. Such excipients are routinely used in the dosage forms of this invention.

Diluents, or fillers, are added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc.

Glidants are also typically incorporated into the formulation. A glidant improves the flow characteristics of the granulation. Examples of suitable glidants include talc, silicon dioxide.

Binders may be incorporated into the formulation. Binders are typically utilized if the manufacture of the dosage form uses a granulation step. Examples of suitable binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, plasticizers, antioxidants, or any other excipient commonly used in the pharmaceutical industry, etc. The amount of excipients used in the formulation will correspond to that typically used in a reservoir system. The total amount of excipients, fillers and extenders, etc. varies.

The reservoir formulations in the form of tablet or beads are generally prepared using techniques well known in the art. For example, tablet core are prepared by dry blending the filler, nonopioid analgesic, opioid analgesic, polymer and other excipients followed by granulating the mixture using an appropriate solvent until proper granulation is obtained. The granulation is done by methods known in the art. The wet granules are dried in a fluid bed dryer, sifted and ground to appropriate size. Lubricating agents are mixed with the dried granulation to obtain the final formulation. The tablet can also be produced by dry granulation or direct compression. Beads used as substrates for coating are often prepared by extrusion/spheronization, use of non-peril seeds or granulation techniques.

Film coating of the tablets or beads with rate controlling polymers are performed using techniques well known in the art, such as pan coating or fluid-bed coating. Other coating techniques include compression coat using tableting machine. For example, to achieve proportional release of the opioid and nonopioid analgesics of this invention, separate coating of opioid and nonopioid analgesics are performed followed by combining them into a single unit dosage form (tablet, capsule), or alternatively, partial coating of tablet core in the form of layered tablet are used. The reservoir system is also prepared by coating a matrix tablet core using film or press coating to provide dual control of drug release from the reservoir system.

The compositions of the invention can be administered orally in the form of tablets, pills, or the granulate may be loose filled into capsules. The tablets can be prepared by techniques known in the art and contain a therapeutically useful amount of the nonopioid analgesic, opioid analgesic and such excipients as are necessary to form the tablet by such techniques. Tablets and pills can additionally be prepared with enteric coatings and other release-modifying coatings for the purpose of acid protection, modified release, easing swallow ability, etc. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

To illustrate additional embodiments that are not limited to a single type of system (i.e. osmotic dosage forms), various matrix or reservoir systems have been designed which are intended to obtain in vivo performance equivalent to the osmotic dosage forms tested in clinical trials. These designs include layered matrix tablets (see Examples 8-12, 20), multi-unit matrix tablets (see Examples 13-14), compression coated matrix tablets (see Example 15), and multi-unit reservoir tablets (see Examples 16-19). These examples also demonstrate that release of acetaminophen and hydrocodone from these additional types of solid dosage forms can be tailored by altering formulation composition and, in some cases, processing conditions etc.

The state of the art is such that similar in vitro drug release from different types of designs may not always translate into equivalent in vivo performance in humans. In addition, drug release from many types of systems is known to vary with test methodology and conditions while the osmotic dosage forms are generally insensitive to such changes. Thus, to obtain equivalent in vivo performance using a different type of system (such as those illustrated, but not limited to, in Examples 8-20), one would test a selected formulation having an in vitro release rate similar to that of the osmotic dosage forms in humans using a cross-over study design, such as those described in Examples 5-7, to determine the in vivo performance of the formulation (e.g., the resulting pharmacokinetic profile, efficacy, etc.). In the absence of information regarding the in vitro/in vivo correlation for various systems, the likely in vivo outcomes of the study would include: (1) the test formulation is equivalent to osmotic dosage forms; (2) the test formulation releases active agents faster than the osmotic dosage forms; (3) the test formulation releases active agents slower than the osmotic dosage forms.

For outcome (2), one would make formulation adjustments in the test formulation to slow down the in vitro release rate in order to achieve in vivo equivalence. These adjustments include, but are not limited to, increasing the proportion of release controlling materials in the formulation (e.g. glyceryl behenate, ethylcellulose etc.) and decreasing the proportion of water soluble excipients (e.g. lactose, HPC, etc.) in the matrix or in the coating film.

For outcome (3), one would make formulation adjustments to speed up the in vitro release rate in order to achieve in vivo equivalence. These adjustments include, but are not limited to, decreasing the proportion of release controlling materials in the formulation (e.g. glyceryl behenate, ethylcellulose etc.) and increasing the proportion of water soluble excipients (e.g. lactose, HPC, etc.) in the matrix or in the coating film.

Therefore, examples 8-20 demonstrate the ability of different types of systems to obtain a range of in vitro drug release rates that are similar, faster or slower than that of the osmotic dosage forms, thus providing more latitude (flexibility) in generating dosage forms that can produce equivalent in vivo performance of the osmotic dosage forms.

Nonopioid Analgesic Agents

A wide variety of nonopioid analgesic agents may be used in combination with a suitable opioid analgesic agent in the dosage form to provide sustained release of analgesic agents to a patient in need thereof on a twice daily basis. In particular, poorly soluble analgesic agents such as acetaminophen can be employed at high loading to provide pain relief for an extended period of time. Examples of nonopioid analgesics include the poorly soluble para-aminophenol derivatives exemplified by acetaminophen, aminobenzoate potassium, aminobenzoate sodium. A preferred nonopioid analgesic agent is acetaminophen. The dose of nonopioid analgesic agents is typically 0.5 mg to 600 mg, and is generally in the range of about 1 mg to about 1000 mg, and more typically between about 300 mg and about 500 mg.

Opioid Analgesic Agents

Opioid analgesics generally include, without limitation: alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacyl morphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, salts thereof and mixtures thereof. Particularly preferred opioid analgesics include hydrocodone, hydromorphone, codeine, methadone, oxymorphone, oxycodone, and morphine.

Methods of Use

The dosage forms described above can be used in a variety of methods. For example, the dosage forms can be used in methods for providing an effective concentration of an opioid analgesic and nonopioid analgesic in the plasma of a human patient for the treatment of pain, methods for treating pain in a human patient, methods for providing sustained release of a nonopioid analgesic and opioid analgesic, and methods for providing an effective amount of an analgesic composition for treating pain in a human patient in need thereof, and so forth.

As described in detail in Examples 5 and 6, clinical trials were performed to determine the bioavailability of the sustained release dosage forms described herein, as well as their bioequivalence to an immediate release dosage form dosed every four hours ((NORCO® 10/325). The pharmacokinetic parameters produced in human patients are presented in Tables 2-4 and discussed further below.

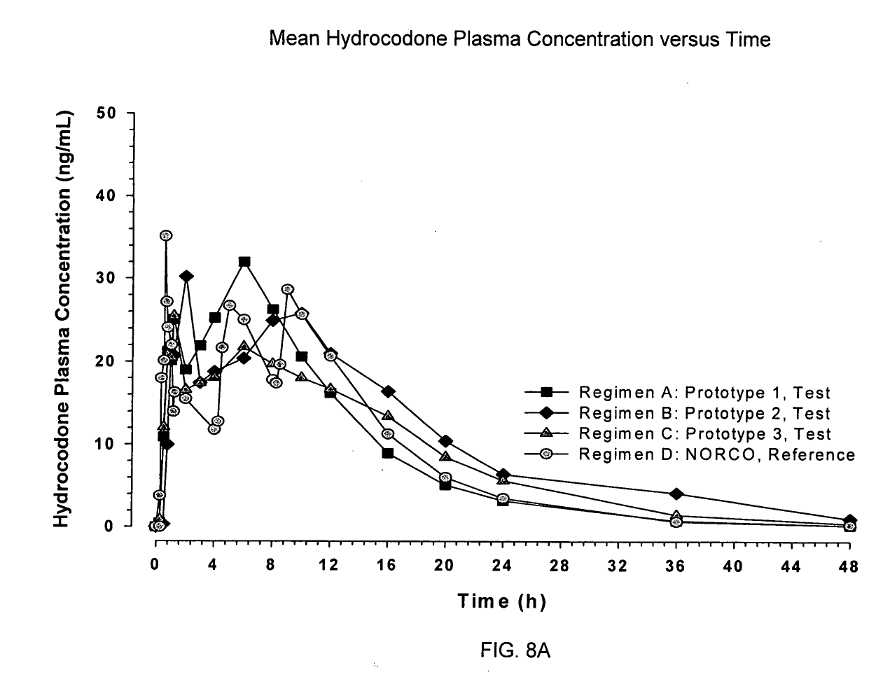

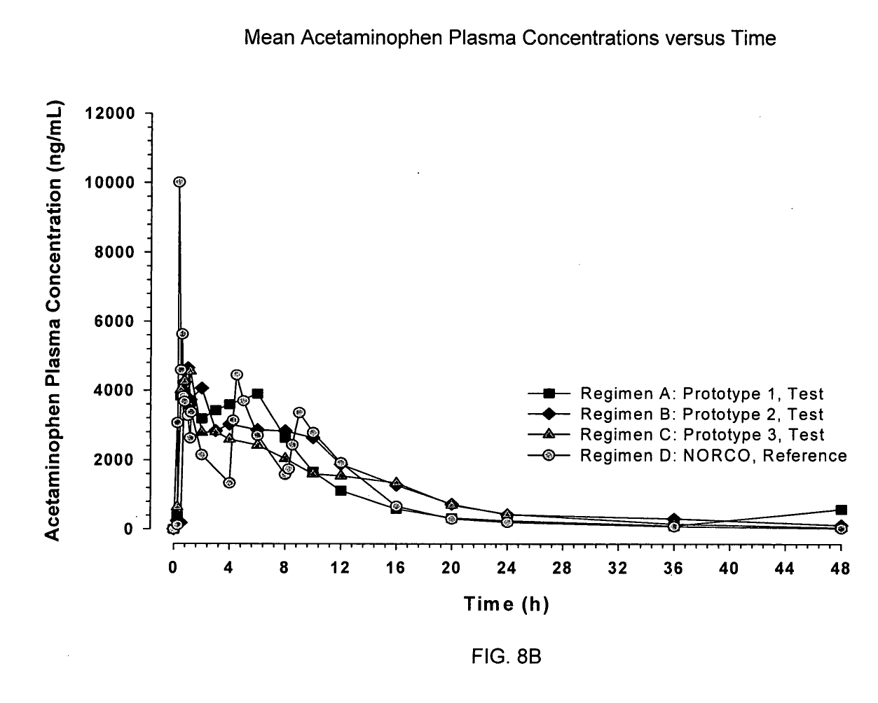

In the first clinical study, bioavailability of several representative dosage forms and their bioequivalence with an immediate release dosage form (NORCO® 10/325, 1 tablet every 4 hours for 3 doses) was demonstrated. Dosage forms having a variety of release rates, producing T₉₀'s of approximately 6, 8 and 10 hours, were tested. Tables 2-4 and FIGS. 8A and B illustrate the comparison between the mean in vivo plasma profiles of hydrocodone and acetaminophen observed after administration of representative dosage forms having T₉₀'s of approximately 6, 8 and 10 hours, and after administration of the immediate release dosage form comprising acetaminophen and hydrocodone bitartrate every four hours. As these figures illustrate, volunteers receiving two tablets of each of the three dosage forms prepared according the procedure of Example 1 exhibited a rapid rise in plasma concentrations of hydrocodone and acetaminophen after oral administration at time zero. The dosage forms produced a rapid rise in plasma levels of hydrocodone and acetaminophen, followed by a sustained release of hydrocodone and acetaminophen sufficient to provide therapeutically effective levels in the plasma of the patients for an extended period of time, suitable for twice daily dosing. Subsequent to the initial release of hydrocodone and acetaminophen, the sustained release of the dosage forms provides for continued release of hydrocodone and acetaminophen to the patient.

All three of the dosage forms in Regimens A, B and C produced an ascending plasma profile of hydrocodone (see FIG. 8A), while only Regimen A produced an ascending plasma profile of acetaminophen. Regimens B and C, with their slower rate of release of drug, provided acetaminophen at a rate that produced a zero order or even descending plasma profile of acetaminophen, due to the rapid metabolism of this drug. Thus depending on the pharmacokinetic properties of the drug and the individual patient's metabolism, an ascending rate of release of drug in vitro can manifest in vivo as an ascending, zero order or descending plasma profile.

The test Regimens A (6 hour release prototype), B (8 hour release prototype) and C (10 hour release prototype) were equivalent to the reference Regimen D (NORCO®) with respect to AUC for both hydrocodone and acetaminophen because the 90% confidence intervals for evaluating bioequivalence were contained within the 0.80 to 1.25 range. Test Regimen A was equivalent to the reference Regimen D with respect to hydrocodone Cₘₐₓ because the 90% confidence interval for evaluating bioequivalence was contained within the 0.80 to 1.25 range. Compared to Regimen D, hydrocodone Cₘₐₓ central values for Regimens B and C were 16% and 25% lower, and acetaminophen Cₘₐₓ central values for Regimens A, B and C were 9% to 13% lower. The decrease in Cₘₐₓ while maintaining AUC levels provided by the sustained release dosage forms provides a dosage form that should be less likely to result in adverse events.

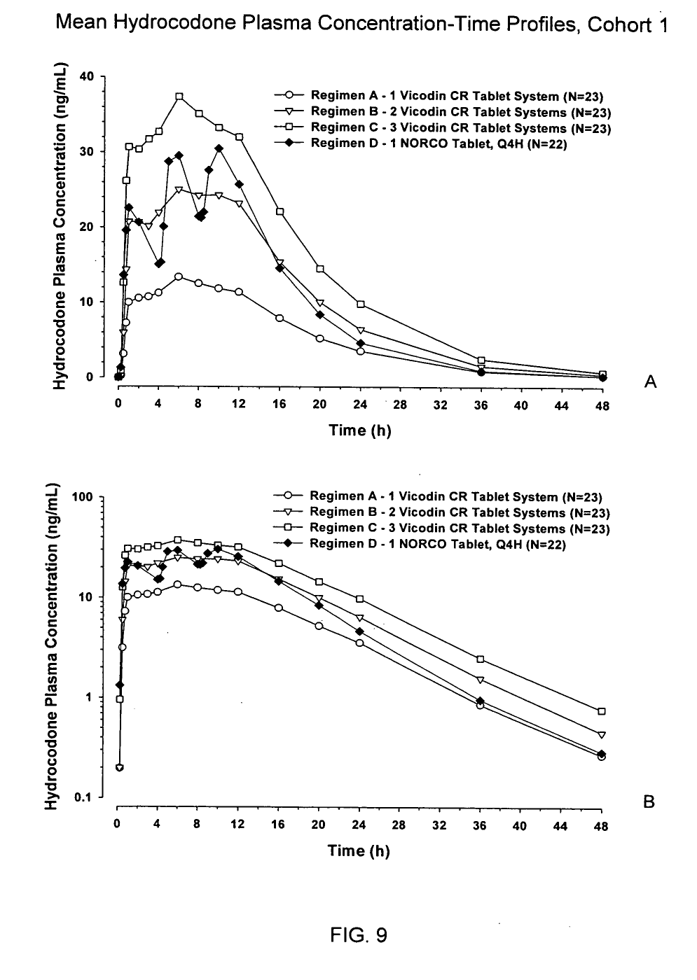

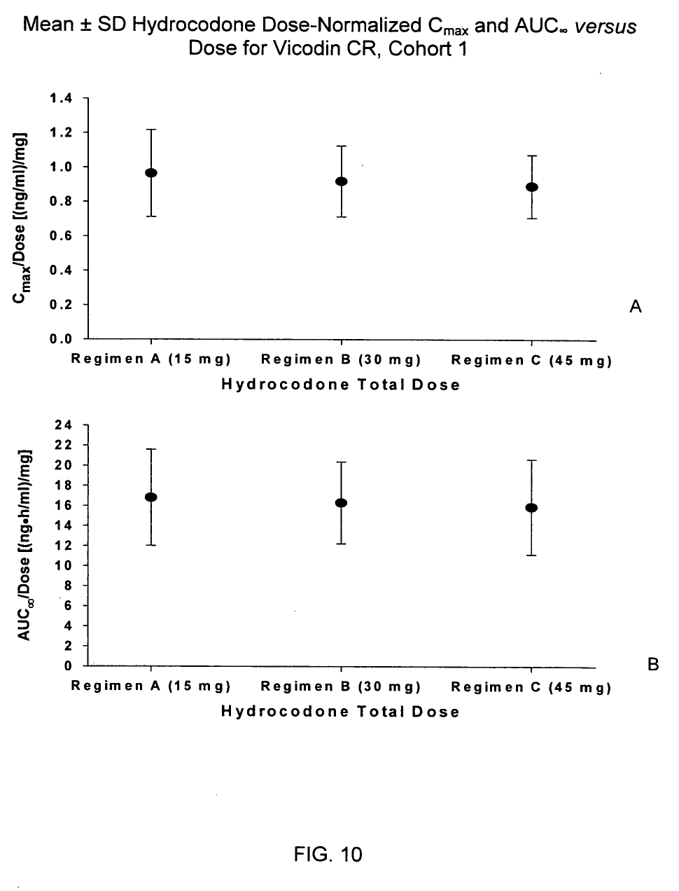

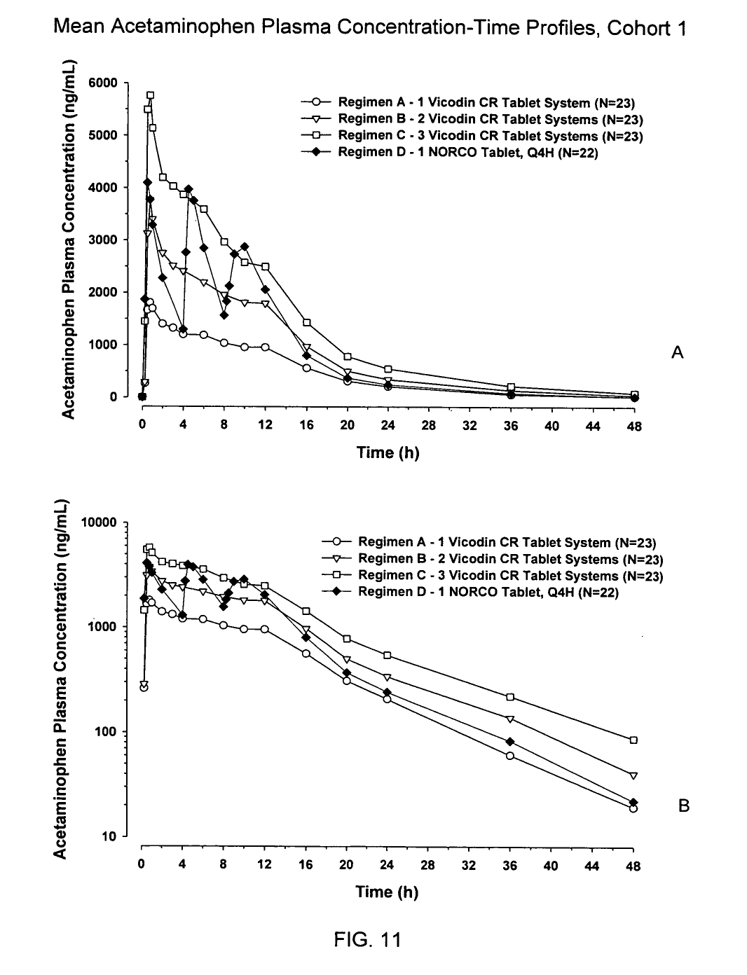

In the second clinical trial, described in Example 6, the sustained release dosage forms of hydrocodone and acetaminophen demonstrated similar results to that observed in the first clinical trial, based on the dosage form having a T₉₀ of 8 hours. FIGS. 9-11 demonstrate the in vivo plasma concentrations of hydrocodone, acetaminophen and hydromorphone, respectively, after administering one, two or three representative dosage forms, in comparison with an immediate release dosage form dosed at zero, four and eight hours. As FIGS. 9 and 10 illustrate, volunteers receiving one to three tablets of the dosage form having a T₉₀ of 8 hours prepared according the procedure of Example 2 exhibited a rapid rise in plasma concentrations of hydrocodone and acetaminophen after oral administration at time zero. The plasma concentrations of hydrocodone and acetaminophen reach an initial peak due to the release of hydrocodone and acetaminophen from the drug coating. Subsequent to the initial release of hydrocodone and acetaminophen, the sustained release of the dosage forms provides for continued release of hydrocodone and acetaminophen to the patient, as demonstrated by the sustained hydrocodone and acetaminophen plasma levels shown in FIGS. 9 and 10. The plasma concentrations of hydromorphone, a metabolite of hydrocodone, are shown in Tables 2-4 discussed above and FIG. 11. As before, the plasma profile for hydrocodone was zero order or ascending at all doses, while the plasma profile for acetaminophen was zero order or descending for all doses. Hydromorphone levels were substantially zero order throughout the dosing interval.

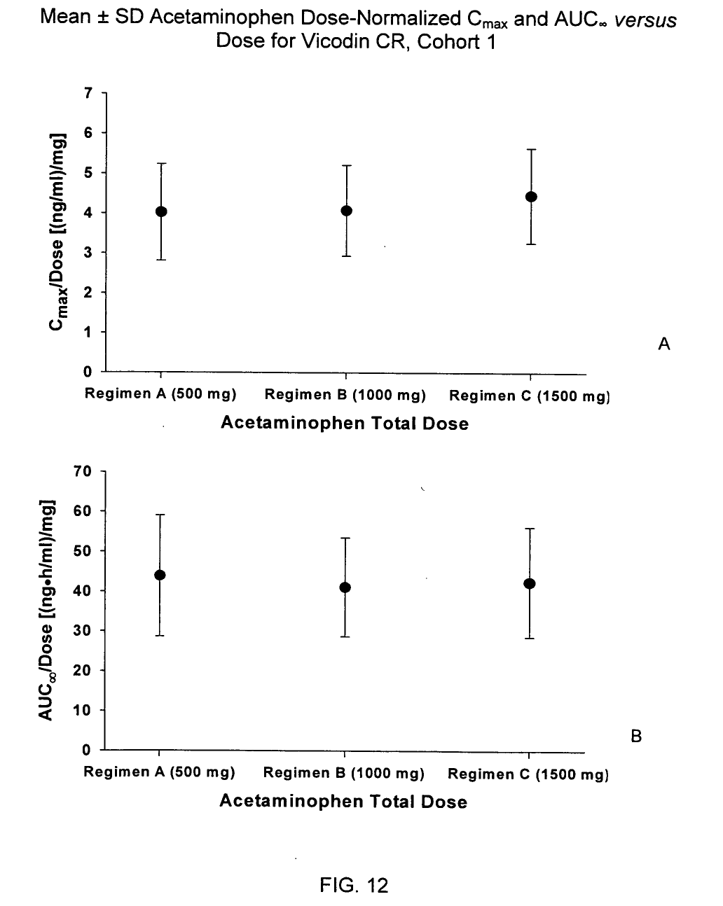

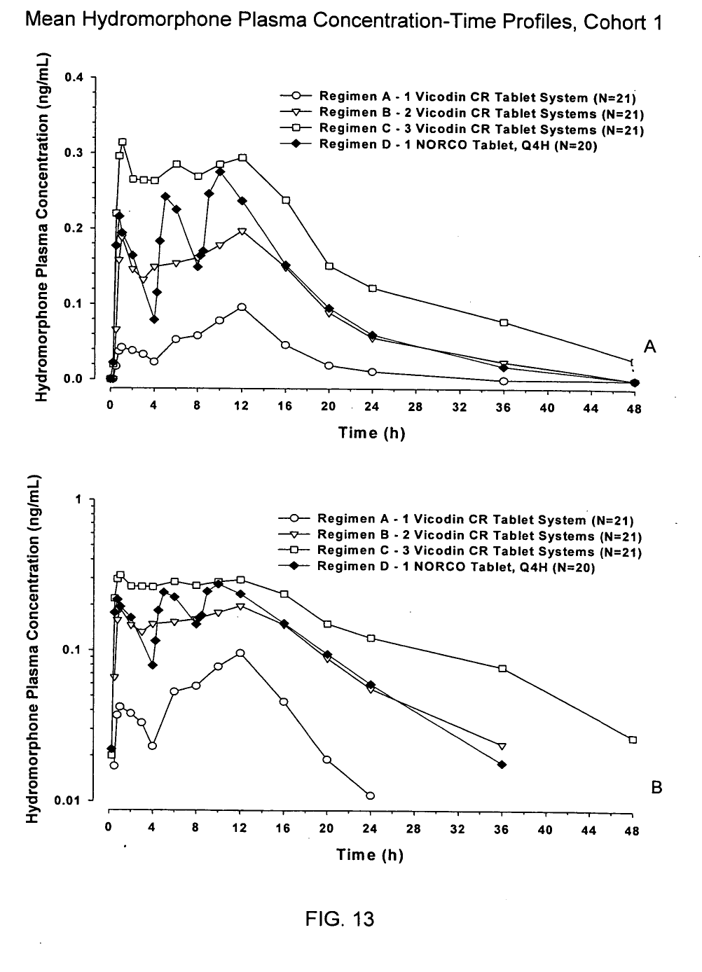

Overall, in the second clinical trial, the sustained release dosage forms of hydrocodone and acetaminophen concentrations were dose proportional across 1, 2 and 3 tablets. For example, FIGS. 12 and 13 illustrate the mean Cmax and AUC_{∞} (±the standard deviation) for the normalized dose of hydrocodone and acetaminophen observed during this trial.

Steady state for the sustained release dosage forms of hydrocodone and acetaminophen Q12H was achieved by 24 hours; no statistically significant monotonic rising time effect was observed in the hydrocodone and acetaminophen trough concentrations measured between 24 and 72 hours. Accumulation was minimal as steady-state peak concentrations of hydrocodone were less than 50% and acetaminophen were less than 25% greater than those achieved following the administration of a single dose. Hydromorphone levels reached steady state during the second day of dosing as the 36 and 72 hours hydromorphone trough concentrations were not statistically significantly different.

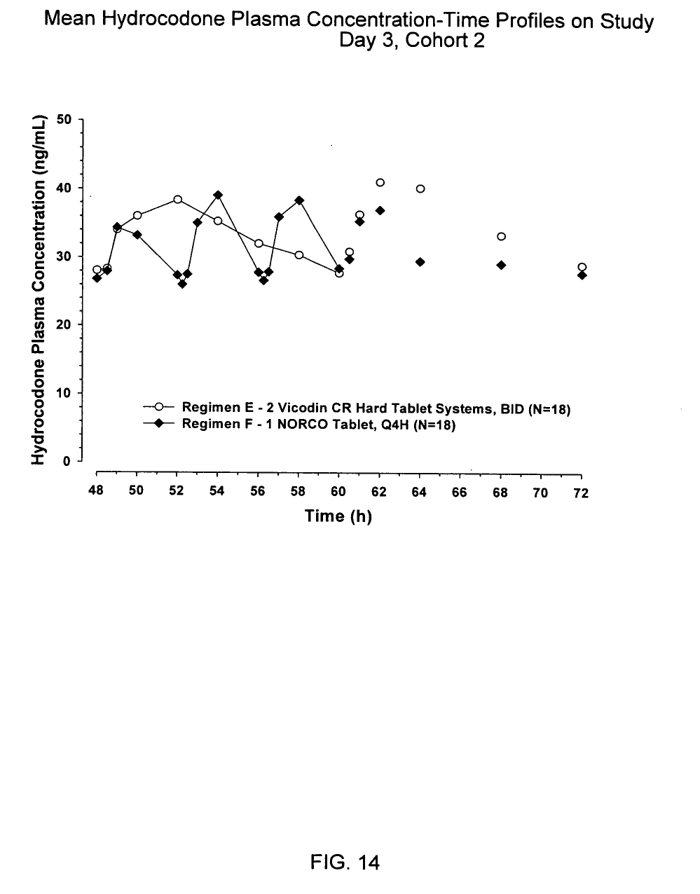

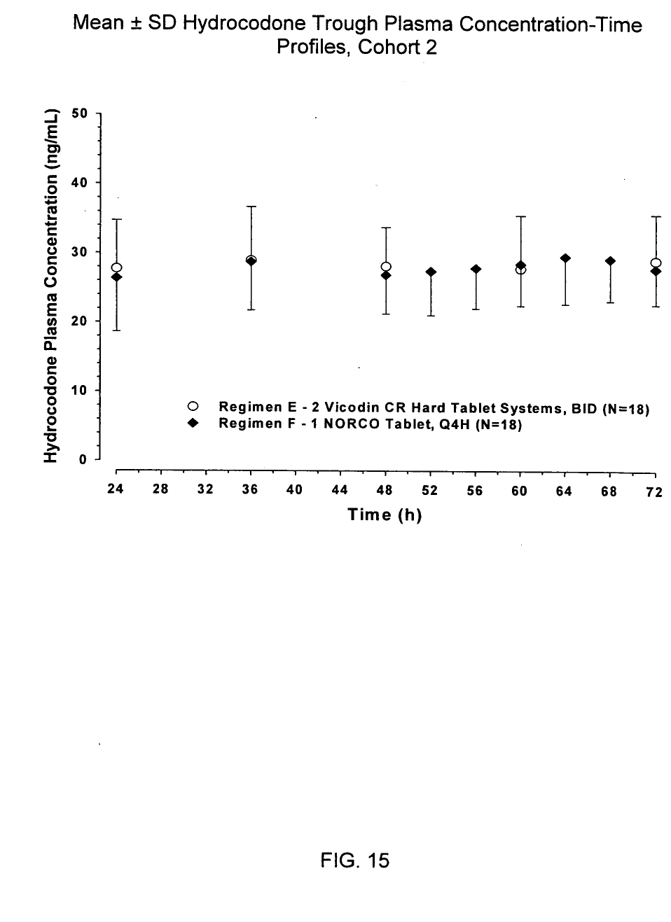

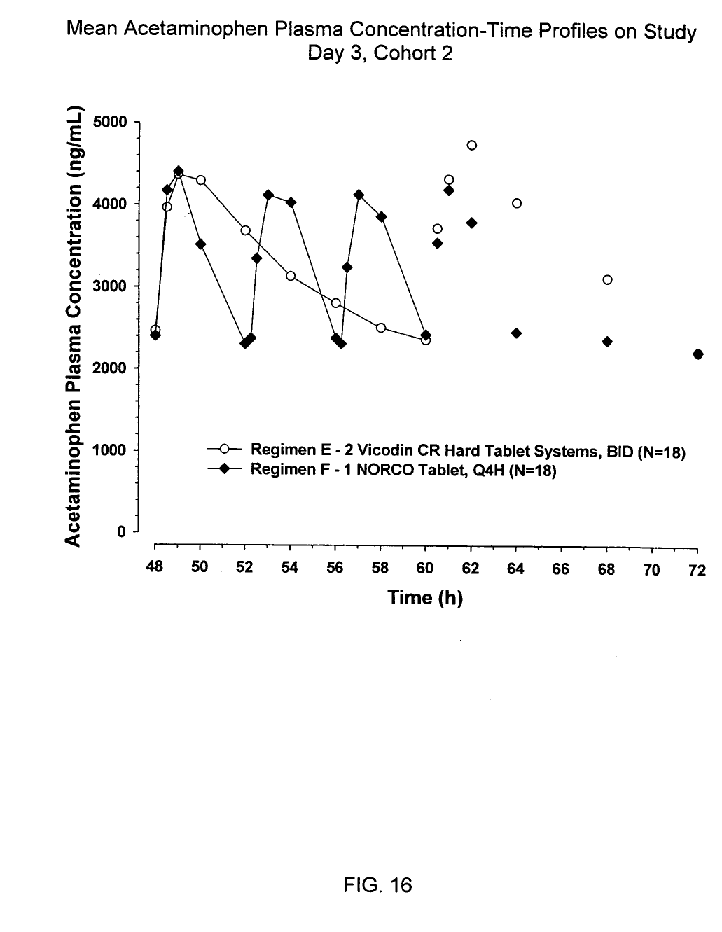

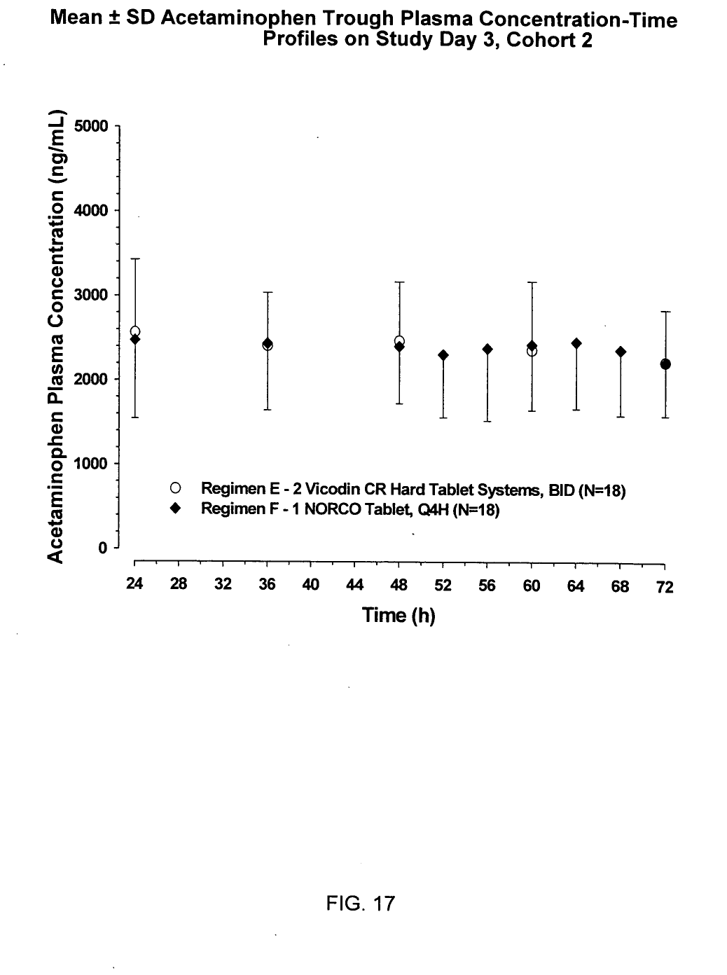

These steady state results are demonstrated in FIGS. 14-17. FIG. 14 illustrates the mean hydrocodone plasma concentration-time profiles at steady state (±the standard deviation) for a representative dosage form dosed every 12 hours and an immediate release dosage form dosed every four hours, while FIG. 15 illustrates the mean hydrocodone trough plasma concentration-time profiles at steady state (±the standard deviation). FIG. 16 illustrates the mean acetaminophen plasma concentration-time profiles at steady state (±the standard deviation) for a representative dosage form dosed every 12 hours and an immediate release dosage form dosed every four hours, while FIG. 17 illustrates the mean acetaminophen trough plasma concentration-time profiles at steady state (±the standard deviation).

The steady state results demonstrate a decreased fluctuation in plasma hydrocodone and acetaminophen when patients were dosed with the sustained release dosage forms in comparison with every 4 hour dosing of an immediate release formulation of hydrocodone and acetaminophen. The results also demonstrate that for hydrocodone the peak concentration is in general less than twice as large as the minimum concentration and that for acetaminophen the peak concentration is in general less than 3.5 times as large as the minimum concentration.

The test Regimen B (single dose of the sustained release dosage forms of hydrocodone and acetaminophen, 2 tablets) was equivalent to reference Regimen D (NORCO®, 1 tablet every 4 hours for 3 doses) with respect to AUC; the 90% confidence intervals for the ratios of AUC central values for hydrocodone and acetaminophen were contained within the 0.80 to 1.25 range. The ratio of the Regimen B to Regimen D Cₘₐₓ central values was estimated to be 0.79 for hydrocodone and 0.81 for acetaminophen, both estimated ratios statistically lower than 1.0. The lower bound of the 90% confidence intervals for the ratios of hydrocodone and acetaminophen Cₘₐₓ central values fell below 0.80. Again, the decrease in Cₘₐₓ while maintaining AUC levels provided by the sustained release dosage forms provides a dosage form that should be less likely to result in adverse events.

The test Regimen E (the sustained release dosage forms of hydrocodone and acetaminophen, 2 tablets Q12H) was equivalent to the reference Regimen F (NORCO,® 1 tablet Q4H) at steady state; the 90% confidence intervals for the ratios of AUC and Cₘₐₓ central values for hydrocodone and acetaminophen were contained within the 0.80 to 1.25 range.

These results demonstrate an improvement in plasma profile provided by the sustained release dosage forms over the immediate release comparator. The ranges in Cₘₐₓ may be helpful to limit the adverse event profile of the opioid combination product while maintaining efficacy. Current immediate release formulations produce higher Cₘₐₓ values, which may be associated with adverse events. Also by limiting the peak concentrations and rate of rising concentration produced by the dosage forms, it may be possible to limit the abuse profile of the combination product, as the same dose of an immediate release product may produce a greater “high” than this product.

The AUC values produced by the sustained release dosage forms are near the lower end of AUC values thought to limit the likelihood of breakthrough pain and adverse events, especially acute liver toxicity. The dosage forms further provide a mean fluctuation of opioid less than about 50%, thus limiting the likelihood of adverse events while maintaining efficacy. It is conventionally thought that if the plasma level is maintained above a minimum level, then the product should be efficacious, and if the ranges of Cₘₐₓ are limited above this level, then the rate of adverse events should be minimized.

For the hydrocodone/acetaminophen combination, to the inventors' knowledge, the relationship between plasma concentration and pharmacodynamic effect has not been previously established, therefore prior to the present studies, there was no certainty what particular plasma concentration profile would result (Cₘₐₓ, Cₘᵢₙ, AUC, DFL (“degree of fluctuation”, or “fluctuation”), Tₘₐₓ, etc.) prior to testing the dosage form in patients. Further, there was no certainty what plasma profile would provide the desired efficacy (pain relief) for a sustained period of time or reduced adverse events. In fact, at least one trial to demonstrate safety and efficacy for a modified release product is required by regulatory bodies where the relationship between plasma concentration and pharmacodynamic effect has not been established for the immediate release product.

An advantage of the present invention relates to the improved ability to treat pain in a variety of patients. Pain management often involves a combination of a 30 chronic pain medication with a rescue medication. The chronic pain medication is used to treat base levels of pain in a patient, and the rescue medication is used to treat breakthrough pain (pain that “breaks through” the level of analgesia provided by the chronic pain medication).

Physicians treating patients for breakthrough pain generally prefer to use the same medication for rescue as is being used for the underlying chronic pain. This is for a variety of reasons, including reducing concerns about drug-drug interactions, convenience in converting rescue medication to the pain therapy, and also conservative management of a patient's overall therapy. In the case of the present invention, a physician administering the inventive dosage forms would prefer to use a dosage form that comprises hydrocodone bitartrate and acetaminophen as rescue medication. In a preferred embodiment, the rescue medication is Vicodin®.

One concern about use of a dosage form comprising hydrocodone bitartrate and acetaminophen as a rescue medication is that there is an upper limit on how much acetaminophen should be administered to a patient over a 24 hour period. That limit is generally accepted to be 4000 mg/day. For example, examining the amount of acetaminophen in a Vicodin® tablet one finds that the weight ratio of acetaminophen to hydrocodone bitartrate is 100:1, with recommended dosing being 1 to 2 tablets every 4 to 6 hours not to exceed 8 tablets in 24 hours. Eight tablets would correspond to 4000 mg/day of acetaminophen. It is clear that for some patients, Vicodin® could not be dosed around the clock without potentially exceeding the 8 tablet per day limit.

Accordingly, in designing a dosage form that comprises hydrocodone bitartrate and acetaminophen for all day pain relief, the inventors recognized that it would be desirable to decrease the amount of base line acetaminophen provided to a patient while still providing for adequate pain relief. The inventors unexpectedly discovered that it was possible to rebalance the amount of hydrocodone bitartrate and acetaminophen so as to have less acetaminophen in the inventive dosage forms and more hydrocodone bitartrate, yet still have efficacy in pain treatment (see Example 7). Accordingly, one reason for the usefulness, novelty and unobviousness of the plasma levels, release rates, methods and dosage forms of hydrocodone bitartrate and acetaminophen disclosed herein is that such levels, rates, methods and dosage forms provide for efficacy with reduced dosing of acetaminophen.

Rebalancing while maintaining efficacy provides an unexpected benefit in that conventional dosage forms comprising hydrocodone bitartrate and acetaminophen can now be used as rescue medication in treatment regimens in combination with the inventive dosage forms described herein while still staying below the recommended daily limit for acetaminophen administration. In this manner, treatment of patients for pain is improved, and represents and advancement in the art.

Accordingly, the dosage forms described herein also provide a method of treating pain comprising administering the sustained release dosage forms described herein, and further comprising administering additional rescue medication to patients in need thereof, in the form of an immediate release formulation, such as acetaminophen or Vicodin®. These methods are contemplated to be useful for managing both acute and chronic pain, depending on the patient's perceived pain, and may be particularly advantageous in the treatment of acute pain, such as postoperative pain. These methods provide an increased safety margin for patients in that baseline pain management is provided utilizing only 1000-3000 mg/day of acetaminophen in the sustained release dosage forms described herein, when dosed as described in Example 5-7. Therefore, the methods of treating pain described herein provide pain relief with greater safety for patients in need of additional rescue medication. In addition, the dosage forms provide a greater safety margin for acetaminophen exposure in the chronic pain setting, even in the absence of rescue medication.

The pharmacokinetic results obtained from both clinical trials are shown in Tables 2-5 below. Table 2 presents the pharmacokinetic parameters of acetaminophen and hydrocodone bitartrate, Table 3 presents the pharmacokinetic parameters calculated per dose of acetaminophen and hydrocodone bitartrate, and Table 4 presents the pharmacokinetic parameters for patients exhibiting plasma profiles characterized by two peak concentrations. Table 5 presents the pharmacokinetic parameters of acetaminophen and hydrocodone bitartrate produced by various dosages of a preferred embodiment.

TABLE 2
Pharmacokinetic parameters of acetaminophen
and hydrocodone bitartrate

| | Study | | |
|---|---|---|---|
| PK parameter | Regimen | Mean ± SD | Min to Max |
| Cmax (ng/mL)- HC | 363A | 33.6 ± 9.2 | 17.2-56.9 |
| | 363B | 29.6 ± 7.4 | 18.4-47.2 |
| | 363C | 26.6 ± 7.2 | 15.6-40.5 |
| | 597B | 25.3 ± 5.7 | 12.7-35.8 |
| Cmax (μg/mL)- APAP | 363A | 5.6 ± 1.9 | 3.2-10.2 |
| | 363B | 5.9 ± 2.0 | 2.7-9.7 |
| | 363C | 5.8 ± 2.1 | 2.0-10.4 |
| | 597B | 4.1 ± 1.1 | 2.3-7.3 |
| Cmax- HM in | 597B | 0.238 ± 0.116 | 0-0.509 |
| nonPM(ng/mL) | | | |
| AUC-HC(ng*hr/mL) | 363A | 393 ± 118 | 228-700 |
| | 363B | 397 ± 122 | 236-710 |
| | 363C | 406 ± 114 | 229-638 |
| | 597B | 449 ± 113 | 266-754 |
| AUC- | 363A | 42.6 ± 11.4 | 25.9-72.2 |
| APAP(μg*hr/mL) | 363B | 42.6 ± 10.3 | 24.7-69.0 |
| | 363C | 45.1 ± 12.0 | 24.9-65.5 |
| | 597B | 41.1 ± 12.4 | 22.5-67.8 |
| AUC- HM in | 597B | 7.5 ± 2.8 | 2.9-12.9 |
| nonPM(ng*hr/mL) | | | |
| C12- HC(ng/mL)- | 363A | 17.3 ± 5.5 | 8.6-28.3 |
| | 363B | 16.4 ± 5.2 | 8.7-28.5 |
| | 363C | 16.3 ± 5.3 | 6.8-27.2 |
| | 597B | 21.3 ± 6.1 | 11.7-31.1 |
| C12- APAP(μg/mL)- | 363A | ±0.4 | 0.5-2.0 |
| | 363B | ±0.5 | 0.5-2.1 |
| | 363C | 1.5 ± 0.5 | 0.8-2.6 |
| | 597B | 1.8 ± 0.7 | 0.7-3.3 |
| C12- HM in | 597B | 0.2 ± 0.12 | 0-0.38 |
| nonPM(ng/mL)- | | | |
| Cmax/C12-HC | 363A | 2.0 ± 0.4 | 1.5-3.3 |
| | 363B | 1.9 ± 0.5 | 3.5-3.3 |
| | 363C | 1.7 ± 0.5 | |
| | 597B | 1.2 ± 0.2 | 1.0-1.8 |
| Cmax/C12-APAP | 363A | 5.6 ± 2.5 | 2.3-14.8 |
| | 363B | 5.3 ± 2.7 | 2.1-11.1 |
| | 363C | 4.1 ± 1.6 | 2.1-8.4 |
| | 597B | 2.6 ± 1.0 | 1.2-5.4 |
| Relative Cmax to IR- | 363A | 0.96 ± 0.21 | 0.63-1.42 |
| HC | 363B | 0.86 ± 0.17 | 0.46-1.17 |
| | 363C | 0.76 ± 0.15 | 0.49-1.03 |
| | 597B | 0.80 ± 0.14 | 0.59-1.05 |
| Relative AUC to IR- | 363A | ±0.18 | 0.83-1.67 |
| HC | 363B | ±0.09 | 0.86-1.17 |
| | 363C | 1.04 ± 0.20 | 0.85-1.87 |
| | 597B | 1.04 ± 0.10 | 0.87-1.23 |
| Relative Cmax to IR- | 363A | 0.9 ± 0.4 | 0.6-1.9 |
| APAP | 363B | ±0.4 | 0.4-1.9 |
| | 363C | 0.9 ± 0.3 | 0.4-1.7 |
| | 597B | 0.8 ± 0.2 | 0.5-1.2 |
| Relative AUC to IR- | 363A | ±0.2 | 0.8-1.7 |
| APAP | 363B | ±0.1 | 0.9-1.2 |
| | 363C | ±0.2 | 0.9-1.7 |
| | 597B | 1.0 ± 0.1 | 0.8-1.2 |
| Tmax-HC | 363A | 4.5 ± 2.6 | 0.75-8 |
| | 363B | 4.3 ± 3.4 | 0.75-8 |
| | 363C | 1.9 ± 2.1 | 0.5-6 |
| | 597B | 6.7 ± 3.8 | 1-12 |
| Tmax- APAP | 363A | 2.8 ± 2.7 | 0.5-6 |
| | 363B | ±1.3 | 0.5-6 |
| | 363C | 0.9 ± 0.8 | 0.5-4 |
| | 597B | 1.1 ± 1.1 | 0.5-4 |
| Tmax- HM | 597B | 7.5 ± 5.6 | 0.5-16 |
| Cmax/AUC-HC | 363A | 0.09 ± 0.01 | 0.07-0.12 |
| | 363B | 0.08 ± 0.01 | 0.05-0.09 |
| | 363C | 0.07 ± 0.01 | 0.05-0.11 |
| | 597B | 0.057 ± 0.008 | 0.043-0.069 |
| Cmax/AUC-APAP | 363A | 0.13 ± 0.04 | 0.08-0.23 |
| | 363B | 0.14 ± 0.05 | 0.08-0.27 |
| | 363C | 0.13 ± 0.05 | 0.07-0.23 |
| | 597B | 0.104 ± 0.028 | 0.057-0.167 |
| Cmax/AUC- HM | 597C | 0.039 ± 0.018 | 0.015-0.092 |
| Peak width, 50- HC | 363A | 10.4 ± 4.0 | 6.2-13.6 |
| | 363B | 11.7 ± 2.8 | 7.5-19.2 |
| | 363C | 13.7 ± 4.9 | 2.1-20.9 |
| | 597B | 16.0 ± 3.6 | 3.5-21 |
| Peak width, 50 - APAP | 363A | 5.5 ± 3.0 | 0.4-9.2 |
| | 363B | 5.0 ± 3.9 | 0.3-13.1 |
| | 363C | 4.5 ± 3.7 | 0.3-11.1 |
| | 597B | 7.6 ± 4.7 | 1.5-14.5 |
| Ratio APAP:HC at 1 | 363A | 199.1 ± 84.9 | 89-419 |
| hour | 363B | 197.5 ± 71.6 | 103-396 |
| | 363C | 183.0 ± 62.7 | 87-318 |
| | 597B | 185.7 ± 44.1 | 118.6-277.5 |
| Ratio APAP:HC at 6 | 363A | 125.1 ± 40.7 | 69-229 |
| hours | 363B | 116.6 ± 33.2 | 55-190 |
| | 363C | 115.2 ± 35.7 | 54-177 |
| | 597B | 95.8 ± 25.0 | 44.6-147.8 |
| Ratio APAP:HC at 12 | 363A | 77.6 ± 41.2 | 26-187 |
| hours | 363B | 83.9 ± 36.7 | 30-170 |
| | 363C | 98.2 ± 36.9 | 38-179 |
| | 597B | 85.0 ± 27.2 | 32.7-146.7 |
| Ctrough, ss- | 597E | 25.5 ± 7.1 | 14.8-43.1 |
| HC(ng/mL)- | | | |
| Ctrough, ss- | 597E | 2.4 ± 0.8 | 1.0-4.3 |
| APAP(μg/mL)- | | | |
| Ctrough, ss- | 597E | 0.54 ± 0.24 | 0.24-0.93 |
| HM(ng/mL)- | | | |
| Cmax, ss- HC(ng/mL) | 597E | 37.0 ± 6.8 | 26.7-50.2 |
| Cmax, ss- | 597E | 5.0 ± 0.9 | 3.6-7.1 |
| APAP(μg/mL) | | | |
| Cmax, ss- HM(ng/mL) | 597E | 0.67 ± 0.28 | 0.27-1.50 |
| Cmin, ss- HC(ng/mL) | 597E | 23.9 ± 5.2 | 13.6-34.1 |
| Cmin, ss- | 597E | 2.2 ± 0.8 | 1.0-3.8 |
| APAP(μg/mL) | | | |
| Cmin, ss- HM(ng/mL) | 597E | 0.43 ± 0.17 | 0.17-0.77 |
| AUCss- HC(ng*hr/mL) | 597E | 368 ± 78 | 251-558 |
| AUCss- | 597E | 38.9 ± 10.9 | 25.3-71.0 |
| APAP(μg*hr/mL) | | | |
| AUCss- | 597E | 6.4 ± 2.4 | 2.7-11.6 |
| HM(ng*hr/mL) | | | |
| Cmax/Cmax ss- | 597E | 137.0 ± 26.5 | 85.2-186.3 |
| APAP:HC | | | |
| Ctrough/Ctrough ss- | 597E | 94.4 ± 28.5 | 48.0-153.1 |
| APAP:HC | | | |
| Ratio APAP:HCss at 1 | 597E | 144.8 ± 39.7 | 96.2-230.6 |
| hour | | | |
| Ratio APAP:HCss at 4 | 597E | 105.8 ± 25.6 | 58.7-157.1 |
| hours | | | |
| Ratio APAP:HCss at 6 | 597E | 96.4 ± 25.9 | 52.1-145.2 |
| hour | | | |
| Cmax/AUCss-HC | 597E | 0.101 ± 0.009 | 0.090-0.120 |
| Cmax/Cminss-HC | 597E | 1.6 ± 0.2 | 1.3-2.2 |
| Cmax/AUCss-APAP | 597E | 0.132 ± 0.027 | 0.100-0.208 |
| Cmax/Cminss-APAP | 597E | 2.5 ± 0.9 | 1.5-5.3 |
| Cmax/AUCss-HM | 597E | 0.105 ± 0.012 | 0.088-0.131 |
| Cmax/Cminss-HM | 597E | 1.6 ± 0.2 | 1.3-2.0 |
| Peak width, 50ss- HC | 597E | >12 | |
| Peak width, 50 ss- | 597E | 8.9 ± 3.2 | 3.5-12.0 |
| APAP | | | |
| Peak width, 50 ss- HM | 597E | >12 | |
| Relative Cmaxss to IR- | 597E | 1.0 ± 0.2 | 0.7-1.3 |
| HC | | | |
| Relative Cmaxss to IR- | 597E | 1.0 ± 0.2 | 0.6-1.4 |
| APAP | | | |
| Relative Cmaxss to IR- | 597E | 1.0 ± 0.3 | 0.5-1.6 |
| HM | | | |
| Relative Ctroughss to | 597E | 1.0 ± 0.2 | 0.7-1.3 |
| IR- HC | | | |
| Relative Ctroughss to | 597E | 1.0 ± 0.1 | 0.7-1.4 |
| IR- APAP | | | |
| Relative Ctroughss to | 597E | 1.1 ± 0.2 | 0.8-1.5 |
| IR- HM | | | |
| Relative AUCss to IR- | 597E | 1.0 ± 0.2 | 0.8-1.6 |
| HC | | | |
| Relative AUCss to IR- | 597E | 1.0 ± 0.1 | 0.7-1.2 |
| APAP | | | |
| Relative AUCss to IR- | 597E | 1.0 ± 0.2 | 0.7-1.4 |
| HM | | | |
| Fluctuation- HC | 597E | 43.6 ± 14.2 | 24.7-76.2 |
| Fluctuation- APAP | 597E | 92.7 ± 39.3 | 45.7-201.9 |
| Fluctuation- HM | 597E | 46.3 ± 14.5 | 22.7-75.3 |

TABLE 3
Pharmacokinetic parameters calculated per dose
of acetaminophen and hydrocodone bitartrate*

| | Study | | |
|---|---|---|---|
| PK parameter | Regimen | Mean ± SD | Min to Max |
| Cmax/Dose | 363A | ±0.3 | 0.6-1.9 |
| (ng/mL/mg)- HC | 363B | ±0.3 | 0.6-1.6 |
| | 363C | 0.9 ± 0.2 | 0.5-1.4 |
| | 597A | 0.9 ± 0.2 | 0.5-1.5 |
| | 597B | 0.8 ± 0.2 | 0.4-1.2 |
| | 597C | 0.8 ± 0.2 | 0.4-1.1 |
| Cmax/Dose | 363A | 5.6 ± 1.9 | 3.2-10.2 |
| (ng/mL/mg)- APAP | 363B | 5.9 ± 2.0 | 2.8-9.7 |
| | 363C | 5.8 ± 2.1 | 10.4 |
| | 597A | 4.0 ± 1.2 | 7.0 |
| | 597B | 4.1 ± 1.1 | 2.3-7.3 |
| | 597C | 4.5 ± 1.2 | 2.1-6.4 |
| AUC/Dose- | 363A | 13.1 ± 3.9 | 7.6-23.3 |
| HC(ng*hr/mL/mg) | 363B | 13.2 ± 4.1 | 7.9-23.7 |
| | 363C | 13.5 ± 3.8 | 7.6-21.3 |
| | 597A | 15.5 ± 4.4 | 9.1-25.4 |
| | 597B | 15.0 ± 3.7 | 8.9-25.1 |
| | 597C | 14.6 ± 4.4 | 7.0-26.2 |
| AUC/Dose-APAP | 363A | 42.6 ± 11.4 | 25.9-72.2 |
| (ng*hr/mL/mg) | 363B | 42.6 ± 10.3 | 24.7-69.0 |
| | 363C | 45.1 ± 12.0 | 25.0-65.5 |
| | 597A | 43.9 ± 15.2 | 18.4-79.9 |
| | 597B | 41.1 ± 12.4 | 22.5-67.8 |
| | 597C | 42.4 ± 13.8 | 21.0-73.8 |

(Cmax is ng/mL and AUC is ng*hr/mL per mg hydrocodone bitartrate administered and Cmax is μg/mL or AUC is μg*hr/mL per mg acetaminophen administered)

TABLE 4
Pharmacokinetic parameters for patients exhibiting plasma
profiles characterized by two peak concentrations

| | Study | | |
|---|---|---|---|
| PK parameter | Regimen | Mean ± SD | Min to Max |
| Cmax1(ng/mL)- HC | 363A | 26.2 ± 8.5 | 12.1-41.7 |
| | 363B | 25.6 ± 9.8 | 5.4-41.7 |
| | 363C | 25.2 ± 7.7 | 13.5-40.5 |
| | 597B | 21.7 ± 4.5 | 12.0-32.3 |
| Tmax1 (hr) HC | 363A | ±1.3 | 0.5-6 |
| | 363B | ±0.4 | 0.75-2 |
| | 363C | 0.9 ± 0.5 | 0.5-3 |
| | 597B | 1.6 ± 0.9 | 1-4 |
| Cmin (ng/ml) HC | 363A | 18.4 ± 5.1 | 11.0-30.9 |
| | 363B | 16.2 ± 6.1 | 5.2-28.0 |
| | 363C | 16.0 ± 4.8 | 8.9-27.1 |
| | 597B | 18.0 ± 4.8 | 9.0-30.8 |
| Cmax2 (ng/ml) HC | 363A | 30.8 ± 9.7 | 17.2-56.9 |
| | 363B | 26.7 ± 7.7 | 15.4-47.2 |
| | 363C | 22.4 ± 6.2 | 12.8-32.3 |
| | 597B | 24.7 ± 6.1 | 12.7-34.8 |
| Tmax2 (hr) HC | 363A | 5.4 ± 1.5 | 8 |
| | 363B | 6.5 ± 1.9 | 8 |
| | 363C | 5.6 ± 2.7 | 16 |
| | 597B | 9.0 ± 2.4 | 6-12 |
| Cmax1(μg/mL)- APAP | 363A | 5.1 ± 2.1 | 2.4-10.2 |
| | 363B | 5.5 ± 2.1 | 1.6-9.7 |
| | 363C | 5.7 ± 2.2 | 10.4 |
| | 597B | 4.1 ± 1.2 | 2.1-7.3 |
| Tmax1 (hr) APAP | 363A | ±0.8 | 0.5-3 |
| | 363B | 0.8 ± 0.3 | 0.5-2 |
| | 363C | 0.8 ± 0.5 | 0.5-3 |
| | 597B | 0.7 ± 0.2 | 0.5-1.0 |
| Cmin (μg/mL) APAP | 363A | ±0.8 | 1.6-4.3 |
| | 363B | 2.3 ± 0.9 | 0.7-3.8 |
| | 363C | 2.2 ± 0.9 | 0.8-4.5 |
| | 597B | 2.0 ± 0.8 | 0.7-4.1 |
| Cmax2 (μg/mL) | 363A | 4.2 ± 1.4 | 2.6-8.8 |
| APAP | 363B | ±1.1 | 5.8 |
| | 363C | 2.7 ± 1.0 | 4.6 |
| | 597B | 2.4 ± 0.9 | 1.0-4.1 |
| Tmax2 (hr) APAP | 363A | 4.6 ± 1.9 | 1-8 |
| | 363B | 5.7 ± 3.4 | 16-16 |
| | 363C | 6.1 ± 4.4 | |
| | 597B | 7.7 ± 4.2 | 2.0-16.0 |

TABLE 5
Pharmacokinetic parameters of acetaminophen
and hydrocodone bitartrate from Example 6

| | Study | | |
|---|---|---|---|
| PK parameter | Regimen | Mean ± SD | Min to Max |
| Cmax (ng/mL)- HC | 597A | 13.3 ± 3.5 | 7.9-21.8 |
| | 597B | 25.3 ± 5.7 | 12.7-35.8 |
| | 597C | 36.8 ± 7.6 | 19.9-48.7 |
| Cmax (μg/mL)- APAP | 597A | 2.0 ± 0.6 | 3.5 |
| | 597B | 4.1 ± 1.1 | 2.3-7.3 |
| | 597C | 6.7 ± 1.8 | 3.2-9.6 |
| AUC-HC(ng*hr/mL) | 597A | 232 ± 66 | 137-382 |
| | 597B | 449 ± 113 | 266-754 |
| | 597C | 658 ± 197 | 313-1180 |
| AUC- | 597A | 21.9 ± 7.6 | 9.2-40.0 |
| APAP(μg*hr/mL) | 597B | 41.1 ± 12.4 | 22.5-67.8 |
| | 597C | 63.6 ± 20.7 | 31.5-110.7 |
| C12-HC(ng/mL)- | 597A | 10.5 ± 4.0 | 4.2-21.8 |
| | 597B | 21.3 ± 6.1 | 11.7-31.1 |
| | 597C | 29.5 ± 9.1 | 11.7-47.1 |
| C12- APAP(μg/mL)- | 597A | ±0.4 | 0.4-2.0 |
| | 597B | 1.8 ± 0.7 | 0.7-3.3 |
| | 597C | 2.5 ± 1.1 | 0.7-4.7 |

The sustained release hydrocodone and acetaminophen formulations produce plasma profiles of hydrocodone and its metabolite hydromorphone and acetaminophen as presented in the tables above. Preferred aspects are described in the paragraphs that follow. In additional aspects, the sustained release hydrocodone and acetaminophen formulations are also characterized by additional pharmacokinetic values set forth in the above tables. Such pharmacokinetic values may be derived in part based on parameters such as Csteady state, max (ng/ml); Csteady state, min (ng/ml); Ct, min (ng/ml); t steady state, max (hr); ratios of Cmax, AUC, etc. obtained with the sustained release formulation relative to the immediate release comparator; fluctuation (%) (expressed as the difference between Csteady state, max and Csteady state, min expressed as a percentage of Csteady state, min); Tsteady state (days), and combinations thereof.

The sustained release formulations described herein provide a means for producing or providing these plasma profiles in human patients. Any and all of these pharmacokinetic parameters are expressly encompassed within the scope of the invention and the appended claims.

In preferred embodiments, the plasma concentration profile in a patient is characterized by a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose. The plasma concentration profile is further characterized by a minimum Cmax for hydrocodone of about 0.4 ng/mL/mg and a maximum Cmax for hydrocodone of about 1.9 ng/mL/mg and a minimum Cmax for acetaminophen of about 2.0 μg/mL/mg and maximum Cmax for acetaminophen of about 10.4 ng/mL/mg after a single dose. The plasma concentration profile is also characterized by a Cmax for hydrocodone of about 0.8±0.2 ng/mL/mg and a Cmax for acetaminophen of about 4.1±1.1 μg/mL/mg after a single dose.

The plasma concentration profile for hydrocodone is characterized by a Tmax for hydrocodone of about 1.9±2.1 to about 6.7±3.8 hours after a single dose. The plasma concentration profile for hydrocodone is further characterized by a Tmax for hydrocodone of about 4.3±3.4 hours after a single dose. The plasma concentration profile for hydrocodone is also characterized by a Tmax for hydrocodone of about 6.7±3.8 hours after a single dose.

The plasma concentration profile is characterized by a Tmax for acetaminophen of about 0.9±0.8 to about 2.8±2.7 hours after a single dose. The plasma concentration profile is further characterized by a Tmax for acetaminophen of about 1.2±1.3 hours after a single dose.

The dosage form produces a plasma concentration profile characterized by an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose. The plasma concentration profile is further characterized by a minimum AUC for hydrocodone of about 7.0 ng*hr/mL/mg to a maximum AUC for hydrocodone of about 26.2 ng*hr/mL/mg and a minimum AUC for acetaminophen of about 18.4 ng*hr/mL/mg and maximum AUC for acetaminophen of 79.9 ng*hr/mL/mg after a single dose. The plasma concentration profile is also characterized by an AUC for hydrocodone of about 15.0±3.7 ng*hr/mL/mg and an AUC for acetaminophen of 41.1±12.4 ng*hr/mL/mg after a single dose.

The dosage form produces a plasma concentration profile characterized by a Cmax for hydrocodone of between about 0.6 ng/mL/mg to about 1.4 ng/mL/mg and a Cmax for acetaminophen of between about 2.8 ng/mL/mg and 7.9 ng/mL/mg, and by an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

The dosage form produces a plasma concentration profile characterized by a Cmax for hydrocodone of between about 19.4 and 42.8 ng/ml after a single dose of 30 mg hydrocodone. The plasma concentration profile is characterized by a minimum Cmax for hydrocodone of about 12.7 ng/ml and a maximum Cmax for hydrocodone of about 56.9 ng/mL after a single dose of 30 mg hydrocodone. The plasma concentration profile is further characterized by a Cmax for hydrocodone of between about 25.3±5.7ng/ml after a single dose of 30 mg hydrocodone.

The dosage form produces a plasma concentration profile characterized by a Cmax for acetaminophen of between about 3.0 and about 7.9 μg/ml after a single dose of 1000 mg acetaminophen. The plasma concentration profile is characterized by a minimum Cmax for acetaminophen of about 2.0 μg/ml and a maximum Cmax of about 10.4 μg/ml after a single dose of 1000 mg acetaminophen. The plasma concentration profile is further characterized by a Cmax for acetaminophen of between about 4.1±1.1 μg/ml after a single dose of 1000 mg acetaminophen.

The sustained release dosage form produces a plasma concentration profile characterized by an area under the concentration time curve between about 275 and about 562 ng*hr/ml after a single dose of 30 mg hydrocodone bitartrate. The plasma concentration profile is characterized by a minimum area under the concentration time curve of about 228 ng*hr/ml and a maximum area under the concentration time curve of about 754 ng*hr/ml after a single dose of 30 mg hydrocodone bitartrate. The plasma concentration profile is further characterized by an area under the concentration time curve between about 449±113 ng*hr/ml after a single dose of 30 mg hydrocodone bitartrate.

The dosage form produces a plasma concentration profile characterized by an area under the concentration time curve for acetaminophen between about 28.7 and about 57.1 μg*hr/ml after a single dose of 1000 mg acetaminophen. The plasma concentration profile is characterized by a minimum area under the concentration time curve for acetaminophen of about 22.5 μg*hr/ml and a maximum area under the concentration time curve of about 72.2 μg*hr/ml after a single dose of 1000 mg acetaminophen. The plasma concentration profile is further characterized by an area under the concentration time curve for acetaminophen between about 41.1±12.4 μg*hr/ml after a single dose of 1000 mg acetaminophen.

The dosage form produces a plasma concentration profile characterized by a Cmax for hydromorphone of between about 0.12 and about 0.35 ng/ml after a single dose of 30 mg hydrocodone to a non-poor CYP2D6 metabolizer human patient.

The plasma concentration for hydrocodone at 12 hours (C12) is between about 11.0 and about 27.4 ng/ml after a single dose of 30 mg hydrocodone bitartrate in a human patient. The plasma concentration for acetaminophen at 12 hours (C 12) is between about 0.7 and 2.5 μg/ml after a single dose of 1000 mg acetaminophen in a human patient.

The dosage form produces a plasma concentration profile characterized by a width at half height value for hydrocodone of between about 6.4 and about 19.6 hours. The plasma concentration profile is characterized by a width at half height value for acetaminophen of between about 0.8 and about 12.3 hours.

The dosage form produces a plasma concentration profile characterized by a weight ratio of acetaminophen to hydrocodone between about 114.2 and 284 at one hour after oral administration of a single dose containing 1000 mg acetaminophen and 30 mg hydrocodone to a human patient. The plasma concentration profile is characterized by a weight ratio of acetaminophen to hydrocodone between about 70.8 and 165.8 at six hours after oral administration of a single dose containing 1000 mg acetaminophen and 30 mg hydrocodone to a human patient. The plasma concentration profile is further characterized by a weight ratio of acetaminophen to hydrocodone between about 36.4 and 135.1 at 12 hours after oral administration of a single dose containing 1000 mg acetaminophen and 30 mg hydrocodone to a human patient.

In many patients, though not all, certain embodiments of the dosage form produce a plasma concentration profile for hydrocodone characterized by a first peak concentration (Cmax1) occurring within about 1 to 2 hours after oral administration and a second peak concentration (Cmax2), occurring from about 5 to about 9 hours after oral administration to the human patient. Such embodiments of the dosage form produce a plasma concentration profile for acetaminophen characterized by a first peak concentration (Cmax1) occurring within about 1 hour after oral administration and a second peak concentration (Cmax2), occurring from about 4 to about 8 hours after oral administration to the human patient. The plasma concentration profile for hydrocodone is characterized by a first peak concentration occurring at a time Tmax1 occurring from about 0.4 to about 2.5 hours after oral administration and a second peak concentration occurring at a time Tmax2 occurring from about 2.9 to about 11.4 hours after oral administration to the human patient. The plasma concentration profile for hydrocodone is characterized by a first peak concentration occurring at a time Tmax1 occurring from about 1.6±0.9 hours after oral administration and a second peak concentration occurring at a time Tmax2 occurring from about 9.0±2.4 hours after oral administration to the human patient. The dosage form produces a plasma concentration profile for acetaminophen characterized by a first peak concentration occurring at a time Tmax1 occurring within about 0.5 to about 1.8 hours after oral administration and a second peak concentration occurring at a time Tmax2 occurring from about 1.7 to about 11.9 hours after oral administration to the human patient. The plasma concentration profile for acetaminophen is characterized by a first peak concentration occurring at a time Tmax1 occurring within about 0.7±0.2 hours after oral administration and a second peak concentration occurring at a time Tmax2 occurring from about 7.7±4.2 hours after oral administration to the human patient.

The dosage form can produce a plasma concentration profile for hydrocodone further characterized by a minimum concentration (Cmin) between Cmax1 and Cmax2 after oral administration to the human patient. The Cmax1 for hydrocodone is from about 15.8 ng/mL to about 35.4 ng/mL. The minimum Cmax1 for hydrocodone is about 5.4 ng/mL and the maximum Cmax1 is about 41.7 ng/mL. The Cmax2 for hydrocodone is from about 16.2 ng/mL to about 40.5 ng/mL. The minimum Cmax2 for hydrocodone is about 12.7 ng/mL and the maximum Cmax2 is about 56.9 ng/mL. The Cmin for hydrocodone is from about 10.1 ng/mL to about 23.5 ng/mL. The minimum Cmin for hydrocodone is about 5.2 ng/mL and the maximum Cmin is about 30.9 ng/mL.

The dosage form can produce a plasma concentration profile for acetaminophen further characterized by a minimum concentration (Cmin) between Cmax1 and Cmax2 after oral administration to the human patient. The Cmax1 for acetaminophen is from about 2.9 μg/mL to about 7.9 μg/mL. The minimum Cmax1 for acetaminophen is about 1.6 μg/mL and the maximum Cmax1 is about 10.2 μg/mL. The Cmax2 for acetaminophen is from about 1.5 μg/mL to about 5.6 μg/mL. The minimum Cmax2 for acetaminophen is about 1.0 μg/mL and the maximum Cmax2 is about 8.8 μg/mL. The Cmin for acetaminophen is from about 1.2 μg/mL to about 3.8 μg/mL. The minimum Cmin for acetaminophen is about 0.7 μg/mL and the maximum Cmin is about 4.5 μg/mL.

In an acute pain study, a clinical trial was conducted to test the efficacy of a dosage form described in Example 2 in patients undergoing bunionectomy. The pharmacokinetics of hydrocodone and acetaminophen observed in this study were similar to those described in the initial two pharmacokinetic studies described in Examples 5 and 6, and tabulated above. The results of the acute pain study are presented in Example 7.

The efficacy of treatment regimens consisting of administering one tablet, two tablets or placebo tablets to patients was determined as described herein. The sum of pain intensity (SPI) was assessed for each 12-hour period following each dose of study drug (i.e., five 12-hour post dose periods). Based on both the categorical and VAS scores, statistically significant differences were observed between placebo and the one tablet (15 mg hydrocodone bitartrate/500 mg acetaminophen) treatment regimens during the first 2 post dose periods and between placebo and the two tablet (30 mg hydrocodone bitartrate/1000 mg acetaminiophen) treatment regimens during all 5 periods, with lower mean scores (indicating less pain) in patients receiving the sustained release dosage forms. A summary of the sum of pain intensity scores (categorical and VAS) following each of the 5 doses of study drug is presented in Table 15 in Example 7.

In summary, the formulation showed excellent in vivo efficacy (pain relief) in a post-operative setting. In addition, the formulation provided effective plasma concentrations of hydrocodone bitartrate and acetaminophen over a 12-hour period, and exhibited decreased plasma fluctuations (peaks and valleys) than provided by a comparable immediate release formulation, thereby providing plasma concentrations of analgesic agents effective to provide pain relief that are relatively constant over time. Such constant and effective concentrations of analgesic agents provide the potential for greater pain relief when compared to a comparable dose of an immediate release formulation that does not maintain plasma concentrations of analgesic agents in a constant and effective range of plasma concentrations. In addition, such constant and effective concentrations of analgesic agents provide the potential for effective pain relief using a smaller amount of analgesic agents, and further provides increased safety, in comparison with comparable immediate release analgesic formulation. Finally, there is the likelihood of greater patient compliance with the prescribed dosage regimen due to the consistent pain relief as well as the convenience of twice a day dosing

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, pharmaceutical formulations, and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should he accounted for. Unless indicated otherwise, temperature is in degrees ° C. and pressure is at or near atmospheric. All solvents were purchased as HPLC grade, and all reactions were routinely conducted under an inert atmosphere of argon unless otherwise indicated. Unless otherwise indicated, the reagents used were obtained from the following sources: organic solvents, from Aldrich Chemical Co., Milwaukee, Wis.; gases, from Matheson, Secaucus, N.J.

Abbreviations:

APAP: acetaminophen
HBH: hydrocodone bitartrate
HC: hydrocodone
HEC: hydroxyethylcellulose
HM: hydromorphone
HPMC: hydroxypropylmethylcellulose
HPC: hydroxypropylcellulose
PEO: poly(ethylene oxide)
PVP: polyvinylpyrrolidone
PR Pain Relief
TOTPAR Total Pain Relief
PI Pain Intensity
SPI Sum of Pain Intensity

### EXAMPLE 1

A dosage form containing 500 mg acetaminophen and 15 mg hydrocodone was prepared using procedures as follows:

Preparation of the Drug Layer Granulation

A twenty five kilogram lot of the drug layer was granulated using the medium fluid bed granulator (mFBG). A 5% manufacturing excess of hydrocodone bitartrate (HBH) was added to maintain target drug amounts in the compressed cores as established during the experimental scale up work. The binder solution was prepared by dissolving the povidone in purified water making a 7.5 wt % solution.

The specified amounts of APAP, polyethylene oxide 200 K (polyox N-80), croscarmellose sodium (Ac-di-sol), and poloxamer 188 were charged into the FBG bowl. The bed was fluidized and the binder solution was sprayed immediate thereafter. After 1000 g of the binder solution had been metered into the bowl, the granulation process was stopped the preweighed HBH was then charged into the bowl by placing it in a hole in the granulation and covering it up. The technique was employed to minimize the amount of drug that was lost through the filter bags. After a predetermined amount of binder solution had been sprayed, the spray was turned off and the granulation was dried until target moisture content was achieved. The granulation was then milled using a Fluid Air Mill fitted with a 10-mesh screen and using 2250-rpm milling rate.

Milled BHT was then added to replace the BHT lost from the polyethylene oxide and poloxamer in the granulation during processing. BHT is required in the polyethylene oxide and poloxamer to maintain viscosity. The raw material was hand sieved through a 40-mesh screen. The appropriate amount of BHT was dispersed into the top of the granulation in the blender using the Gemco blender, the mixture was blended fro 10 minutes, followed by the blending of the stearic acid and magnesium stearate in the granulation, using the same blender for 1 minute. The stearic acid and magnesium stearate were sized through a 40-mesh screen before being blended to the material in the blender. They were added to facilitate the ejection of the cores from the dies during core compression.

Preparation of the Osmotic Push Layer Granulation

Agglomerates of sodium chloride (NaCl) and ferric oxide were milled through the Quadro Comil fitted with a 21 -mesh screen. The specified amounts of polyethylene oxide, milled NaCl, and milled ferric oxide were layered into the tote. Approximately half of the polyethylene oxide was on the bottom and the rest of the materials were in the middle. The remaining polyethylene oxide was on top. This sandwiching effect prevents the NaCl from re-agglomerating. Povidone was dissolved in purified water to make a binder solution with 13% solids. The appropriate amount of binder solution was prepared to make the granulation.

The dry ingredients in the tote were charged into the FBG bowl. The bed was fluidized, and the binder solution was sprayed as soon as the desired inlet air temperature was achieved. The fluidization airflow was increased by 500 m³/h for approximately every 3 minutes of spraying until the maximum airflow of 4000³/h was reached. After a predetermined amount of binder solution had been sprayed (48.077 kg), the spray was turned off and the granulation was dried to the target moisture content. The granulation was then milled into a 1530 L tote using a Fluid Air Mill fitted with a 7-mesh screen.

Milled BHT was added to prevent degradation of the polyethylene oxide and poloxamer granulation. The raw material was hand sieved through a 40-mesh screen. The appropriate amount of BHT was then dispersed into the top of the granulation in the tote. Using a tote tumbler, the mixture was blended for 10 minutes at 8 rpm, followed by the blending of the stearic acid in the granulation using a tote tumbler for 1 minute at 8 rpm. The stearic acid was sized through a 40-mesh screen before being blended to the material in the tote. It was added to facilitate the ejection of the tablets from the dies during compression.

Bilayer Core Compression

The drug layer granulation and the osmotic push granulation were compressed into bilayer cores using standard compression procedures. The Korsch press was used to manufacture the bilayer longitudinally compressed tablets (LCT). The press was set up with ¼ inch LCT punches and dies with round, deep concave punches and dies. The granulations were scooped into the hoppers leading to the appropriate location or station in the press. The appropriate amount of the drug layer granulation was added to the dies and was lightly tamped on the first compression station of the press. The push granulation was then added and the tablets were compressed to the final tablet thickness under the main compression roll on the second station of the press.

The initial adjustment of the tableting parameters (drug layer) is performed to produce cores with a uniform target drug layer weight of 413 mg containing typically 330 mg of APAP and 10 mg hydrocodone in each tablet. The second layer adjustment (osmotic push layer) of the tableting parameters is performed which bonds the drug layer to the osmotic layer to produce cores with a uniform final core weight, thickness, hardness, and friability. The foregoing parameters can be adjusted by varying the fill space and/or the force setting.

To control the tablet weight, the press has an automatic fill controller, based on compression force, which adjusts the fill quantity of granulation by changing the fill depth in the dies. The compression force and press speed were adjusted as necessary to manufacture tablets with satisfactory properties. The drug layer target weight was 413 mg and the push layer target weight was 138 mg. The pre-compression force was 60 N, adjusted as necessary to obtain quality cores, and the final compression was 6000 N, also adjusted as necessary. The press speed was 13 rpm and there were 14 stations.

Preparation of the Subcoat Solution and Subcoated System

The compressed cores were coated to a target subcoat weight of 17 mg/core. The subcoating solution contained 6 wt % solids and was prepared in a stainless steel mixing vessel. The solids (95% hydroxyethyl cellulose NF and 5% polyethylene glycol 3350) were dissolved in 100% water. The appropriate amount of water was first transferred into the mixing vessel. While mixing the water, the appropriate amount of polyethylene glycol was charged into the mixing vessel followed by the hydroxyethylcellulose. The materials were mixed together in the vessel until all the solids were dissolved.

A Vector Hi-Coater was used for the coating procedure. The coater was started, and after the target exhaust temperature was attained, the bilayer cores (nominally 9 kg per lot) were placed into the coater. The coating solution was sprayed immediately thereafter onto the rotating tablet bed. At regular intervals throughout the coating process, the weight gain was determined. When the desired wet weight gain was achieved (17 mg per core), the coating process was stopped.

Preparation of the Rate Controlling Membrane and Membrane Coated System

The membrane coating solution contained cellulose acetate 398-10 and poloxamer 188 in varying proportions to obtain a desired water permeation rate into the bilayer cores, and was coated onto the cores to a desired weight gain as described in A, B and C below. Weight gain may be correlated with T₉₀ for membranes of varying thickness in the release rate assay. When a sufficient amount of solution has been applied, conveniently determined by attainment of the desired membrane weight gain for a desired T₉₀, the membrane coating process was stopped.

The coating solution contained 5 wt % solids and was prepared in a 20 gallon closed jacketed stainless steel mixing vessel. The solids (75% cellulose acetate 398-10 and 15% poloxamer 188 described in A and B below, for dosage forms having T₉₀s of 6 or 8 hours, or 80% cellulose acetate 398-10 and 20% poloxamer 188, for dosage forms having T₉₀s of 10 hours, described in C below, both containing trace amounts of BHT, 0.0003%) were dissolved in a solvent that consisted of 99.5% acetone and 0.5% water (w/w) and the appropriate amount of acetone and water were transferred into the mixing vessel. While mixing, the vessel was heated to 25° C. to 28° C. and then the hot water supply was turned off. The appropriate amount of poloxamer 188, cellulose acetate 398-10 and BHT were charged into the mixing vessel containing the preheated acetone/water solution. The materials were mixed together in the vessel until all the solids were dissolved.

The subcoated bilayer cores (approximately 9 kg per lot) were placed into a Vector Hi-Coater. The coater was started and after the target exhaust temperature was attained, the coating solution was sprayed onto the rotating tablet bed. At regular intervals throughout the coating process, the weight gain was determined. When the desired wet weight gain was achieved, the coating process was stopped.

To obtain coated cores having a particular T₉₀ value, the appropriate coating solution was uniformly applied to the rotating tablet bed until the desired membrane weight gain was obtained, as described in A, B and C below. At regular intervals throughout the coating process, the weight gain was determined and sample membrane coated units were tested in the release rate assay as described in Example 4 to determine a T₉₀ for the coated units.

The membrane was coated onto the bilayer cores to a weight gain of 40 mg and yielded a dosage form having a T₉₀ of about 6 hours in the release rate assay (i.e., approximately 90% of the drug is released from the dosage form in 6 hours).

The membrane was coated onto the bilayer cores to a weight gain of 59 mg, yielding a dosage form having a T₉₀ of about 8 hours, as determined in the release rate assay.

The membrane was coated onto the bilayer cores to a weight gain of 60 mg and yielded a dosage form having a T₉₀ of about 10 hours in the release rate assay.

Drilling of Membrane Coated Systems

One exit port was drilled into the drug layer end of the membrane coated system.

During the drilling process, samples were checked at regular intervals for orifice size, location, and number of exit ports.

Drying of Drilled Coated Systems

Prior to drying, twinned and broken systems were removed from the batch as necessary. The tablets were manually passed through perforated trays to sort out and remove twinned systems. One exit port was drilled into the coated cores using the LCT laser. The exit port diameter was targeted at 4.5 mm, which was drilled on the drug layer dome of the membrane-coated cores. During the drilling process, three tablets were removed for orifice size measurement periodically. Acceptable Quality Limit (AQL) inspection was performed as well.

Drilled coated systems prepared as above were placed on perforated oven trays and placed on a rack in a relative humidity oven at 45° C. and 45% relative humidity and dried for 72 hours to remove residual solvent. Humidity drying was followed by at least 4 hours of drying at 45° C. and ambient relative humidity.

Application of the Drug Coating

A drug coating was provided over the drilled dosage forms described above. The coating included 6.6 wt % film-forming agent formed of a blend of HPMC 2910 (supplied by Dow) and copovidone (Kollidon® VA 64, supplied by BASF). The HPMC accounted for 3.95 wt % of the drug coating and the Kollidon® VA 64 accounted for 2.65 wt % of the drug coating. The drug coating also included HPC (Klucel® MF) as a viscosity enhancer. The HPC accounted for 1.0 wt% of the drug coating. APAP and HBH were included in the drug coating, with the two drugs accounting for 92.4 wt % of the drug coating. APAP accounted for 90 wt % of the drug coating, HBH accounted for 2.4 wt % of the drug coating.

In order to form the drug coating, an aqueous coating formulation was created using purified water USP as the solvent. The coating formulation included a solids content of 20 wt % and a solvent content of 80 wt %. The solids loaded into the coating formulation were those that formed the finished drug coating, and the solids were loaded in the coating formulation in the same relative proportions as contained in the finished drug coating. Two stainless steel vessels were used initially for mixing two separate polymer solutions, and then the polymer solutions were combined before adding HBH and APAP. Copovidone was dissolved in the first vessel, containing 24 kg of water (⅔ of the total water) followed by the addition of HPMC E-5. This vessel was equipped with two mixers, one of which was set up on the top and the other was located on the side at the bottom of the vessel. The Klucel MF (HPC) was dissolved in the second vessel containing 1200 grams of water (⅓ of the required water). Both polymer solutions were mixed until the solutions were clear. Next, the HPC/water solution was transferred into the vessel, which contained copovidone/HPMC/water. Then, HBH was added and mixed until dissolved completely. Finally, APAP (and optionally Ac-di-sol) was added to the polymer/HBH/water solution. The mixture was stirred continuously until a homogenous suspension was obtained. The suspension was mixed during spraying.

After forming the coating formulation, the drug coating was formed over the drilled dosage forms using a 24-inch High-Coater (CA#66711-1-1) equipped with two Marsterflex peristattic pump heads. All of the three lots were coated to the same target weight gain of 195 mg/core (average coating weight of 199.7 mg).

Color and Clear Overcoats

Optional color or clear coats solutions were prepared in a covered stainless steel vessel. For the color coat, 88 parts of purified water was mixed with 12 parts of Opadry II until the solution was homogeneous. For the clear coat 90 parts of purified water was mixed with 10 parts of Opadry Clear until the solution was homogeneous. The dried cores prepared as above were placed into a rotating, perforated pan coating unit. The coater was started and after the coating temperature was attained (35-45° C.), the color coat solution was uniformly applied to the rotating tablet bed. When a sufficient amount of solution was applied, as conveniently determined when the desired color overcoat weight gain was achieved, the color coat process was stopped. Next, the clear coat solution was uniformly applied to the rotating tablet bed. When a sufficient amount of solution was applied, or the desired clear coat weight gain was achieved, the clear coat process was stopped. A flow agent (e.g., Carnubo wax) can be optionally applied to the tablet bed after clear coat application.

The components which make up the dosage forms described above are set forth as weight percent composition in Table 5 below.

TABLE 6
Formulations for Hydrocodone Bitartrate/Acetaminophen Tablets

| Push Displacement Layer: 138 mg | |
|---|---|
| Polyethylene Oxide, NF, 303, 7000K, TG, LEO | 64.30 |
| Sodium Chloride, USP, Ph Eur, (Powder) | 30.00 |
| Povidone, USP, Ph Eur, (K29-32) | 5.00 |
| Ferric Oxide, NF, (Red) | 0.40 |
| Stearic Acid, NF, Powder | 0.25 |
| BHT, FCC, Ph Eur, (Milled) | 0.05 |
| Drug Layer: 413 mg | |
| Polyethylene Oxide, NF, N-80, 200K, TG, LEO | 2.55 |
| Hydrocodone Bitartrate, USP | 2.42 |
| Acetaminophen, USP (fine powder) | 80.00 |
| Poloxamer F188 (Pluronic F68), NF, Ph Eur | 8.00 |
| Croscarmellose Sodium, NF | 3.00 |
| Povidone, USP, Ph Eur, (K29-32) | 3.00 |
| Stearic Acid, NF, Powder | 0.75 |
| Magnesium Stearate, NF, Ph Eur | 0.25 |
| BHT, FCC, Ph Eur, (Milled) | 0.03 |
| Subcoating: 17 mg | |
| Hydroxyethyl Cellulose, NF | 95.0 |
| Polyethylene Glycol 3350, NF, LEO | 5.0 |
| Membrane Coating*: 40 mg, 59 mg, 60 mg (for a | |
| T₉₀ of 6 hrs, 8 hrs, and 10 hrs, respectively) | |
| Cellulose Acetate, NF, (398-10) | 75.0 (80.0) |
| Poloxamer F188 (Pluronic F68), NF, Ph Eur | 25.0 (20.0) |
| BHT, FCC, Ph Eur, (Milled) | Trace (0.0003) |
| Drug Coating: 195 mg | |
| Hydrocodone Bitartrate, USP | 2.40 |
| Acetaminophen, USP (fine powder) | 90.00 |
| HPMC 2910, USP, Ph Eur, 5 cps | 3.96 |
| Copovidone, Ph Eur, JPE | 2.64 |
| Hydroxypropyl Cellulose, NF, MF | 1.00 |
| Color Overcoat: 30 mg | |
| OPADRY, White (YS-2-7063) | 100.00 |

75/25 CA398-10/Pluronic F68 used for the 6 h and 8 hr systems
*80/20 CA398-10* 80/20 CA398-10/Pluronic F68 used for the 10 h system.

Dosage forms manufactured as described above were tested in release rate assays as described in Example 4, and were tested in humans in a clinical trial described in Example 5 below.

### EXAMPLE 2

An alternative formulation was prepared according to the procedures described in Example 1 above, varying certain of the constituents.

The components which make up the dosage forms are set forth as weight 5 percent composition in Table 7 below.

TABLE 7
Formulations for Hydrocodone Bitartrate/Acetaminophen Tablets

| Push Displacement Layer: 138 mg | |
|---|---|
| Polyethylene Oxide, NF, 303, 7000K, TG, LEO | 64.30 |
| Sodium Chloride, USP, Ph Eur, (Powder) | 30.00 |
| Povidone, USP, Ph Eur, (K29-32) | 5.00 |
| Ferric Oxide, NF, (Red) | 0.40 |
| Stearic Acid, NF, Powder | 0.25 |
| BHT, FCC, Ph Eur, (Milled) | 0.05 |
| Drug Layer: 413 mg | |
| Polyethylene Oxide, NF, N-80, 200K, TG, LEO | 2.55 |
| Hydrocodone Bitartrate, USP | 2.42 |
| Acetaminophen, USP (fine powder) | 80.00 |
| Poloxamer F188 (Pluronic F68), NF, Ph Eur | 8.00 |
| Croscarmellose Sodium, NF | 3.00 |
| Povidone, USP, Ph Eur, (K29-32) | 3.00 |
| Stearic Acid, NF, Powder | 0.75 |
| Magnesium Stearate, NF, Ph Eur | 0.25 |
| BHT, FCC, Ph Eur, (Milled) | 0.03 |
| Subcoating: 10 mg | |
| Hydroxyethyl Cellulose, NF | 95.0 |
| Polyethylene Glycol 3350, NF, LEO | 5.0 |
| Membrane Coating*: 63 mg (for a T₉₀ of 8 hrs) | |
| Cellulose Acetate, NF, (398-10) | 77.0 |
| Poloxamer F188 (Pluronic F68), NF, Ph Eur | 23.0 |
| BHT, FCC, Ph Eur, (Milled) | Trace (0.0003) |
| Drug Coating: 195 mg | |
| Hydrocodone Bitartrate, USP | 2.76 |
| Acetaminophen, USP (fine powder) | 87.40 |
| HPMC 2910, USP, Ph Eur, 5 cps | 3.50 |
| Copovidone, Ph Eur, JPE | 2.34 |
| Hydroxypropyl Cellulose, NF, MF | 1.00 |
| Croscarmellose Sodium, NF | 3.00 |
| Color Overcoat: 15 mg | |
| OPADRY, White (YS-2-7063) | 100.00 |

77/23 CA398-10/Pluronic F68
*80/20 CA398-10

The dosage forms were prepared using the procedures described in Example 1, and contained the composition set forth above. The dosage forms were tested in release rate assays as described in Example 4, and tested in humans in a clinical trial described in Example 6 below.

### EXAMPLE 3

Additional formulations were prepared according to the procedures described in Example 1 above, varying the amounts of the binder. In particular, four formulations having identical compositions to the formulation of Example 1 were prepared, with the following exceptions:

The drug layer composition was prepared as described, using a finer grade of acetaminophen (Ph Eur Fine Powder), utilizing a push displacement layer containing a lower amount of polyethylene oxide, NF, 303, 7000K, TG, LEO (61.3%), and an additional 3% glyceryl behenate, NF, Ph Eur, using a different grade of hydroxyethylcellulose in the subcoat (NF, Ph Eur, 250 LPH), and a drug coating containing a different amount and grade of acetaminophen (87.584%, Ph Eur micronized) and a lower amount of hydrocodone bitartrate (2.576%);

The drug layer composition was prepared as described, using 2.55% hydroxypropylcellulose EXF instead of polyethylene oxide N-80, a lower amount of acetaminophen (78.787%) and a finer grade (Ph Eur (Fine Powder), a lower amount of hydrocodone bitartrate (2.383%), 1.375% stearic acid, NF, 0.5% colloidal silicon dioxide, NF, and 0.375% magnesium stearate, and utilizing a push displacement layer containing 61.3% polyethylene oxide, NF, 303, 7000K, TG, LEO, and including an additional 3% hydropropylcellulose, and a drug coating containing a different amount and grade of acetaminophen (87.584%, Ph Eur micronized) and a lower amount of hydrocodone bitartrate (2.576%); and

The drug layer composition was prepared as described, using 4.55% hydroxypropylcellulose EXF as a substitute for polyox N-80, a lower amount of acetaminophen (76.845% Fine Powder), 2.325% hydrocodone bitartrate, 1.375% stearic acid, NF, 0.5% colloidal silicon dioxide, NF, and 0.375% magnesium stearate, and utilizing a push displacement layer containing 61.3% polyethylene oxide, NF, 303, 7000K, TG, LEO, and an additional 3% hydropropylcellulose, and a drug coating containing a different amount and grade of acetaminophen (87.584%, Ph Eur micronized) and a lower amount of hydrocodone bitartrate (2.576%).

The drug layer composition was prepared as described, using 2.55% hydroxypropylcellulose EXF as a substitute for polyox N-80, acetaminophen (78.56% Fine Powder), 2.38% hydrocodone bitartrate, 1.5% stearic acid, NF, 0.5% colloidal silicon dioxide, NF, 0.5% magnesium stearate, and 0.01% BHT, and utilizing a push displacement layer containing 61.3% polyethylene oxide, NF, 303, 7000K, TG, LEO, and an additional 3% hydropropylcellulose, and a drug coating containing acetaminophen (90.0%, Ph Eur micronized), hydrocodone bitartrate (2.56%), Copovidone (2.56%), HPMC (3.88%) and HPC (1.0%). The total weight of the drug coating was 194 mg, the weight of the drug layer was 420 mg and the push layer weight was 140 mg.

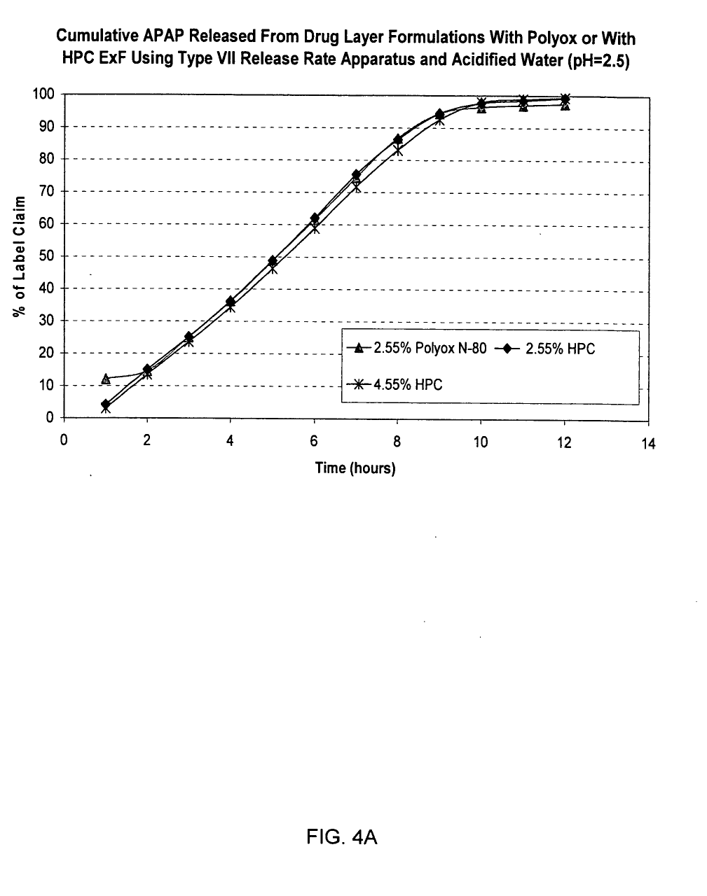

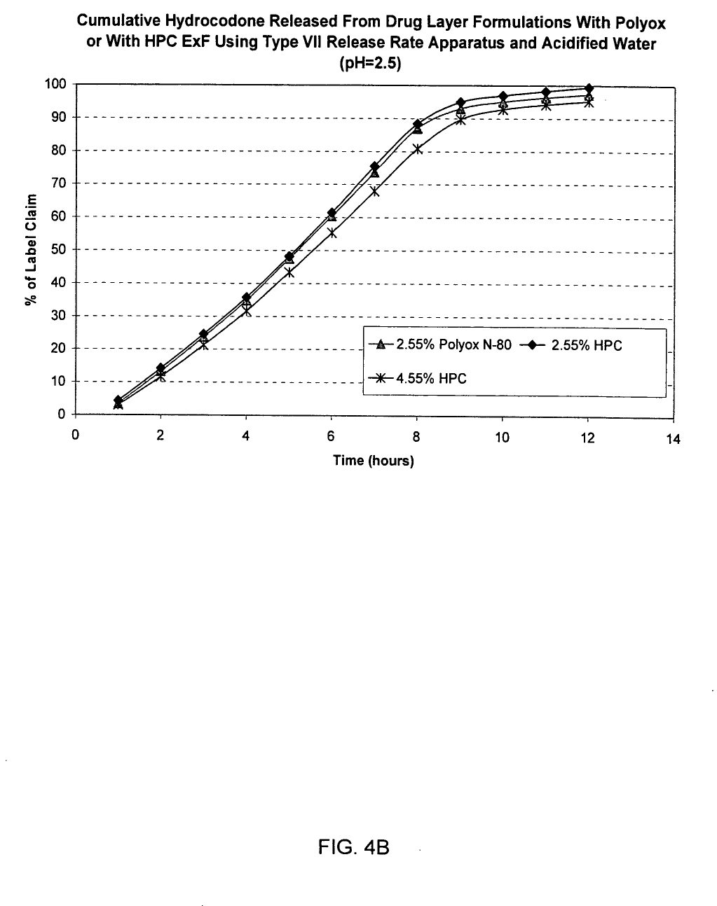

The release rates of acetaminophen and hydrocodone from the first three of these additional dosage forms are shown in FIGS. 4A and 4B. The graphs show that the dosage forms provide similar release profiles of acetaminophen and hydrocodone. The graphs also show that the two drugs were released at relatively proportional rates with substantially complete delivery of the active agents.

### EXAMPLE 4

The release rate of drug from the dosage forms described above was determined in the following standardized assay. The method involves releasing systems into 900 ml acidified water (pH 3). Aliquots of sample release rate solutions were injected onto a chromatographic system to quantify the amount of drug released during specified test intervals. Drugs were resolved on a C₁₈ column and detected by UV absorption (254 nm for acetaminophen). Quantitation was performed by linear regression analysis of peak areas from a standard curve containing at least five standard points.

Samples were prepared with the use of a USP Type 7 Interval Release Apparatus. Each dosage form to be tested was weighed, then glued to a plastic rod having a sharpened end, and each rod was attached to a release rate dipper arm. Each release rate dipper arm was affixed to an up/down reciprocating shaker (USP Type 7 Interval Release Apparatus), operating at an amplitude of about 3 cm and 2 to 4 seconds per cycle. The rod ends with the attached systems were continually immersed in 50 ml calibrated test tubes containing 50 ml of acidified H₂O (acidified to pH 3.00.±.0.05 with phosphoric acid), equilibrated in a constant temperature water bath controlled at 37° C.±0.5° C. At the end of each time interval of 90 minutes, the dosage forms were transferred to the next row of test tubes containing fresh acidified water. The process was repeated for the desired number of intervals until release was complete. Then the solution tubes containing released drug were removed and allowed to cool to room temperature. After cooling, each tube was filled to the 50 ml mark with acidified water, each of the solutions was mixed thoroughly, and then transferred to sample vials for analysis by high pressure liquid chromatography (HPLC). Standard solutions of drug were prepared in concentration increments encompassing the range of 5 micrograms to about 400 micrograms and analyzed by HPLC. A standard concentration curve was constructed using linear regression analysis. Samples of drug obtained from the release test were analyzed by HPLC and concentrations of drug were determined by linear regression analysis. The amount of drug released in each release interval was calculated.

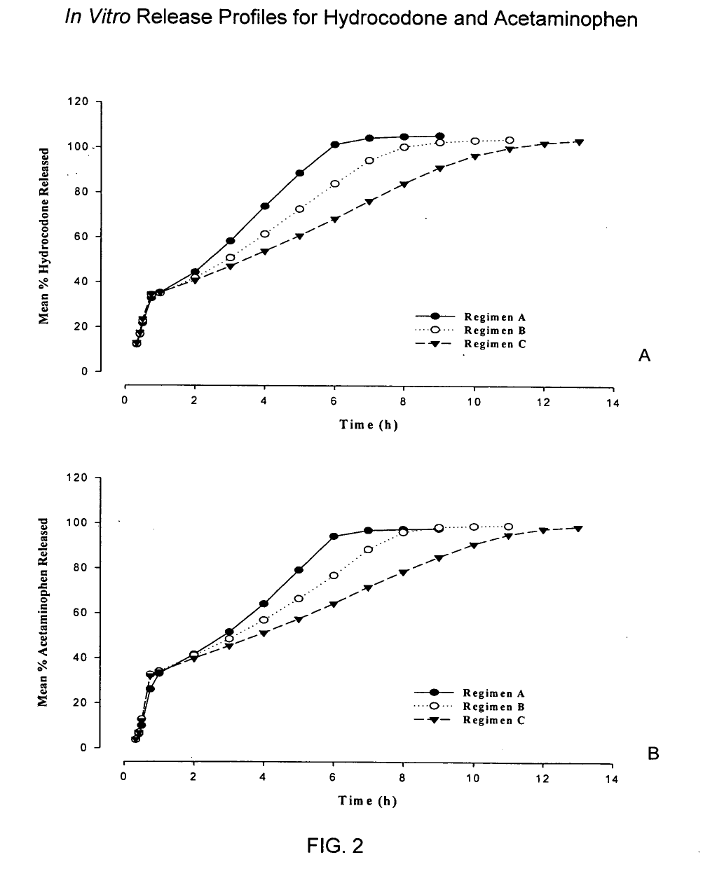

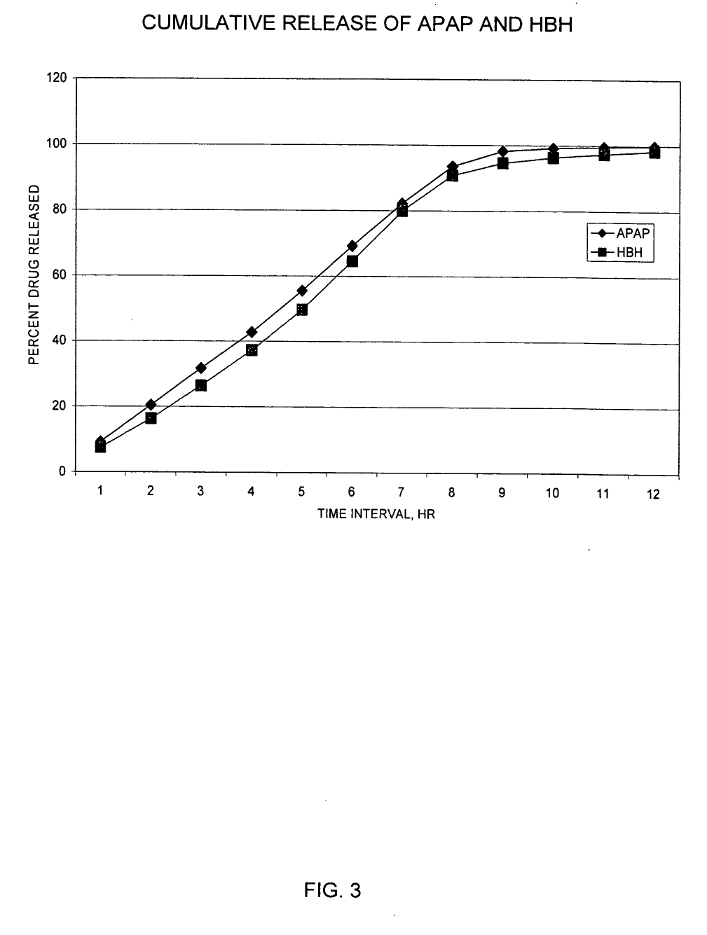

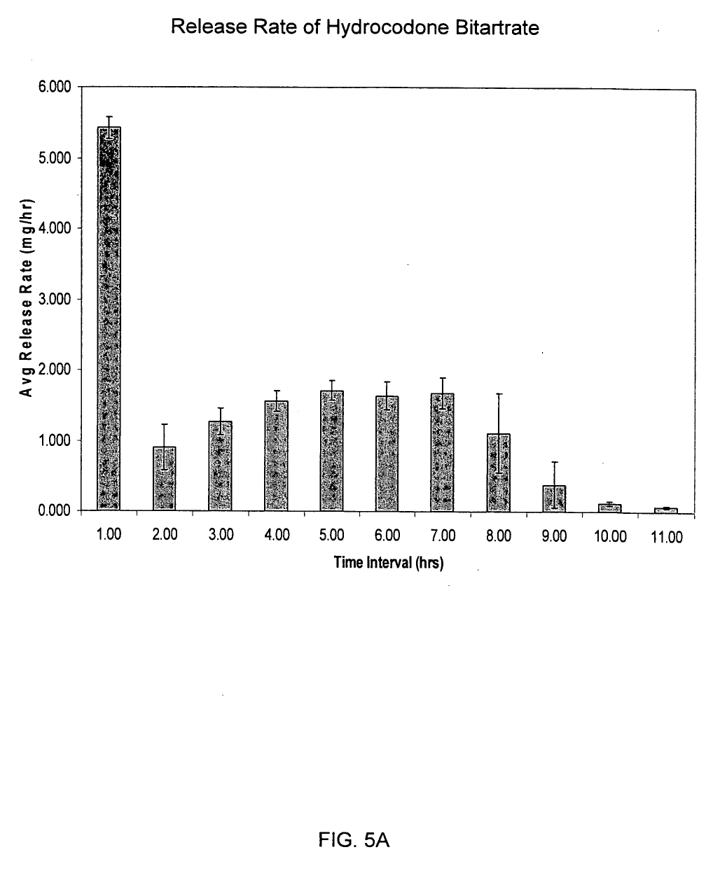

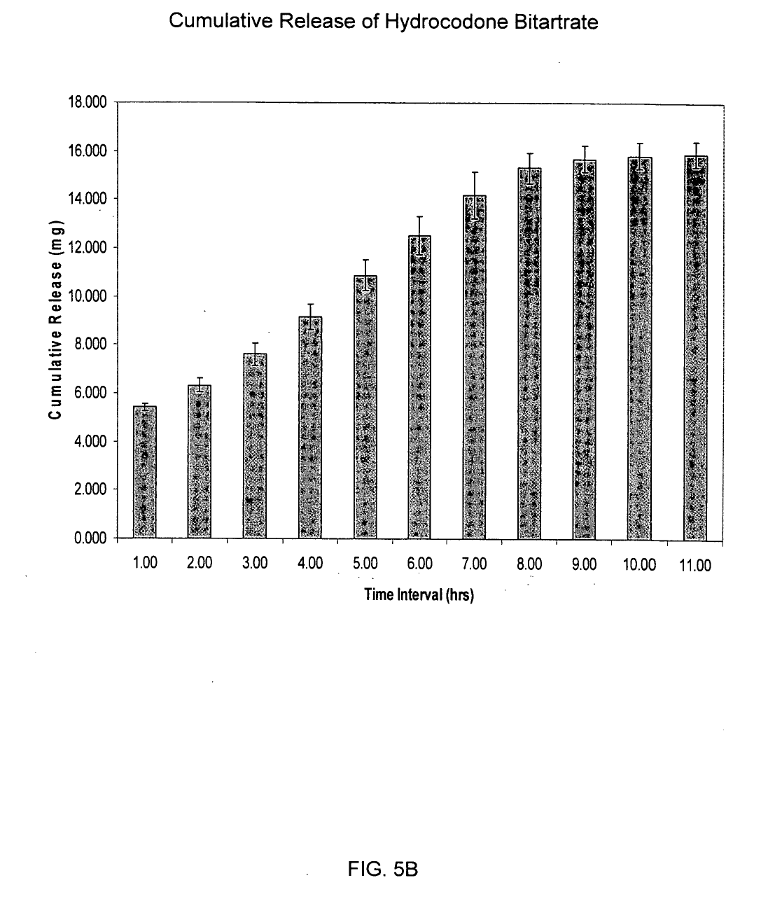

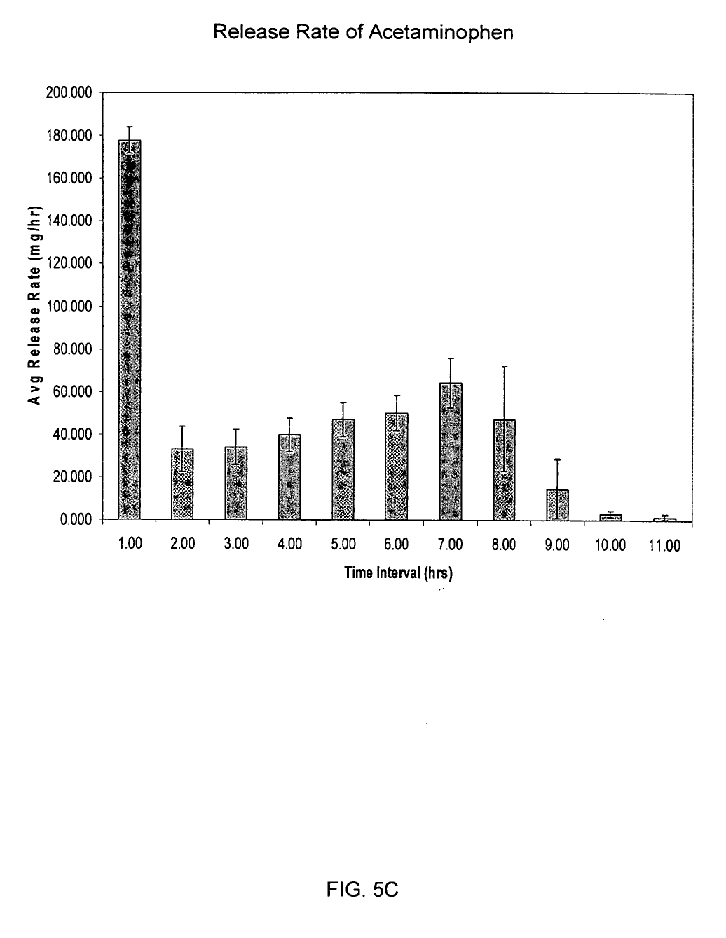

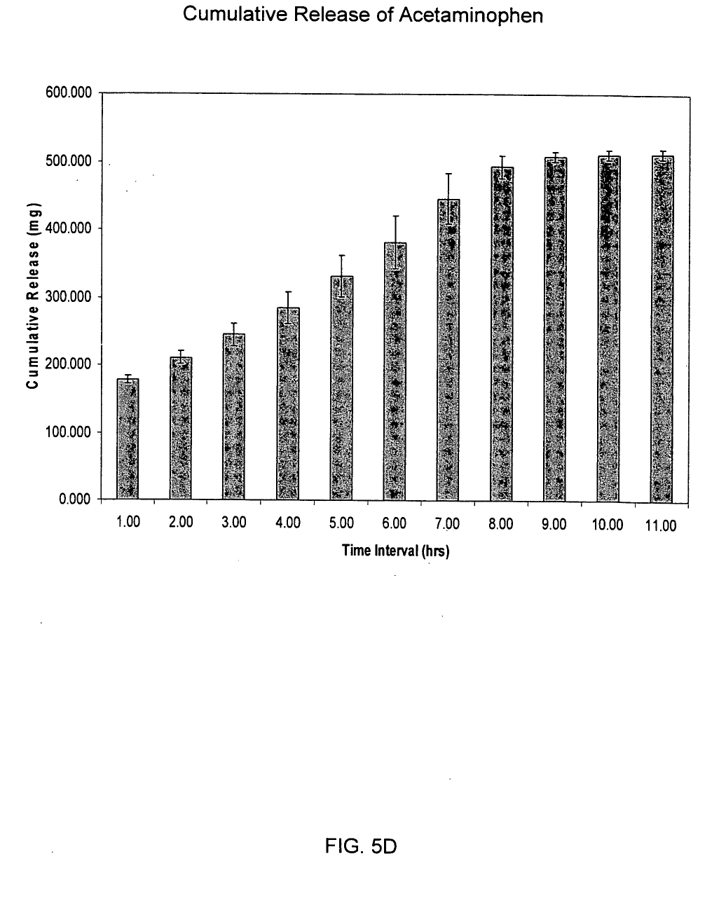

The release rate assay results for various dosage forms of the invention are illustrated in FIGS. 2-7. Dosage forms having a membrane coating weight of 59 mg of 75/25 CA398-10/Pluronic F68 were shown to exhibit a T₉₀ of about 8 hours, as shown in FIGS. 2A and 2B, the cumulative release rate graph illustrated in FIG. 3 and FIGS. 5A-D. As can be seen from FIGS. 2 and 3, dosage forms release acetaminophen and hydrocodone at an ascending rate of release, whereby the percent drug released as a function of time does not exhibit a constant rate of release, but instead increases slightly with time until about 80% to 90% of the drug is released. The increase in the rate of release of acetaminophen and hydrocodone is due to the increased osmotic activity of the push displacement layer as the drug layer is expelled, and was observed in the absence as well as the presence of the drug coating. As shown in FIGS. 2A and 2B and FIG. 5A, dosage forms having a drug coating also exhibit an ascending rate of release, and exhibit an initial release of about ⅓ of the total dose from the drug coating. An initial peak hydrocodone release rate was observed occurring within one hour, and a second peak release rate was observed occurring within about 5 to 7 hours after introduction of the dosage form into the aqueous environment of the release assay. FIG. 5C also demonstrates the initial release of acetaminophen from the drug coating, followed by a slightly ascending rate of release until about 7 hours. The cumulative drug released is shown in FIGS. 5B and 5D, for hydrocodone and acetaminophen, respectively, and demonstrates the initial drug release, followed by a slightly ascending rate of release.

Dosage forms having a membrane coating weight of 40 mg of 75/25 CA398-10/Pluronic F68 were shown to exhibit a T₉₀ of about 6 hours, as shown in FIGS. 2A and 2B and FIGS. 6A-D. As shown in FIG. 6A, dosage forms having a drug coating exhibit an initial release of about ⅓ of the total dose of hydrocodone from the drug coating, followed by an ascending rate of release of hydrocodone to a second peak release rate occurring within about 4 to 6 hours. FIG. 6C demonstrates the initial release of acetaminophen from the drug coating, followed by a slightly ascending rate of release for about 5-6 hours. The cumulative drug released is shown in FIGS. 6B and 6D, for hydrocodone and acetaminophen, respectively, and demonstrates the initial drug release, followed by a slightly ascending rate of release.

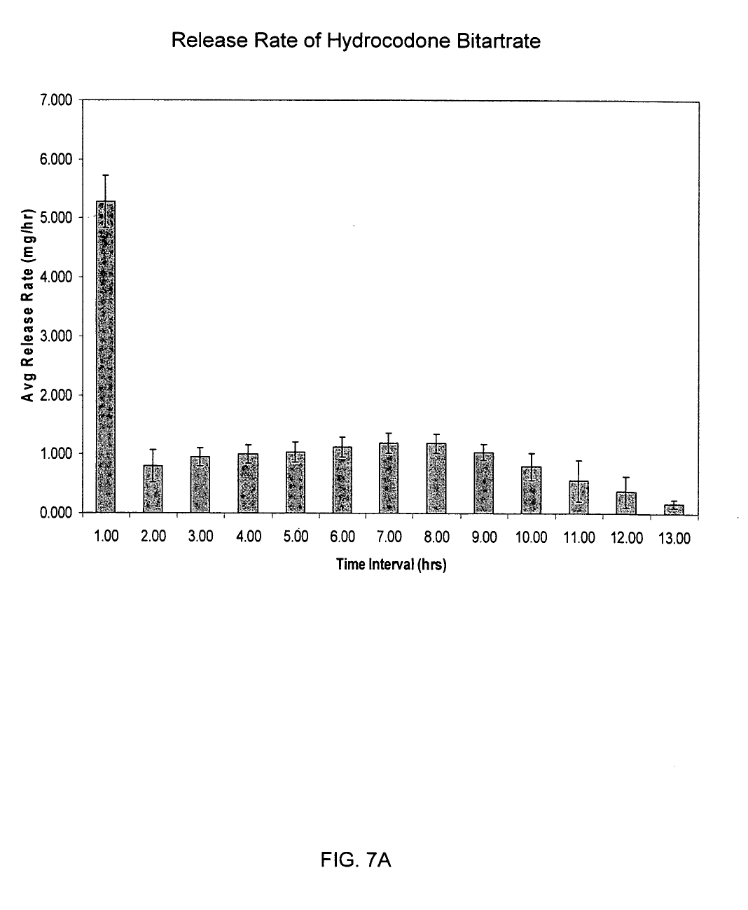

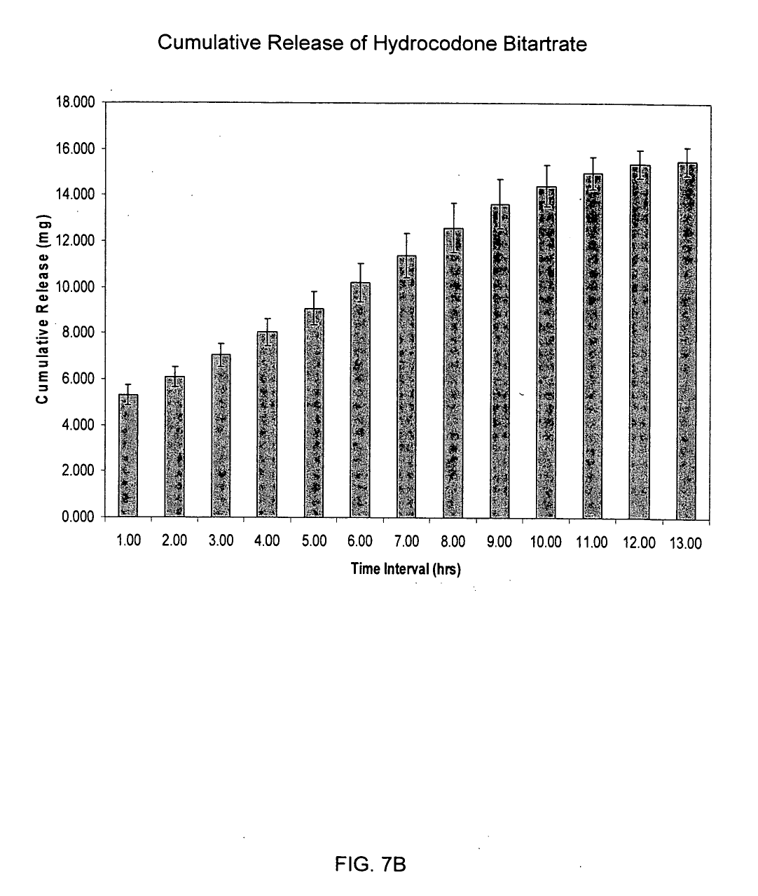

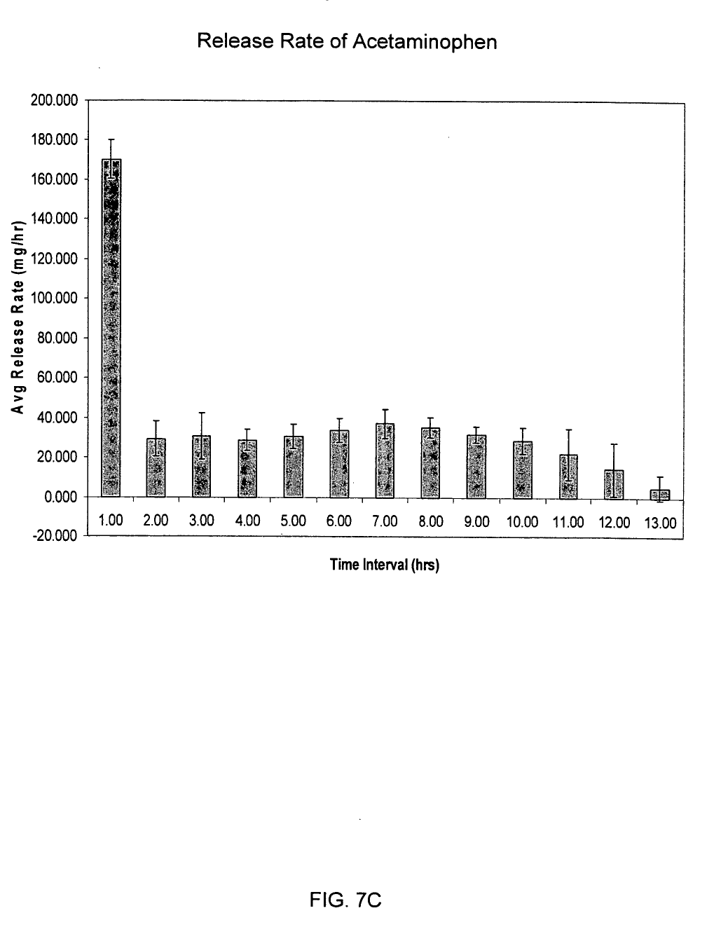

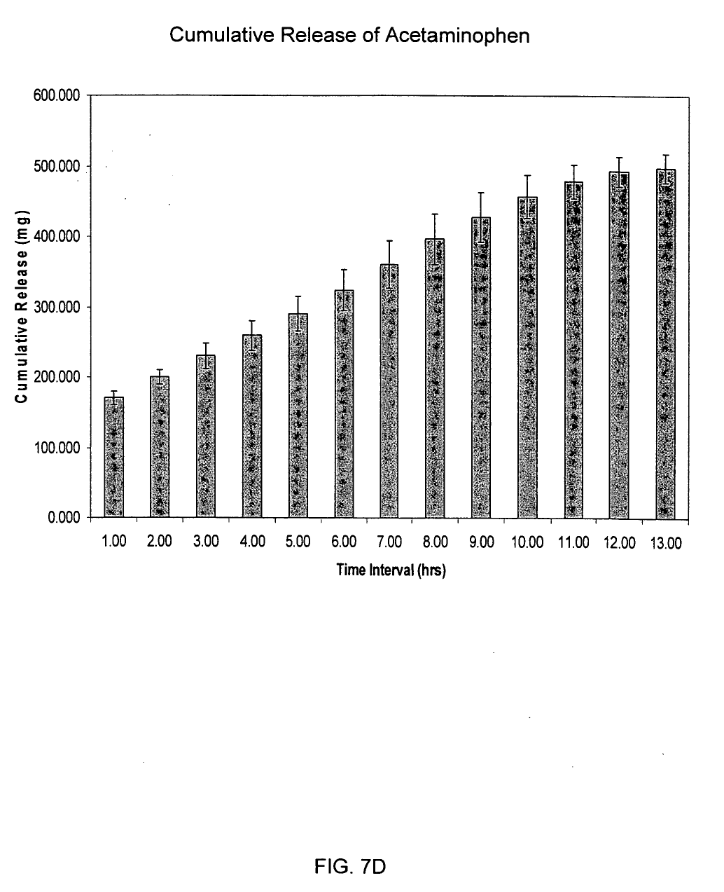

Dosage forms having a membrane coating weight of 60 mg of 80/20 CA398-10/Pluronic F68 were shown to exhibit a T₉₀ of about 10 hours, as shown in FIGS. 2A and 2B and FIGS. 7A-D. These dosage forms demonstrate a flatter release profile, and more closely resemble a zero order rate of release than the preceding systems characterized by having T₉₀ values of 6 and 8 hours. As shown in FIG. 7A, dosage forms having a drug coating exhibit an initial release of about ⅓ of the total dose of hydrocodone from the drug coating, followed by a slightly ascending rate of release of hydrocodone to a second peak release rate occurring within about 7 to 8 hours. FIG. 7C demonstrates the initial release of acetaminophen from the drug coating, followed by a slightly ascending rate of release for about 5-6 hours. The cumulative drug released is shown in FIGS. 7B and 7D, for hydrocodone and acetaminophen, respectively, and demonstrates the initial drug release, followed by a slightly ascending rate of release.

The results of the release rate assays performed on samples A, B and C from Example 1 are set forth in Tables 8 and 9 below.

TABLE 8
Release pattern for acetaminophen (% released)

| | | | |
|---|---|---|---|
| Time | 6 hour | 8 hour | 10 hour |
| interval | formulation | formulation | formulation |

| | | | | |
|---|---|---|---|---|
| 0-20 | min | 4 | 4 | 4 |
| 0-25 | min | 6 | 7 | 7 |
| 0-30 | min | 10 | 13 | 12 |
| 0-45 | min | 26 | 34 | 32 |
| 0-1 | hour | 33 | 36 | 34 |
| 0-2 | hours | 42 | 42 | 40 |
| 0-3 | hours | 52 | 49 | 46 |
| 0-4 | hours | 64 | 57 | 51 |
| 0-5 | hours | 79 | 66 | 58 |
| 0-6 | hours | 94 | 76 | 64 |
| 0-7 | hours | 97 | 89 | 72 |
| 0-8 | hours | 98 | 99 | 79 |
| 0-9 | hours | 98 | 102 | 85 |
| 0-10 | hours | | 102 | 91 |
| 0-11 | hours | | 102 | 95 |
| 0-12 | hours | | | 98 |
| 0-13 | hours | | | 99 |

| | | | |
|---|---|---|---|
| residual | 0 | 1 | 1 |

TABLE 9
Release pattern for hydrocodone (% released)

| Time | | 6 hour | 8 hour | 10 hour |
|---|---|---|---|---|
| interval | | formulation | formulation | formulation |
| 0-20 | min | 12 | 13 | 13 |
| 0-25 | min | 17 | 18 | 18 |
| 0-30 | min | 22 | 24 | 24 |
| 0-45 | min | 33 | 35 | 35 |
| 0-1 | hour | 35 | 36 | 35 |
| 0-2 | hours | 44 | 42 | 41 |
| 0-3 | hours | 58 | 51 | 47 |
| 0-4 | hours | 74 | 61 | 54 |
| 0-5 | hours | 89 | 73 | 61 |
| 0-6 | hours | 101 | 83 | 68 |
| 0-7 | hours | 104 | 95 | 76 |
| 0-8 | hours | 105 | 102 | 84 |
| 0-9 | hours | 105 | 105 | 91 |
| 0-10 | hours | | 105 | 97 |
| 0-11 | hours | | 106 | 100 |
| 0-12 | hours | | | 102 |
| 0-13 | hours | | | 103 |

| | | | |
|---|---|---|---|
| residual | 0 | 1 | 3 |

### EXAMPLE 5

The in vivo efficacy and safety of the dosage forms prepared in Example 1 were tested as follows:

Twenty-four healthy volunteers, twelve male and twelve female, were enrolled in a Phase I clinical trial of open label randomized four period crossover study design. An equal number of male subjects and female subjects were paired together in one of four groups. Subjects within each gender category were randomly assigned to the four sequences of regimens described below to avoid sequence bias and confounding of sequence and gender.

Four treatment options were tested in sequence, with a single treatment regimen administered on Study Day 1. A wash out period of at least 6 days was included to separate the dosing days. Each treatment group received each of the four treatments during the course of the study, as shown in Table 10 below with one exception. That exception was not included in the analysis of pharmacokinetic parameters. For the each of the four periods, subjects were given one of the four treatment options by oral administration, as follows:
a controlled release HBH/APAP product prepared by the method described in Example 1 (two tablets totaling 30 mg HBH and 1000 mg APAP), having a target T₉₀ value of approximately 6 hours (Regimen A);
a controlled release HBH/APAP product prepared by the method described in Example 1 (two tablets totaling 30 mg HBH and 1000 mg APAP), having a target T₉₀ value of approximately 8 hours (Regimen B);
a controlled release HBH/APAP product prepared by the method described in Example 1 (two tablets totaling 30 mg HBH and 1000 mg APAP), having a target T₉₀ value of approximately 10 hours (Regimen C); or

The reference drug NORCO®, an immediate release formulation of HBH and APAP containing 10 mg HBH and 325 mg APAP, administered every four hours for a total of three administrations over a 12 hour period (Regimen D).

TABLE 10
Regimen Sequence

| Sequence | Number of | | | | |
|---|---|---|---|---|---|
| Group | Subjects | Period 1 | Period 2 | Period 3 | Period 4 |
| I | M = 3, F = 3 | Regimen A | Regimen B | Regimen C | Regimen D |
| II | M = 3, F = 3 | Regimen B | Regimen D | Regimen A | Regimen C |
| III | M = 3, F = 3 | Regimen C | Regimen A | Regimen D | Regimen B |
| IV | M = 3, F = 3 | Regimen D | Regimen C | Regimen B | Regimen A |

The controlled release product of Regimens A-C and the first dose of Regimen D were administered on Study Day 1 under stringent fasting conditions. Blood samples were collected from each subject receiving treatment Regimens A-C for pharmacokinetic sampling at approximate times after administration as follows: 0, 0.25 hr, 0.5 hr, 0.75 hr, 1 hr, 2 hr, 3 hr, 4 hr, 6 hr, 8 hr, 10 hr, 12 hr, 16 hr, 20hr, 24 hr, 36 hr, 48 hr. For subjects receiving treatment Regimen D, blood samples were collected at approximate times after administration of the first dose as follows: 0, 0.25 hr, 0.5 hr, 0.75 hr, 1 hr, 2 hr, 4 hr, 4.25 hr, 4.5 hr, 5 hr, 6 hr, 8 hr, 8.25 hr, 8.5 hr, 9 hr, 10 hr, 12 hr, 16 hr, 20 hr, 24 hr, 36 hr, 48 hr.

Blood samples were processed to separate plasma for further analysis, and plasma concentrations of hydrocodone and acetaminophen were determined using a validated HPLC/MS/MS method with quantitation between 0.092 and 92 ng/mL for hydrocodone and 5 and 10,000 ng/mL for acetaminophen.

Values for the pharmacokinetic parameters of hydrocodone and acetaminophen were estimated using noncompartmental methods. Plasma concentrations were adjusted for potency in the determination of pharmacokinetic parameters.

The maximum observed plasma concentration (Cₘₐₓ) and the time to Cₘₐₓ (peak time, Tₘₐₓ) were determined directly from the plasma concentration-time data. The value of the terminal phase elimination rate constant (β) was obtained from the slope of the least squares linear regression of the logarithms of the plasma concentration versus time data from the terminal log-linear phase of the profile. The terminal log-linear phase was identified using WinNonlin-Professional™, Version 4.0.1 (Pharsight Corporation, Mountain View, Calif.) and visual inspection. A minimum of three concentration-time data points was used to determine β. The terminal phase elimination half-life (t_{1/2}) was calculated as ln(2)/β.

The area under the plasma concentration-time curve (AUC) from time 0 to the time of the last measurable concentration (AUCₜ) was calculated by the linear trapezoidal rule. The AUC was extrapolated to infinite time by dividing the last measurable plasma concentration (Cₜ) by β. Denoting the extrapolated portion of the AUC by AUCₑₓₜ, the AUC from time 0 to infinite time (AUC_{∞}) was calculated as follows:
AUC_{∞}=AUCₜ+AUCₑₓₜ

The percentage of the contribution of the extrapolated AUC (AUCₑₓₜ) to the overall AUC_{∞} was calculated by dividing the AUCₑₓₜ by the AUC_{∞} and multiplying this quotient by 100. The apparent oral clearance value (CL/F, where F is the bioavailability) was calculated by dividing the administered dose by the AUC_{∞}.

Plasma concentrations of hydrocodone and acetaminophen along with their pharmacokinetic parameter values were tabulated for each subject and each regimen, and summary statistics were computed for each sampling time and each parameter.

The bioavailability of each CR regimen relative to that of the IR regimen was assessed by a two one-sided tests procedure via 90% confidence intervals obtained from the analyses of the natural logarithms of AUC. These confidence intervals were obtained by exponentiating the endpoints of confidence intervals for the difference of mean logarithms

The above analysis was performed on pharmacokinetic parameters adjusted for potency

Results

The plasma concentrations of hydrocodone and acetaminophen are shown in Tables 2-5 discussed above and FIGS. 8A and 8B. As these figures illustrate, volunteers receiving two tablets of each of the three dosage forms prepared according the procedure of Example 1 exhibited a rapid rise in plasma concentrations of hydrocodone and acetaminophen after oral administration at time zero. The plasma concentrations of hydrocodone and acetaminophen reach an initial peak due to the release of hydrocodone and acetaminophen from the drug coating. Subsequent to the initial release of hydrocodone and acetaminophen, the sustained release of the dosage forms provides for continued release of hydrocodone and acetaminophen to the patient.

The test Regimens A (6 hour release prototype), B (8 hour release prototype) and C (10 hour release prototype) were equivalent to the reference Regimen D (NORCO®) with respect to AUC for both hydrocodone and acetaminophen because the 90% confidence intervals for evaluating bioequivalence were contained within the 0.80 to 1.25 range.

Test Regimen A was equivalent to the reference Regimen D with respect to hydrocodone Cₘₐₓ because the 90% confidence interval for evaluating bioequivalence was contained within the 0.80 to 1.25 range. Compared to Regimen D, hydrocodone Cₘₐₓ central values for Regimens B and C were 16% and 25% lower. Compared to Regimen D, acetaminophen Cₘₐₓ central values for Regimens A, B and C were 9% to 13% lower.

### EXAMPLE 6

The in vivo efficacy and safety of additional dosage forms prepared as described in Example 2 were tested in a second clinical trial. The study protocol and results are described below.

Methods:

Forty-four healthy volunteers, twenty two male and twelve female, were enrolled in a Phase I two-part, single-dose and multiple-dose, fasting and nonfasting, study of open label, randomized, dose-proportionality and steady state study design. Two male subjects and two female subjects were paired together in one of six sequence groups in Cohort I in a crossover design for a total of 24 subjects. Five male subjects and five female subjects were paired together in one of two groups in Cohort II for a total of 20 subjects. Subjects within each gender category were randomly assigned to the six sequences of regimens within Cohorts I and II described below to avoid sequence bias and confounding of sequence and gender.

For Cohort I, four treatment options were tested in sequence, with a single treatment regimen administered on Study Day 1. A wash out period of at least 5 days was included. Each treatment group received each of the four treatments during the course of the study, as shown in Table 11 below. For the each of the four periods, subjects were given one of the four treatment options by oral administration, as follows:
a controlled release HBH/APAP product prepared by the method described in Example 2 (one tablet totaling 15 mg HBH and 500 mg APAP), having a T₉₀ value of 8 hours (Regimen A);
a controlled release HBH/APAP product prepared by the method described in Example 2 (two tablets totaling 30 mg HBH and 1000 mg APAP), having a T₉₀ value of 8 hours (Regimen B);
a controlled release HBH/APAP product prepared by the method described in Example 2 (three tablets totaling 45 mg HBH and 1500 mg APAP), having a T₉₀ value of 8 hours (Regimen C); or

The reference drug NORCO®, one immediate release formulation of HBH and APAP containing 10 mg HBH and 325 mg APAP, administered every four hours for a total of three administrations over a 12 hour period (Regimen D).

TABLE 11
Regimen Sequence

| Sequence | Number of | | | | |
|---|---|---|---|---|---|
| Group | Subjects | Period 1 | Period 2 | Period 3 | Period 4 |
| I | M = 2, F = 2 | Regimen A | Regimen B | Regimen D | Regimen C |
| II | M = 2, F = 2 | Regimen A | Regimen D | Regimen B | Regimen C |
| III | M = 2, F = 2 | Regimen B | Regimen A | Regimen D | Regimen C |
| IV | M = 2, F = 2 | Regimen B | Regimen C | Regimen A | Regimen C |
| V | M = 2, F = 2 | Regimen D | Regimen D | Regimen B | Regimen C |
| VI | M = 2, F = 2 | Regimen D | Regimen B | Regimen A | Regimen C |

The controlled release product of Regimens A-C and the first dose of Regimen D was administered on Study Day 1 under stringent fasting conditions. Blood samples were collected from each subject receiving treatment Regimens A-C for pharmacokinetic sampling at approximate times after administration as follows: 0, 0.25 hr, 0.5 hr, 0.75 hr, 1 hr, 2 hr, 3 hr, 4 hr, 6 hr, 8 hr, 10 hr, 12 hr, 16 hr, 20 hr, 24 hr, 36 hr, 48 hr. For subjects receiving treatment Regimen D, blood samples were collected at approximate times after administration of the first dose as follows: 0, 0.25 hr, 0.5 hr, 0.75 hr, 1 hr, 2 hr, 4 hr, 4.25 hr, 4.5 hr, 5 hr, 6 hr, 8 hr, 8.25 hr, 8.5 hr, 9 hr, 10 hr, 12 hr, 16 hr, 20 hr, 24 hr, 36 hr, 48 hr. Blood samples were processed to separate plasma for further analysis, and plasma concentrations of hydrocodone, acetaminophen and hydromorphone were determined. Blood samples were also tested for pharmacogenetic analysis to identify poor and nonpoor metabolizers (CYP2D6 genotypes). Analytical procedures were performed using a validated HPLC/MS/MS with quantitation between 0.092 and 92 ng/mL for hydrocodone, 5 and 10,000 ng/mL for acetaminophen and 0.1 and 100 ng/mL hydromorphone. One subject was excluded from the analysis of pharmacokinetic parameters. CYP2D6 Poor metabolizers were excluded from the analysis of hydromorphone pharmacokinetic parameters.

For Cohort II, two treatment options were tested in sequence, with a single treatment regimen administered on Study Day 1. A wash out period of at least 5 days was included to separate the dosing days of the two study periods. Each treatment group received each of the two treatments during the course of the study, as shown in Table 12 below with the exception of two individuals in group VIII who dropped out during the first period. For the each of the two periods, subjects were given one of the two treatment options by oral administration, as follows:

A controlled release HBH/APAP product prepared by the method described in Example 2 (two tablets totaling 30 mg HBH and 1000 mg APAP), having a T₉₀ value of 8 hours, administered twice a day for 3 consecutive days for a total of 6 doses (180 mg hydrocodone and 6000 mg acetaminophen, Regimen E); or the reference drug NORCO®, one immediate release formulation of HBH and APAP containing 10 mg HBH and 325 mg APAP, administered every four hours for 3 consecutive days for a total of 18 doses (180 mg hydrocodone and 5850 mg acetaminophen, Regimen F).

TABLE 12
Regimen sequence

| | Number of | | |
|---|---|---|---|
| Sequence Group | Subjects | Period 1 | Period 2 |
| VII | 10 | Regimen E | Regimen F |
| VIII | 10 | Regimen F | Regimen E |

The controlled release product of Regimen E and the first dose of Regimen F were administered on Study Day 1 under stringent fasting conditions. Blood samples were collected from each subject receiving treatment Regimen E for pharmacokinetic sampling at approximate times after administration of the first dose as follows: 24 hr (pre-dose 3); 36 hr (pre-dose 4), 48 hr (pre-dose 5), 48.5 hr, 49 hr, 50 hr, 52 hr, 54 hr, 56 hr, 58 hr, 60 hr (pre-dose 6), 60.5 hr, 61 hr, 62 hr, 64 hr, 68 hr, 72 hr, 84 hr, and 96 hr. Blood samples were collected from each subject receiving treatment Regimen F for pharmacokinetic sampling at approximate times after administration as follows: 24 hr (pre-dose 7); 36 hr (pre-dose 10), 48 hr (pre-dose 13), 48.5 hr, 49 hr, 50 hr, 52 hr, 52.25 hr, 52.5 hr, 53 hr, 54 hr, 56 hr, 56.25 hr, 56.5 hr, 57 hr, 58 hr, 60 hr (pre-dose 16), 60.5 hr, 61 hr, 62 hr, 64 hr, 68 hr, 72 hr, 84 hr, and 96 hr. CYP2D6 Poor metabolizers were excluded from the analysis of hydromorphone pharmacokinetic parameters.

The dose of two 15 mg hydrocodone/500 mg tablets administered twice a day is designed to release 10 mg hydrocodone and 340 mg acetaminophen contained in the drug coating and the core is designed to release another 20 mg hydrocodone and 660 mg acetaminophen over an extended period of time. One and three tablet doses were also studied to assess dose proportionality.

The pharmacokinetic analyses of plasma concentrations of hydrocodone and acetaminophen described in Example 5 were performed as described with the exception that potency correction was not performed.

Results

The plasma concentrations of hydrocodone and acetaminophen are shown in Tables 2-4 discussed above and FIGS. 9, 10, and 12-17. As FIGS. 9 and 10 illustrate, volunteers receiving one to three tablets of the dosage form having a T₉₀ of 8 hours prepared according the procedure of Example 2 exhibited a rapid rise in plasma concentrations of hydrocodone and acetaminophen after oral administration at time zero. The plasma concentrations of hydrocodone and acetaminophen reach an initial peak due to the release of hydrocodone and acetaminophen from the drug coating. Subsequent to the initial release of hydrocodone and acetaminophen, the sustained release of the dosage forms provides for continued release of hydrocodone and acetaminophen to the patient, as demonstrated by the sustained hydroco done and acetaminophen plasma levels shown in FIGS. 9 and 10. The plasma concentrations of hydromorphone, a metabolite of hydrocodone, are shown in Tables 2 and 4 discussed above and FIG. 11.

For study Regimens E and F, steady state plasma concentrations are shown in Table 2 and FIGS. 14-17. These results demonstrate a decreased fluctuation in plasma hydrocodone and acetaminophen when patients were dosed with the controlled release formulations in comparison with every 4 hour dosing of an immediate release formulation of hydrocodone and acetaminophen. The results also demonstrate that for hydrocodone the peak concentration is in general less than twice as large as the minimum concentration and that for acetaminophen the peak concentration is in general less than 3.5 times as large as the minimum concentration.

Overall, in this clinical trial, the sustained release dosage forms of hydrocodone and acetaminophen concentrations were dose proportional across 1, 2 and 3 tablets.

Steady state for the sustained release dosage forms of hydrocodone and acetaminophen Q12H was achieved by 24 hours; no statistically significant monotonic rising time effect was observed in the hydrocodone and acetaminophen trough concentrations measured between 24 and 72 hours. Accumulation was minimal as steady-state peak concentrations of hydrocodone were less than 50% and concentrations of acetaminophen were less than 25% greater than those achieved following the administration of a single dose. Hydromorphone levels reached steady state during the second day of dosing as the 36 and 72 hours hydromorphone trough concentrations were not statistically significantly different.

The test Regimen B (single dose of the sustained release dosage forms of hydrocodone and acetaminophen, 2 tablets) was equivalent to reference Regimen D (NORCO®, 1 tablet every 4 hours for 3 doses) with respect to AUC; the 90% confidence intervals for the ratios of AUC central values for hydrocodone and acetaminophen were contained within the 0.80 to 1.25 range. The ratio of the Regimen B to Regimen D Cmax central values was estimated to be 0.79 for hydrocodone and 0.81 for acetaminophen, and both estimated ratios statistically lower than 1.0. The lower bound of the 90% confidence intervals for the ratios of hydrocodone and acetaminophen Cmax central values fell below 0.80.

The test Regimen E (the sustained release dosage forms of hydrocodone and acetaminophen, 2 tablets Q12H) was equivalent to the reference Regimen F (NORCO,® 1 tablet Q4H) at steady state; the 90% confidence intervals for the ratios of AUC and Cₘₐₓ central values for hydrocodone and acetaminophen were contained within the 0.80 to 1.25 range.

### EXAMPLE 7

An acute pain study was initiated to test the in vivo efficacy of dosage forms prepared as described in Example 2. The in vivo efficacy was tested in a third clinical trial of patients undergoing bunionectomy surgery. The study protocol and results are described below.

Methods:

Two hundred twelve volunteers undergoing bunionectomy surgery were enrolled in a randomized, double blind Phase II single and multiple-dose study. Subjects were to be given one of three dosage forms by oral administration, as follows:
(1) a controlled release HBH/APAP product prepared by the method described in Example 2 (one tablet totaling 15 mg HBH and 500 mg APAP), having a T₉₀ value of 8 hours and a matching placebo (one tablet) Q12H for 5 doses (Regimen 1);
(2) a controlled release HBH/APAP product prepared by the method described in Example 2 (two tablets totaling 30 mg HBH and 1000 mg APAP), having a T₉₀ value of 8 hours Q12H for 5 doses (Regimen 2); or
(3) two placebo tablets Q12H for 5 doses (Regimen 3).

Blood samples were collected from approximately half of the subjects for pharmacokinetic sampling at approximate times after administration as follows: 0, 1 hr, 2 hr, 4 hr, 8 hr, 48 hr and 60 hr. Blood samples were collected from the remaining subjects at approximately 0, 48 hr and 60 hr. Blood samples were processed to separate plasma for further analysis, and plasma concentrations of hydrocodone, acetaminophen and hydromorphone were determined. Analytical procedures were performed using a validated HPLC/MS/MS with quantitation between 0.092 and 92 ng/mL for hydrocodone, 5 and 10,000 ng/mL for acetaminophen and 0.1 and 100 ng/mL hydromorphone.

Efficacy assessments, including the categorical pain relief, meaningful and perceptible pain relief, pain intensity (categorical and visual analog scale) and subject global assessments, were completed by the subject and recorded. Measures of pain relief were calculated using the following definitions:
PR (Pain Relief): the pain relief at an evaluation;
TOTPAR (Total Pain Relief): the time interval weighted sum of pain relief;
PI (Pain Intensity): the observed pain intensity at an evaluation;
SPI (Sum of Pain Intensity): the time interval weighted sum of pain intensity.

The primary efficacy measurement was the TOTPAR score for 0 to 12 hours following the initial dose of study drug on Study Day 1. The TOTPAR score was a measure of the cumulative pain relief during treatment. One of the secondary measures was the SPI at the end of each dosing interval.

Results:

The pharmacokinetics of hydrocodone and acetaminophen were similar to those described in the pharmacokinetic study described above in Examples 5 and 6. Results are shown in Table 13

TABLE 13
Mean ± SD and Ranges for Pharmacokinetic Parameters

| | Hydrocodone | | Acetaminophen | |
|---|---|---|---|---|
| Single Dose | 1 Tablet | 2 Tablets | 1 Tablet | 2 Tablets |
| Cmax (ng/mL) | 12.2 ± 2.7 | 22.6 ± 6.0 | 1920 ± 533 | 3380 ± 675 |
| Range, ng/mL | 8.6-9.3 | 13.5-36.2 | 841-3195 | 1888-4715 |
| Tmax (h) | 5 ± 3.5 | 6 ± 3.8 | 2.3 ± 2.9 | 1.8 ± 1.5 |
| Steady State | 14.5 ± 4.7 | 29.7 ± 11.5 | 1130 ± 512 | 2070 ± 1010 |
| C48 (ng/mL) | | | | |
| Range, ng/mL | 5.3-8.1 | 1-50 | 370-3260 | 80-4829 |
| C60 (ng/mL) | 15.8 ± 5.6 | 29.6 ± 10.4 | 1320 ± 572 | 2430 ± 1060 |
| Range, ng/mL | 5.6-7.7 | 0.5-56 | 111-3030 | 111-5316 |

In the analysis of time interval sum of pain relief (TOTPAR) score during the 12-hour time interval after the initial dose of study drug, statistically significant differences were observed between Regimens 1 and 2 compared to Regimen 3, with higher mean TOTPAR scores (indicating better pain relief) in Regimens 1 and 2. In addition, a statistically significant difference was observed between Regimens 1 and 2, with better pain relief demonstrated in Regimen 2 than Regimen 1. The mean (standard error, SE) TOTPAR scores for the 0-12 hour time interval after the initial study drug administration are presented in Table 14.

TABLE 14
Analysis of Mean (SE) TOTPAR (0-12 hours) AUC Pain Scores
Following the Initial Study Drug Dose, Excluding Pain Assessments
After Rescue Medication Use (Intent-to-Treat Dataset)

| | Regimen 1 | Regimen 2 | Regimen 3 |
|---|---|---|---|
| Treatment | (N = 70) | (N = 70) | (N = 72) |
| TOTPAR^{a} | 6.4 (0.99)*^{†} | 13.3 (1.00)* | 2.2 (0.98) |

SE = standard error
*Statistically significant (p ≦ 0.05) difference versus Regimen 3, using a 2-way ANOVA with factors for treatment and investigator.
^{†}Statistically significant (p ≦ 0.05) difference versus Regimen 2, using a 2-way ANOVA with factors for treatment and investigator.
^{a}Least square means from 2-way ANOVA without interaction.

Sum of pain intensity (SPI) was assessed for each 12-hour period following each dose of study drug (i.e., five 12-hour post dose periods). Based on both the categorical and VAS scores, statistically significant differences were observed between Regimen 3 and Regimen 1 during the first 2 post dose periods and between Regimen 3 and Regimen 2 during all 5 periods, with lower mean scores (indicating less pain) in Regimens 1 and 2. A summary of the sum of pain intensity scores (categorical and VAS) following each of the 5 doses of study drug is presented in Table 15.

TABLE 15
Mean Pain Intensity Scores Following Each Dose
of Study Drug (Intent-to-Treat Dataset)

| | | | |
|---|---|---|---|
| | Regimen 1 | Regimen 2 | Regimen 3 |
| Pain Measure | (N = 70) | (N = 70) | (N = 72) |
| (Time Interval) | Mean (SE)^{b} | Mean (SE)^{b} | Mean (SE)^{b} |

SPI (Categorical)^{a}

| | | | | | | |
|---|---|---|---|---|---|---|
| Post dose 1 (0-12 hours) | 27.2 | (0.8)*^{†} | 22.7 | (0.8)* | 30.1 | (0.8) |
| Post dose 2 (0-12 hours) | 13.0 | (1.0)* | 11.2 | (1.1)* | 17.0 | (1.0) |
| Post dose 3 (0-12 hours) | 12.8 | (1.0)^{†} | 9.7 | (1.0)* | 14.7 | (1.0) |
| Post dose 4 (0-12 hours) | 10.6 | (1.0) | 8.0 | (1.0)* | 12.8 | (0.9) |
| Post dose 5 (0-12 hours) | 10.9 | (1.0)^{†} | 7.8 | (1.0)* | 11.8 | (1.0) |
| SPI (VAS)^{c} | | | | | | |
| Post dose 1 (0-12 hours) | 791.9 | (27.4)*^{†} | 614.8 | (27.5)* | 886.0 | (27.0) |
| Post dose 2 (0-12 hours) | 353.9 | (33.2)* | 266.3 | (33.3)* | 462.1 | (32.7) |
| Post dose 3 (0-12 hours) | 318.0 | (32.4)^{†} | 206.2 | (32.4)* | 379.6 | (31.9) |
| Post dose 4 (0-12 hours) | 254.6 | (28.6)^{†} | 155.3 | (28.7)* | 310.2 | (28.2) |
| Post dose 5 (0-12 hours) | 240.05 | (29.1)^{†} | 147.8 | (29.2)* | 285.4 | (28.6) |

SE = standard error
*Statistically significant (p ≦ 0.05) difference versus Regimen 3, using a 2-way ANOVA with factors for treatment and investigator.
^{†}Statistically significant (p ≦ 0.05) difference versus Regimen 2, using a 2-way ANOVA with factors for treatment and investigator.
^{a}Categorical Pain Intensity Score: 0 = none, 1 = mild, 2 = moderate, 3 = severe.
^{b}Least square means from 2-way ANOVA without interaction.
^{c}VAS Pain Intensity Scale: 0 to 100 (100-mm VAS).

This formulation showed excellent in vivo efficacy (pain relief) in a post-operative setting. In addition, as shown above and in Examples 5 and 6, this formulation provided effective plasma concentrations of hydrocodone bitartrate and acetaminophen over a 12-hour period, and exhibited decreased plasma fluctuations (peaks and valleys) than provided by a comparable immediate release formulation, thereby providing plasma concentrations of analgesic agents effective to provide pain relief that are relatively constant over time. Such constant and effective concentrations of analgesic agents provide the potential for greater pain relief when compared to a comparable dose of an immediate release formulation that does not maintain plasma concentrations of analgesic agents in a constant and effective range of plasma concentrations. In addition, such constant and effective concentrations of analgesic agents provide the potential for effective pain relief using a smaller amount of analgesic agents, and may further provide increased safety, in comparison with comparable immediate release analgesic formulation. Finally, there is the likelihood of greater patient compliance with the prescribed dosage regimen due to the consistent pain relief as well as the convenience of twice a day dosing.

### EXAMPLE 8

### Layered Matrix Tablets Providing Immediate Release and Sustained Release of 500 mg Acetaminophen (APAP) and 15 mg Hydrocodone Bitartrate (HB)

The layered matrix tablets consist of an immediate release (IR) layer, a sustained release (SR) APAP layer (SR APAP) and a sustained release HB layer (SR HB). The immediate release portion of the tablets consists of both APAP and HB. The blend was prepared by directly mixing the dry powders of APAP and HB with Prosolv SMCC 90 (silicified microcrystalline cellulose), lactose, Klucel EXF (hydroxypropyl cellulose, HPC), Crospovidone and magnesium stearate for 5 minutes prior to compression. The composition of the IR layer in a triple layer tablet is as follows:

| Ingredient | Amount per tablet (mg) |
|---|---|
| Acetaminophen (APAP) | 100 |
| Hydrocodone Bitartrate (HB) | 3 |
| ProSolv SMCC 90 | 70.9 |
| Klucel EXF | 7 |
| Lactose (Anhydrous) | 10 |
| Magnesium Stearate | 0.6 |
| Crospovidone | 2.5 |
| Total weight per tablet | 194 mg |

The SR APAP layer blend was also made by the dry blending approach. The blend was prepared by direct mixing of APAP with Prosolv SMCC 90, lactose, Klucel EXF, Ethocel FP 10 (ethylcellulose, EC), Eudragit EPO (aminoalkyl methacrylate copolymers), sodium dodecyl sulfate and magnesium stearate for 5 minutes. This was followed by slugging, grinding and passing through a 20 mesh screen before tableting.

The composition of the SR APAP layer in a triple layer matrix tablets is as follows:

| Ingredient | Amount per tablet (mg) |
|---|---|
| Acetaminophen (APAP) | 400 |
| ProSolv SMCC 90 | 68 |
| Klucel EXF | 23 |
| Lactose (Anhydrous) | 88 |
| Ethocel FP 10 | 10 |
| Eudragit E PO | 15 |
| Sodium Dodecyl Sulfate (SDS) | 5 |
| Magnesium Stearate | 2 |
| Total weight per tablet | 611 mg |

The SR HB blend was prepared by first melting Compritol 888 ATO (Glyceryl Behenate) at approximately 70° C. in a container. This was followed by adding HB, Prosolv SMCC 90 and lactose while maintaining mixing. Upon congealing at room temperature, the granulation was passed through a 20 mesh screen. Based on the yield, the amount of HPC and magnesium stearate was added and blended for 5 minutes.

The composition of the SR HB layer in a triple layer matrix tablets is as follows:

| Ingredient | Amount per tablet (mg) |
|---|---|
| Hydrocodone Bitartrate (HB) | 12 |
| ProSolv SMCC 90 | 136.4 |
| Klucel EXF | 10 |
| Lactose (Anhydrous) | 23 |
| Magnesium Stearate | 0.6 |
| Compritol 888 ATO | 80 |
| Total weight per tablet | 262 mg |

Following the preparation of the IR, SR APAP and SR HB blends, triple layer tablets were made on a Carver Press. In tableting, a 7/16 inch (1.09 mm) diameter flat face round tooling was used. The IR blend was loaded into the die cavity with light tamping applied; this was followed by adding the SR APAP blend and light tamping, and lastly the SR HB blend before final compression. Depending on the compression force used, different tablet hardness was obtained. The triple layer tablets used for the release assay were made under final compression force of ˜3900 Lbs (hardness ˜35 Strong-Cobb Units (SCU)).

Release assays were conducted in 900 ml of 0.01N HCl (pH ˜2) and pH 6.8 phosphate buffer solution at ˜37±0.5° C., respectively, by using USP apparatus II (Paddle method). A sinker was used. The paddle speed was set at 50 rpm and 10 ml sample was taken at each sampling point and analyzed by HPLC.

The release results are presented in Tables 16 and 17 below, comparing the layered matrix system with an osmotic dosage form (sample B in Example 4, above). The comparisons are based on the fact that the in vitro drug release of the osmotic dosage form is known to be independent of test media and conditions used.

The similarity between the release profiles was quantified using the similarity factor f₂ as proposed by Moore and Flanner [Pharmaceutical Technology, 20:64-74, 1996]. The f₂ value is a measure of the similarity between two release profiles and ranges from 0-100. As per FDA guideline [Guidance for Industry, 1997. Modified release solid dosage forms: scale-up and post-approval changes: Chemistry, manufacturing and controls, in vitro release testing, and in vivo bioequivalence documentation], drug release profiles are defined as similar when f₂ lies between 50 and 100. Such an analysis between the release profiles of the two types of systems yielded f₂ values of 60.8 and 67.5 for APAP and HB, respectively, in pH 6.8 phosphate buffer, and 44.1 and 82.6 for APAP and HB, respectively, in 0.01 N HCl. The slightly lower f₂ value of APAP in 0.01 N HCl was primarily due to faster release and higher amount of drug in the IR release portion when compared to the osmotic dosage form. Thus, similarity can be enhanced by varying the ratio of IR to SR of the matrix system, or the formulation composition (see Example 9 below).

TABLE 16
Cumulative release from a layered matrix tablet vs. osmotic
dosage form in pH 6.8 phosphate buffer (n = 3)

| | | | Osmotic dosage form (sample B | |
|---|---|---|---|---|
| | Layered matrix | | in Table 8 of Example 4) | |
| System | % APAP | | % APAP | |
| Time (hrs) | Released | % HB Released | Released | % HB Released |
| 0.5 | 19.6 | 23.8 | 13 | 24 |
| 1 | 27.8 | 30.7 | 36 | 36 |
| 3 | 52.7 | 48.3 | 49 | 51 |
| 5 | 69.0 | 68.9 | 66 | 73 |
| 6 | 74.9 | 79.7 | 76 | 83 |
| 7 | 80.0 | 87.9 | 89 | 95 |
| 8 | 84.2 | 93.8 | 99 | 102 |
| 10 | 90.8 | 100.7 | 102 | 105 |

TABLE 17
Layered matrix tablet vs. osmotic dosage form in 0.01 N HCl (n = 3)

| | | | Osmotic pump (from sample B in | |
|---|---|---|---|---|
| | Layered matrix | | Table 8 of Example 4, p91) | |
| System | % APAP | | % APAP | |
| Time (hrs) | Released | % HB Released | Released | % HB Released |
| 0.5 | 24.4 | 25.5 | 13 | 24 |
| 1 | 35.5 | 32.1 | 36 | 36 |
| 3 | 67.3 | 50.5 | 49 | 51 |
| 5 | 85.5 | 72.4 | 66 | 73 |
| 6 | 89.4 | 84.3 | 76 | 83 |
| 7 | 90.7 | 88.9 | 89 | 95 |
| 8 | 90.8 | 92.6 | 99 | 102 |
| 10 | 90.6 | 95.2 | 102 | 105 |

### EXAMPLE 9

The same type of design as described in Example 8 was used to prepare matrix tablets that provide immediate release and sustained release of 500 mg Acetaminophen (APAP) and 15 mg Hydrocodone Bitartrate (HB). The IR portion of the tablets consists of both APAP and HB. The blend was prepared by directly mixing the dry powders of APAP and HB with Avicel PH 102 (microcrystalline cellulose), lactose, Klucel EXF, and magnesium stearate for 5 minutes prior to compression. The composition of the IR layer in a triple layer matrix tablet is as follows:

| Ingredient | Amount per tablet (mg) |
|---|---|
| Acetaminophen (APAP) | 100 |
| Hydrocodone Bitartrate (HB) | 3 |
| Avicel PH 102 | 63.4 |
| Klucel EXF | 7 |
| Lactose (Anhydrous) | 20 |
| Magnesium Stearate | 0.6 |
| Total weight per tablet | 194 mg |

The SR APAP layer blend was also made by a dry blending approach. The blend was prepared by direct mixing of APAP with Avicel PH 102, lactose, Klucel EXF, Ethocel FP 10, Eudragit and magnesium stearate for 5 minutes. This was followed by slugging, grinding and passing through a 20 mesh screen before tableting. The composition of the SR APAP layer in a triple layer matrix tablet is as follows:

| Ingredient | Amount per tablet (mg) |
|---|---|
| Acetaminophen (APAP) | 400 |
| Avicel PH 102 | 78 |
| Klucel EXF | 23 |
| Lactose (Anhydrous) | 88 |
| Ethocel FP 10 | 10 |
| Eudragit E PO | 10 |
| Magnesium Stearate | 2 |
| Total weight per tablet | 611 mg |

The SR HB blend was prepared by first melting Compritol 888 ATO (Glyceryl Behenate) at approximately 70° C. in a container. This was followed by adding HB, Avicel PH 102 and lactose while maintaining mixing. Upon congealing at room temperature, the granulation was passed through a 20 mesh screen. Based on the yield, the amount of HPC and magnesium stearate was added and blended for 5 minutes. The composition of the SR HB layer in a triple layer matrix tablet is as follows:

| Ingredient | Amount per tablet (mg) | |
|---|---|---|
| Hydrocodone Bitartrate (HB) | 12 | |
| Avicel PH 102 | 124.4 | |
| Klucel EXF | 10 | |
| Lactose (Anhydrous) | 23 | |
| Magnesium Stearate | 0.6 | |
| Compritol 888 ATO | 92 | |
| Total weight per tablet | 262 | mg |

Following the preparation of the IR, SR APAP and SR HB blends, triple layer tablets were made on a Carver Press. In tableting, a 7/16 inch (1.09 mm) diameter flat face round tooling was used. The IR blend was loaded into the die cavity with light tamping applied; this was followed by adding the SR APAP blend and light tamping, and lastly the SR HB blend before final compression. The compression force used in making these tablets was 2500 lbs.

The same method as that described in Example 8 was used to test release rates of both actives from the matrix tablet in 0.01N HCl (pH ˜2) and pH 6.8 phosphate buffer, respectively. The similarity factor (f₂) was calculated using the release profile of the osmotic dosage form (from sample B in Table 8 of Example 4) as reference. Only one data point of >80% release was used in the calculation. The test results are listed in the Table 18 demonstrating that release of both APAP and HB from the matrix tablet is similar to that of osmotic dosage form (sample B in Example 4) as defined by f₂ values.

TABLE 18
Release data from a triple layer dosage form in 0.01
N HCl and pH 6.8 phosphate buffer (n = 3).

| | % APAP | % APAP | % HB | % HB |
|---|---|---|---|---|
| Time | Released | Released | Released | Released |
| (hrs) | (pH 6.8) | (pH 2) | (pH 6.8) | (pH 2) |
| 0.5 | 22.5 | 24.8 | 24.7 | 24.2 |
| 1 | 30.3 | 34.8 | 32.3 | 32.2 |
| 3 | 52.2 | 59.5 | 56.6 | 54.0 |
| 5 | 66.6 | 75.8 | 76.6 | 72.4 |
| 6 | 71.8 | 81.7 | 83.2 | 81.0 |
| 7 | 76.2 | 84.9 | 88.0 | 87.2 |
| 8 | 80.2 | 86.2 | 90.6 | 91.9 |
| 10 | 86.4 | 87.9 | 93.7 | 98.0 |
| f₂ | 50.4 | 54.3 | 72.5 | 79.6 |

### EXAMPLE 10

During the study of Example 8, it was observed that tablet hardness increased on storage after compression. To study the effect of this change on release rate, a release study of the freshly prepared and the same batch of tablets stored at the ambient temperature in a capped glass bottle for 3 days was performed under the same release conditions as described in Example 8. The results indicate that the release rate remains essentially unchanged despite increased tablet hardness upon storage. The tablet hardness and release data of this study is presented below.

TABLE 19
Effect of storage/hardness change on release (n = 3)

| | % APAP | % APAP | % HB | % HB |
|---|---|---|---|---|
| Time | Released | Released | Released | Released |
| (hrs) | (Fresh-35 Scu) | (3 days 50 Scu) | (Fresh-35 Scu) | (3 days 50 Scu) |
| 0.5 | 24.4 | 26.0 | 25.5 | 28.6 |
| 1 | 35.5 | 38.3 | 32.1 | 35.5 |
| 3 | 67.3 | 69.0 | 50.5 | 52.6 |
| 5 | 85.5 | 84.3 | 72.4 | 70.2 |
| 6 | 89.4 | 89.6 | 84.3 | 81.2 |
| 7 | 90.7 | 92.9 | 88.9 | 89.6 |
| 8 | 90.8 | 93.5 | 92.6 | 94.5 |
| 10 | 90.6 | 93.7 | 95.2 | 98.6 |

### EXAMPLE 11

To study the impact of compression force on the release rates of the triple layer matrix tablets presented in Example 8, two compression forces were used to prepare tablets using the same blend. Tablets were tested under the same release conditions described in Example 8 except that pH 6.8 0.05 M phosphate buffer was used to release media. Results indicated that within the range investigated, the release rates of APAP could be altered by adjusting the compression force while the release rate of HB was insensitive to compression force. The release data are listed in the following table.

TABLE 20
Effect of compression force on release (n = 3)

| | % APAP | | | |
|---|---|---|---|---|
| | Released | % APAP | % HB | % HB Released |
| Time | 4000 lb | Released | Released | 3000 lb |
| (hrs) | (36 scu) | 3000 lb (32 scu) | 4000 lb (36 scu) | (32 scu) |
| 0.5 | 29.2 | 42.1 | 30.7 | 31.5 |
| 1 | 37.1 | 58.7 | 36.2 | 36.8 |
| 3 | 63.1 | 84.8 | 55.0 | 59.3 |
| 5 | 80.7 | 92.3 | 79.2 | 82.8 |
| 6 | 84.4 | 92.7 | 87.1 | 89.6 |
| 7 | 87.8 | 92.2 | 93.3 | 93.9 |
| 8 | 89.4 | 92.1 | 96.6 | 96.6 |
| 10 | 91.6 | 91.8 | 100.5 | 99.6 |

### EXAMPLE 12

The release rate of APAP and HB in triple layer matrix tablets can also be altered by varying the composition in each layer. A new formulation was made using the same manufacturing procedure and tested under the same release conditions as described in the Example 8. The results indicated that different release profiles can be obtained by adjusting the formulation composition. The triple layer matrix formulation composition is as follows:

| | IR layer | SR APAP layer | SR HB layer |
|---|---|---|---|
| Excipients | (mg) | (mg) | (mg) |
| Acetaminophen, (APAP) | 100 | 400 | |
| Hydrocodone Bitartrate (HB) | 3 | | 12 |
| Avicel PH 102 | 79.4 | 73 | 99.4 |
| Klucel EXF | 7 | 23 | 10 |
| Lactose (Anhydrous) | 4 | 88 | 20 |
| Magnesium Stearate | 0.6 | 2 | 0.6 |
| Ethocel FP 10 | | 10 | |
| Eudragit EPO | | 10 | |
| Compritol 888 ATO | | | 120 |
| Sodium Dodecyl Sulfate | | 5 | |
| Total weight in each triple | 194 | 611 | 262 |
| layer tablet (mg) | | | |

TABLE 21
Release data of triple layer matrix tablets in Example 8
and the tablets made from the above table (n = 2)

| | % APAP | % APAP | | % HB |
|---|---|---|---|---|
| Time | Released | Released | % HB Released | Released |
| (hrs) | 3900 Lb | 6000 Lb | 3900 Lb | 6000 Lb |
| 0.5 | 24.4 | 14.6 | 25.5 | 15.0 |
| 1 | 35.5 | 20.9 | 32.1 | 20.5 |
| 3 | 67.3 | 40.0 | 50.5 | 34.5 |
| 5 | 85.5 | 54.3 | 72.4 | 43.2 |
| 6 | 89.4 | 60.4 | 84.3 | 46.8 |
| 7 | 90.7 | 66.0 | 88.9 | 50.1 |
| 8 | 90.8 | 71.0 | 92.6 | 53.0 |
| 10 | 90.5 | 78.4 | 95.3 | 59.9 |

### EXAMPLE 13

### Multi-Unit Dosage Form that Provides Immediate Release and Sustained Release of 500 mg Acetaminophen and 15 mg Hydrocodone Bitartrate

The multiple units in this type of dosage form may exist as small tablets, pellets or beads with size ranging from micrometers to millimeters. The multi-unit dosage form tested consists of three types of tablets encapsulated into a single capsule. The three types of small tablets are IR tablets, SR APAP matrix tablets and SR HB matrix tablets.

The immediate release tablets consist of both APAP and HB. Dry blending and direct compression were used in the preparation of the tablets. The blend was prepared by mixing APAP and HB dry powders with Avicel PH 102, lactose, Klucel EXF, sodium starch glycolate and magnesium stearate for 2 minutes prior to compression. The composition of the IR tablets is as follows:

| Ingredient | Amount per tablet (mg) | |
|---|---|---|
| Acetaminophen (APAP) | 50 | |
| Hydrocodone Bitartrate (HB) | 1.5 | |
| Avicel PH 102 | 30 | |
| Klucel EXF | 5 | |
| Lactose (Anhydrous) | 30 | |
| Magnesium Stearate | 0.5 | |
| Sodium starch glycolate | 3 | |
| Total weight per tablet | 120 | mg |

The SR APAP tablets blend was prepared by first melting Compritol 888 ATO at approximately 70° C. in a container. This is followed by adding APAP, Avicel PH 102, lactose, EUDRAGIT EPO, and sodium dodecyl sulfate (SDS) while maintaining mixing. Upon congealing at room temperature, the granulation was passed through a 20 mesh screen. Based on the yield, the amount of HPC and magnesium stearate is added and mixed for another 5 minutes prior to compression. The composition of the SR APAP matrix tablets is as follows:

| Ingredient | Amount per tablet (mg) | |
|---|---|---|
| Acetaminophen (APAP) | 90 | |
| Avicel PH 102 | 15.4 | |
| Klucel EXF | 5 | |
| Lactose (Anhydrous) | 11 | |
| EUDRAGIT EPO | 5 | |
| Compritol 888 ATO | 13 | |
| Sodium Dodecyl Sulfate (SDS) | 0.2 | |
| Magnesium Stearate | 0.4 | |
| Total weight per tablet | 140 | mg |

The SR HB blend was prepared by first melting Compritol 888 ATO at approximately 70° C. in a container, this was followed by adding HB, Prosolv SMCC 90and lactose while maintaining mixing. Upon congealing at room temperature, the granulation was passed through a 20 mesh screen. Based on the yield, the amount of Klucel EXF and magnesium stearate was added and mixed for 5 minutes prior to compression. The composition of the SR HB tablets is as follows:

| Ingredient | Amount per tablet (mg) | |
|---|---|---|
| Hydrocodone Bitartrate (HB) | 6.75 | |
| ProSolv SMCC 90 | 73.3 | |
| Klucel EXF | 5.6 | |
| Lactose (Anhydrous) | 13 | |
| Magnesium Stearate | 0.35 | |
| Compritol 888 ATO | 49 | |
| Total weight per tablet | 148 | mg |

Following the preparation of the IR, SR APAP and SR HB blends, tablets were made on a Carver Press using a 9/32 inch (0.703 mm) diameter round concave tooling. The weights of IR, SR APAP and SR HB tablets were 120 mg, 140 mg and 148 mg, respectively. The IR tablet (1 tablet) contains 10% of the total HB and APAP unit dose; the SR APAP tablets (5 tablets) contain 90% of the total APAP unit dose; and the SR HB tablets (2 tablets) contains also 90% of the total HB unit dose. Prior to release study, 1 IR tablet, 5 SR APAP tablets and 2 SR HB tablets were filled into a capsule.

Following encapsulation, release tests were performed. Release tests were conducted by using USP apparatus II (Paddle method) with 900 ml of 0.01N HCl (pH ˜2) at ˜37±0.5° C. The paddle speed was set at 50 rpm and 10 ml sample was taken at each sampling point and analyzed by HPLC. Sinkers were not used in the release test. The release data of the multi-unit dosage forms are presented in the following table. The hardness of each type of unit was as follows: IR ˜8.1 SCU; SR HB ˜6.4 SCU, SR APAP ˜5.6 SCU.

TABLE 22
Release data of the multi-unit dosage forms (n = 3).

| Time (hrs) | % APAP Released | % HB Released |
|---|---|---|
| 0.5 | 21.5 | 18.2 |
| 1 | 32.1 | 27.1 |
| 3 | 59.9 | 51.0 |
| 5 | 78.8 | 68.3 |
| 6 | 85.0 | 75.2 |
| 7 | 89.2 | 84.2 |
| 8 | 91.7 | 90.2 |
| 10 | 93.5 | 96.6 |

### EXAMPLE 14

To study the effect of pH of release media on the release of the tablets presented in Example 12, the same batch of tablets were tested under the same release conditions presented in Example 12 in either 0.01N HCl (pH ˜2) or 0.05 M phosphate buffer (pH 6.8 PBS). The results indicate that the release rate of HB is essentially independent of pH while the release rate of APAP is generally not affected by pH for more than 50% of drug release. The release data is listed in the following table.

TABLE 23
Release data of multi-unit dosage form in 0.01
N HCl and pH 6.8 phosphate buffer (n = 3).

| | % APAP | % APAP | % HB | % HB |
|---|---|---|---|---|
| Time | Released | Released | Released | Released |
| (hrs) | (pH 6.8) | (pH 2) | (pH 6.8) | (pH 2) |
| 0.5 | 18.3 | 21.5 | 19.2 | 18.2 |
| 1 | 28.5 | 32.1 | 27.7 | 27.1 |
| 3 | 52.6 | 59.9 | 51.2 | 51.0 |
| 5 | 65.9 | 78.8 | 68.2 | 68.3 |
| 6 | 70.4 | 85.0 | 77.2 | 75.2 |
| 7 | 73.9 | 89.2 | 84.7 | 84.2 |
| 8 | 77.6 | 91.7 | 90.7 | 90.2 |
| 10 | 83.8 | 93.5 | 97.7 | 96.6 |

### EXAMPLE 15

### Compression Coated Tablets that Provide Immediate Release and Sustained Release of 500 mg Acetaminophen and 15 mg Hydrocodone Bitartrate

The compression coated tablets consist of a sustained release core tablet encased in an immediate release outer layer prepared by compression. The SR core is a bilayer tablet that contains an SR APAP layer and a SR HB layer. The compression coated layer is an immediate release formulation that contains both APAP and HB.

The IR blend: The immediate release blend was prepared by dry mixing APAP and HB with Avicel PH 102, lactose, Klucel EXF and magnesium stearate for 5 minutes. The composition of the IR Compression layer is as follows:

| Ingredient | Amount per tablet (mg) | |
|---|---|---|
| Acetaminophen (APAP) | 100 | |
| Hydrocodone Bitartrate (HB) | 3 | |
| Avicel PH 102 | 379.4 | |
| Klucel EXF | 7 | |
| Lactose (Anhydrous) | 4 | |
| Magnesium Stearate | 0.6 | |
| Total Weight Per Tablet | 494 | mg |

The SR APAP blend: the same blend was used as described in Example 8.

The SR HB blend: the same blend was used as described in Example 8.

Preparation of compression coated tablets consists of two steps. First, a bilayer core tablet was made by using 7/16 inch (10.9 mm) diameter flat face round tooling. This was carried out by adding 611 mg of the SR APAP blend to the die cavity with light tamping followed by adding 262 mg of the SR HB blend before compression into tablet. The compression force used was 6000 lbs. In the second step, the compression coated tablet was made by using a 9/16 inch (14.1 mm) diameter round concave tooling. This was done by loading ˜25% of IR blend followed by placing the bilayer core tablet (prepared in the first step) in the center of the die cavity and finally adding the remaining 75% IR blend before compression. The total weight of the compression coated layer is 494 mg per tablet. The compression force used was 1000 lbs.

Release tests of the compression coated tablets were conducted using USP apparatus II (Paddle method) with 900 ml of 0.01N HCl (pH ˜2) at ˜37±0.5° C. The paddle speed was set at 50 rpm and 10 ml sample was taken at predetermined sampling point and analyzed by HPLC. Sinkers were used in the release test. The release data of the compression coated tablets are listed in the following table.

TABLE 24
Release data of Compression coated tablets in 0.01 N HCl (n = 3).

| Time (hrs) | % APAP Released | % HB Released |
|---|---|---|
| 0.5 | 17.5 | 16.7 |
| 1 | 35.2 | 24.7 |
| 3 | 73.2 | 52.1 |
| 5 | 81.6 | 75.0 |
| 6 | 82.8 | 81.9 |
| 7 | 83.3 | 86.0 |
| 8 | 83.6 | 88.5 |
| 10 | 84.6 | 91.2 |

### EXAMPLE 16

### Multi-Unit Dosage Form that Provides Immediate Release and Sustained Release of 500 mg Acetaminophen

The multiple units in this type of dosage form may exist as small tablets, pellets or beads with size ranging from micrometers to millimeters. To obtain a commercial dosage form, the small units can either be filled into a capsule by mixing IR and SR units. The small SR units may also be blended with excipients and IR portion of the actives followed by compressing into a disintegrating tablet. Alternatively, the IR portion can be coated onto the SR portion.

The multi-unit dosage form prepared consists of two types of small tablets. Those units can be encapsulated if needed. The two types of small units are IR APAP tablets and SR APAP tablets. Unlike the SR APAP tablets presented in Example 12, a sustained release film coating of ethylcellulose was applied to an APAP core tablet to obtain a SR APAP tablet.

Direct compression was used in the preparation of the IR tablets. The blend was prepared by mixing APAP with Avicel PH 102, lactose, sodium starch glycolate for 3 min; this was followed by adding magnesium stearate and mixing for an additional 3 minutes. Following the preparation of the IR APAP blend, tablets were made on a Carver Press using a 9/32 inch (0.703 mm) diameter round concave tooling. The weight of IR APAP tablet was 200 mg. The compression force used in the preparation these tablets was 1000 lbs. The composition of the IR tablets is as follows:

| Ingredient | Amount per tablet (mg) | |
|---|---|---|
| Acetaminophen (APAP) | 150 | |
| Avicel PH 102 | 22.5 | |
| Lactose (Anhydrous) | 22.5 | |
| Magnesium Stearate | 1 | |
| Sodium Starch Glycolate | 4 | |
| Total weight per tablet | 200 | mg |

The SR APAP core tablet was also prepared by direct compression. The blend was prepared by mixing APAP with Avicel PH 102 and lactose for 3 min; this was followed by adding magnesium stearate and mixing for an additional 3 minutes. Tablets were made on a Carver Press using 9/32 inch (0.703 mm) diameter round concave tooling. The weights of the tablets were 140 mg. The compression force used in the preparation of these tablets was 600 lbs. The IR APAP tablet and SR APAP core tablets contain 30% and 70% of total APAP amount, respectively.

The SR APAP core tablets were coated using film coating of ethylcellulose to achieve sustained release. The coating solution contains ethylcellulose (Ethocel 7FP), Klucel EXF, triethyl citrate and acetone. The composition of the coating solution is listed in the table below. The coating solution was prepared by adding Ethocel 7FP, Klucel EXF and triethyl citrate to acetone while maintaining agitation until all solids are in solution. The coating was carried out by applying a thin film to the tablets via iterations of dipping and drying cycles until a target weight gain was obtained. The weight gain for the tablets was 3.1%. The composition of the SR APAP core tablet is as follows:

| Ingredient | Amount per tablet (mg) | |
|---|---|---|
| Acetaminophen (APAP) | 87.5 | |
| Avicel PH 102 | 9.5 | |
| Lactose (Anhydrous) | 42.25 | |
| Magnesium Stearate | 0.75 | |
| Total weight per tablet | 140 | mg |

The composition of coating solution is as follows:

| Ingredient | Amount per batch (g) | |
|---|---|---|
| Ethocel 7FP | 9 | |
| Klucel EXF | 6 | |
| Triethyl Citrate | 3 | |
| Acetone | 232 | |
| Total weight of the coating solution | 250 | g |

Following the preparation of the IR APAP and SR APAP tablets. A combination of one IR APAP tablet and four SR APAP tablets were tested in the release study. Release was performed using USP apparatus II (Paddle method) with 900 ml of 0.01N HCl (pH ˜2) at ˜37±0.5° C. The paddle speed was set at 50 rpm and 5 ml sample was taken at each sampling point and analyzed by UV. A sinker was not used in the release test. The IR tablet (1 tablet) contains 30% of the total APAP unit dose; the SR APAP tablets (4 tablets) contain 70% of the total APAP unit dose. The release data of the multi-unit dosage forms is shown in Table 25 below.

TABLE 25
Release data of multi-unit dosage form in 0.01 N HCl (n = 3).

| Time (hrs) | % APAP Released |
|---|---|
| 0.33 | 39.5 |
| 0.75 | 45.7 |
| 1 | 50.0 |
| 3 | 75.3 |
| 5 | 84.1 |
| 6 | 91.1 |
| 7 | 96.8 |
| 8 | 99.9 |

### EXAMPLE 17

### Multi-Unit Dosage Form that Provides Immediate Release and Sustained Release of 500 mg Acetaminophen

The multiple units in this type of dosage form may exist as small tablets, pellets or beads with size ranging from micrometers to millimeters. To obtain a commercial dosage form, the small units can be filled into a capsule by mixing IR and SR units. The small SR units may also be mixed with excipients and IR portion of the actives and subsequently compressed into a disintegrating tablet. Alternatively, the IR portion can be coated onto the SR portion.

By using the same dosage form design presented in Example 16, the release profile of APAP can be tailored by varying the loading of APAP in IR tablet, SR APAP tablets and the amount of sustained release coating. In this study, a different ratio of IR to SR of APAP (compared to Example 16) was used. The same tablet preparation procedure and testing method as illustrated in Example 16 were used. Different from the APAR IR formulation presented in Example 16, only 10% of total APAP was used in the IR APAP tablets in this example.

The compression forces used in making IR APAP and SR APAP tablets were 1000 lbs and 3000 lbs, respectively. The same coating solution and coating procedure were applied to prepare SR APAP tablets. Alternatively, the IR portion can be coated onto the SR portion. The coating weight gain was 2.9%. The drug release was tested using the same method described in Example 16. The release data of these tablets is presented below in Table 26.

The composition of the APAP IR tablets is as follows:

| Ingredient | Amount per tablet (mg) | |
|---|---|---|
| Acetaminophen (APAP) | 50 | |
| Avicel PH 102 | 22.5 | |
| Lactose (Anhydrous) | 22.5 | |
| Magnesium Stearate | 1 | |
| Sodium Starch Glycolate | 4 | |
| Total weight per tablet | 100 | mg |

The composition of the SR APAP tablets is as follows:

| Ingredient | Amount per tablet (mg) | |
|---|---|---|
| Acetaminophen (APAP) | 112.5 | |
| Avicel PH 102 | 9.5 | |
| Lactose (Anhydrous) | 42.25 | |
| Klucel EXF | 5 | |
| Magnesium Stearate | 0.75 | |
| Total weight per tablet (uncoated) | 170 | mg |

TABLE 26
Release data of the multi-unit dosage forms (n = 3)

| Time (hrs) | % APAP Released |
|---|---|
| 0.33 | 14.2 |
| 0.75 | 16.5 |
| 1 | 18.3 |
| 3 | 33.5 |
| 5 | 47.9 |
| 6 | 56.3 |
| 7 | 65.3 |
| 8 | 72.8 |
| 9 | 79.8 |
| 12 | 93.3 |

### EXAMPLE 18

### Multi-Unit Dosage Form that Provides Immediate Release and Sustained Release of 15 mg Hydrocodone Bitartrate

A multi-unit dosage form that provides immediate release and sustained release of HB was made in this study. The multiple units in this type of dosage form may exist as small tablets, pellets or beads with size ranging from micrometers to millimeters. To obtain a commercial dosage form, the small units can either be filled into a capsule by mixing IR and SR units. The small SR units may also be mixed with excipients and IR portion of the actives and subsequently compressed into a disintegrating tablet. Alternatively, the IR portion can be coated onto the SR portion.

The same tablet preparation procedure and test method as illustrated in Example 16 were used. The compression force of IR HB and SR HBH core tablets were 300 lbs and 600 lbs, respectively. The IR HB tablet and SR HB core tablets contain 30% and 70% of total HB amount, respectively. The same coating solution and coating procedure as described in Example 16 were applied to prepare SR HB tablets. The coating weight gain was 20%. Each unit dose consists of one IR HB tablet and one SR HB tablet. Release samples were analyzed by HPLC in this study and the data of these tablets were listed below.

The composition of the IR HB tablets is as follows:

| Ingredient | Amount per tablet (mg) | |
|---|---|---|
| Hydrocodone Bitartrate (HB) | 4.5 | |
| Avicel PH 102 | 18.5 | |
| Lactose (Anhydrous) | 60 | |
| Magnesium Stearate | 1 | |
| Klucel EXF | 4 | |
| Sodium Starch Glycolate | 2 | |
| Total weight per tablet | 90 | mg |

The composition of the SR HB core tablets is as follows:

| Ingredient | Amount per tablet (mg) | |
|---|---|---|
| Hydrocodone Bitartrate (HB) | 10.5 | |
| Avicel PH 102 | 14 | |
| Lactose (Anhydrous) | 109.8 | |
| Magnesium Stearate | 0.7 | |
| Total weight per tablet | 135 | mg |

The drug release was tested using the same method described in Example 16. The release data of the multi-unit dosage forms is presented in Table 27 below:

TABLE 27
Release data of the multi-unit dosage forms (n = 4).

| Time (hrs) | % APAP Released |
|---|---|
| 0.5 | 34.7 |
| 1 | 37.4 |
| 3 | 78.8 |
| 5 | 96.0 |
| 6 | 101.0 |
| 7 | 103.6 |
| 8 | 104.5 |
| 10 | 105.5 |

### EXAMPLE 19

### Multi-Unit Dosage Form that Provides Immediate Release (IR) and Sustained Release (SR) of 500 mg Acetaminophen and 15 mg Hydrocodone Bitartrate

The multiple units in this type of dosage form may exist as small tablets, pellets or beads with size ranging from micrometers to millimeters. To obtain a commercial dosage form, the small units can either be filled into a capsule by mixing IR and SR units. The small SR units may also be mixed with excipients and IR portion of the actives and subsequently compressed into a disintegrating tablet. Alternatively, the IR portion can be coated onto the SR portion.

The multi-unit dosage form that provides IR and SR of Acetaminophen and Hydrocodone Bitartrate can be prepared by simply combining tablets of Example 16 or Example 17 with those of Example 18. More specifically, the dosage form can be obtained by encapsulating three types of small tablets into a single capsule: (1) IR tablets, (2) SR APAP tablets and (3) SR HB tablets. The same formulations and procedures described in Examples 16 and 18 can be used for preparation of the IR tablets, SR APAP tablets and SR HB tablets, respectively.

As an example, the following combinations can be tested in a release assay:
One IR tablet containing 30% of the total APAP unit dose;
One IR tablet containing 30% of the total HB unit dose;
Four SR APAP tablets containing 70% of the total APAP unit dose;
One SR HB tablet containing 70% of the total HB unit dose.

Following encapsulation of the tablets, a release test can be performed using the procedure described in Examples 16 or 18. Because there are no known interactions between the drugs released from each type of tablets, drug release from each tablet will be independent of each other. Thus, one can expect to obtain drug release profiles of APAP and HB that will be a result of superposition of the individual APAP and HB profiles given in Examples 16 and 18 if one would perform such a study. The dosage form can be further simplified by incorporating IR APAP and IR HB into one single tablet using an approach similar to that described in Example 13.

### EXAMPLE 20

### Layered Matrix Tablets that Provide Immediate Release and Sustained Release of 500 mg Acetaminophen and 7.5 mg Hydrocodone Bitartrate

In this example, the formulation design is the same as that in Example 8 except that a combination of 7.5 mg HB and 500 mg APAP were used in the triple layer tablet.

The immediate release portion of the tablets consists of both APAP and HB. The blend was prepared by mixing APAP and HB with Prosolv SMCC 90, lactose, Klucel EXF, sodium starch glycolate and magnesium stearate for 5 minutes prior to compression. The composition of the IR layer in a triple layer tablet is as follows:

| Ingredient | Amount per tablet (mg) | |
|---|---|---|
| Acetaminophen (APAP) | 100 | |
| Hydrocodone Bitartrate (HB) | 1.5 | |
| ProSolv SMCC 90 | 70.9 | |
| Klucel EXF | 7 | |
| Lactose (Anhydrous) | 11.5 | |
| Sodium Starch Glycolate | 2.5 | |
| Magnesium Stearate | 0.6 | |
| Total weight per tablet | 194 | mg |

The SR APAP layer was prepared by directly mixing of APAP with Prosolv SMCC 90, lactose, Klucel EXF, Ethocel FP 10, Eudragit E PO, and magnesium stearate for 5 minutes. The composition of the SR APAP layer in a triple layer tablets is as follows:

| Ingredient | Amount per tablet (mg) | |
|---|---|---|
| Acetaminophen (APAP) | 400 | |
| ProSolv SMCC 90 | 68 | |
| Klucel EXF | 23 | |
| Lactose (Anhydrous) | 88 | |
| Ethocel FP 10 | 10 | |
| Eudragit E PO | 20 | |
| Magnesium Stearate | 2 | |
| Total weight per tablet | 611 | mg |

The SR HB blend was prepared by first melting Compritol 888 ATO at approximately 70° C. in a container. This was followed by adding HB, Prosolv SMCC 90 and lactose while maintaining mixing. Upon congealing at room temperature, the granulation was passed through a 20 mesh screen. Based on the yield, the amount of Klucel EXF and magnesium stearate was added and blended for 5 minutes. The composition of the SR HB layer in a triple layer tablets is as follows:

| Ingredient | Amount per tablet (mg) | |
|---|---|---|
| Hydrocodone Bitartrate (HB) | 6 | |
| ProSolv SMCC 90 | 136.4 | |
| Klucel EXF | 10 | |
| Lactose (Anhydrous) | 29 | |
| Magnesium Stearate | 0.6 | |
| Compritol 888 ATO | 80 | |
| Total weight per tablet | 262 | mg |

The same procedures for tablet preparation and release method were used as those described in Example 16. The final compression force used was 4200 lbs. The release of the triple layer matrix tablet is presented in Table 28 below.

TABLE 28
Release data for the triple layer tablet.

| Time (hrs) | % APAP Released | % HB Released |
|---|---|---|
| 0.5 | 25.0 | 40.2 |
| 1 | 32.7 | 48.2 |
| 3 | 51.4 | 69.1 |
| 5 | 66.8 | 86.5 |
| 6 | 73.3 | 90.7 |
| 7 | 78.1 | 92.9 |
| 8 | 81.7 | 94.2 |
| 10 | 87.6 | 94.5 |

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

1. A sustained release dosage form for twice daily oral dosing to a human patient, comprising
a) an immediate release component;
b) a sustained release component,
wherein said immediate release component and said sustained release component collectively contain a therapeutically effective amount of an opioid analgesic and a therapeutically effective amount of nonopioid analgesic, wherein said amount of nonopioid analgesic is between about 20 and about 100 times said amount of opioid analgesic by weight, and said sustained release component provides sustained release of each of said opioid analgesic and said nonopioid analgesic at rates proportional to each other in said dosage form.

2. The sustained release dosage form of claim 1, wherein said nonopioid analgesic has a solubility of less than about 10 mg/mL at 25° C.

3. The sustained release dosage form of claim 1, wherein said amount of nonopioid analgesic is between about 20 and about 40 times said amount of opioid analgesic by weight.

4. The sustained release dosage form of claim 3, wherein said amount of nonopioid analgesic is between about 27 and about 34 times said amount of opioid analgesic by weight.

5. The sustained release dosage form of claim 1, wherein the nonopioid analgesic is acetaminophen and the opioid analgesic is hydrocodone bitartrate.

6. The sustained release dosage form of claim 5, wherein the sustained release component contains a loading of acetaminophen of at least 60% by weight.

7. The sustained release dosage form of claim 6, wherein the sustained release component contains a loading of acetaminophen of between about 75% and about 95% by weight.

8. The sustained release dosage form of claim 5, wherein when administered to the human patient, the dosage form produces a plasma profile characterized by a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose.

9. The sustained release dosage form of claim 5, wherein when administered to the human patient, the dosage form produces a minimum Cmax for hydrocodone of about 0.4 ng/mL/mg to a maximum Cmax for hydrocodone of about 1.9 ng/mL/mg and a minimum Cmax for acetaminophen of about 2.0 ng/mL/mg and maximum Cmax for acetaminophen of about 10.4 ng/mL/mg after a single dose.

10. The sustained release dosage form of claim 5, wherein when administered to the human patient, the dosage form produces a Cmax for hydrocodone of about 0.8±0.2 ng/mL/mg and a Cmax for acetaminophen of about 4.1±1.1 ng/mL/mg after a single dose.

11. The sustained release dosage form of claim 5, wherein when administered to the human patient, the dosage form produces a Tmax for hydrocodone of about 1.9±2.1 to about 6.7±3.8 hours after a single dose.

12. The sustained release dosage form of claim 5, wherein when administered to the human patient, the dosage form produces a Tmax for hydrocodone of about 6.7±3.8 hours after a single dose.

13. The sustained release dosage form of claim 5, wherein when administered to the human patient, the dosage form produces a Tmax for hydrocodone of about 4.3±3.4 hours after a single dose.

14. The sustained release dosage form of claim 5, wherein when administered to the human patient, the dosage form produces a Tmax for acetaminophen of about 1.1±1.1 to about 2.8±2.7 hours after a single dose.

15. The sustained release dosage form of claim 5, wherein when administered to the human patient, the dosage form produces a Tmax for acetaminophen of about 1.1±1.1 hours after a single dose.

16. The sustained release dosage form of claim 5, wherein when administered to the human patient, the dosage form produces an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

17. The sustained release dosage form of claim 5, wherein when administered to the human patient, the dosage form produces a minimum AUC for hydrocodone of about 7.0 ng*hr/mL/mg to a maximum AUC for hydrocodone of about 26.2 ng*hr/mL/mg and a minimum AUC for acetaminophen of about 18.4 ng*hr/mL/mg and maximum AUC for acetaminophen of 79.9 ng*hr/mL/mg after a single dose.

18. The sustained release dosage form of claim 5, wherein when administered to the human patient, the dosage form produces an AUC for hydrocodone of about 15.0±3.7 ng*hr/mL/mg and an AUC for acetaminophen of 41.1±12.4 ng*hr/mL/mg after a single dose.

19. The sustained release dosage form of claim 5, wherein when administered to the human patient, the dosage form produces a Cmax for hydrocodone of between about 0.6 ng/mL/mg to about 1.4 ng/mL/mg and a Cmax for acetaminophen of between about 2.8 ng/mL/mg and 7.9 ng/mL/mg, and wherein the dosage form produces an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

20. The sustained release dosage form of claim 5, wherein when administered to the human patient, the dosage form produces a Cmax for hydrocodone of between about 19.4 and 42.8 ng/ml after a single dose of 30 mg hydrocodone.

21. The sustained release dosage form of claim 5, wherein when administered to the human patient, the dosage form produces a minimum Cmax for hydrocodone of about 12.7 ng/ml and the maximum Cmax for hydrocodone of about 56.9 ng/mL after a single dose of 30 mg hydrocodone.

22. The sustained release dosage form of claim 5, wherein when administered to the human patient, the dosage form produces a Cmax for hydrocodone of between about 25.3±5.7 ng/ml after a single dose of 30 mg hydrocodone.

23. The sustained release dosage form of claim 5, wherein when administered to the human patient, the dosage form produces a Cmax for acetaminophen of between about 3.0 and about 7.9 μg/ml after a single dose of 1000 mg acetaminophen.

24. The sustained release dosage form of claim 5, wherein when administered to the human patient, the dosage form produces a minimum Cmax for acetaminophen of about 2.0 μg/ml and the maximum Cmax of about 10.4 μg/ml after a single dose of 1000 mg acetaminophen.

25. The sustained release dosage form of claim 5, wherein when administered to the human patient, the dosage form produces a Cmax for acetaminophen of between about 4.1±1.1 μg/ml after a single dose of 1000 mg acetaminophen.

26. The sustained release dosage form of claim 5, wherein when administered to the human patient, the plasma concentration profile for hydrocodone exhibits an area under the concentration time curve between about 275 and about 562 ng*hr/ml after a single dose of 30 mg hydrocodone bitartrate.

27. The sustained release dosage form of claim 5, wherein when administered to the human patient, the plasma concentration profile for hydrocodone exhibits a minimum area under the concentration time curve of about 228 ng*hr/ml and a maximum area under the concentration time curve of about 754 ng*hr/ml after a single dose of 30 mg hydrocodone bitartrate.

28. The sustained release dosage form of claim 5, wherein when administered to the human patient, the plasma concentration profile for hydrocodone exhibits an area under the concentration time curve of about 449±113 ng*hr/ml after a single dose of 30 mg hydrocodone bitartrate.

29. The sustained release dosage form of claim 5, wherein when administered to the human patient, the plasma concentration profile for acetaminophen exhibits an area under the concentration time curve between about 28.7 and about 57.1 μg*hr/ml after a single dose of 1000 mg acetaminophen.

30. The sustained release dosage form of claim 5, wherein when administered to the human patient, the plasma concentration profile for acetaminophen exhibits a minimum area under the concentration time curve of about 22.5 μg*hr/ml and a maximum area under the concentration time curve of about 72.2 μg*hr/ml after a single dose of 1000 mg acetaminophen.

31. The sustained release dosage form of claim 5, wherein when administered to the human patient, the plasma concentration profile for acetaminophen exhibits an area under the concentration time curve of about 41.1±12.4 μg*hr/ml after a single dose of 1000 mg acetaminophen.

32. The sustained release dosage form of claim 5, wherein when administered to the human patient, the dosage form produces a Cmax for hydromorphone of between about 0.12 and about 0.35 ng/ml after a single dose of 30 mg hydrocodone to a non-poor CYP2D6 metabolizer human patient.

33. The sustained release dosage form of claim 5, wherein when administered to the human patient, the plasma concentration for hydrocodone at 12 hours (C12) is between about 11.0 and about 27.4 ng/ml after a single dose of 30 mg hydrocodone bitartrate in a human patient.

34. The sustained release dosage form of claim 5, wherein when administered 5 to the human patient, the plasma concentration for acetaminophen at 12 hours (C12) is between about 0.7 and 2.5 μg/ml after a single dose of 1000 mg acetaminophen in a human patient.

35. The sustained release dosage form of claim 5, wherein when administered to the human patient, the plasma concentration profile exhibits a width at half height value for hydrocodone of between about 6.4 and about 19.6 hours.

36. The sustained release dosage form of claim 5, wherein when administered to the human patient, the plasma concentration profile exhibits a width at half height value for acetaminophen of between about 0.8 and about 12.3 hours.

37. The sustained release dosage form of claim 5, wherein when administered to the human patient, the plasma concentration profile exhibits a weight ratio of acetaminophen to hydrocodone between about 114.2 and 284 at one hour after oral administration of a single dose containing 1000 mg acetaminophen and 30 mg hydrocodone to a human patient.

38. The sustained release dosage form of claim 5, wherein when administered to the human patient, the plasma concentration profile exhibits a weight ratio of acetaminophen to hydrocodone between about 70.8 and 165.8 at six hours after oral administration of a single dose containing 1000 mg acetaminophen and 30 mg hydrocodone to a human patient.

39. The sustained release dosage form of claim 5, wherein when administered to the human patient, the plasma concentration profile exhibits a weight ratio of acetaminophen to hydrocodone between about 36.4 and 135.1 at 12 hours after oral administration of a single dose containing 1000 mg acetaminophen and 30 mg hydrocodone to a human patient.

40. The sustained release dosage form of claim 5, wherein the dosage form produces a plasma concentration profile for hydrocodone characterized by a first peak concentration (Cmax1) occurring within about 1 to 2 hours after oral administration and a second peak concentration (Cmax2), occurring from about 5 to about 9 hours after oral administration to the human patient.

41. The sustained release dosage form of claim 5, wherein the dosage form produces a plasma concentration profile for acetaminophen characterized by a first peak concentration (Cmax1) occurring within about 1 hour after oral administration and a second peak concentration (Cmax2), occurring from about 4 to about 8 hours after oral administration to the human patient.

42. The sustained release dosage form of claim 5, wherein the dosage form produces a plasma concentration profile for hydrocodone characterized by a first peak concentration occurring at a time Tmax1 occurring from about 0.4 to about 2.5 hours after oral administration and a second peak concentration occurring at a time Tmax2 occurring from about 2.9 to about 11.4 hours after oral administration to the human patient.

43. The sustained release dosage form of claim 5, wherein the dosage form produces a plasma concentration profile for hydrocodone characterized by a first peak concentration occurring at a time Tmax1 occurring at about 1.6±0.9 hours after oral administration and a second peak concentration occurring at a time Tmax2 occurring at about 9.0±2.4 hours after oral administration to the human patient.

44. The sustained release dosage form of claim 5, wherein the dosage form produces a plasma concentration profile for acetaminophen characterized by a first peak concentration occurring at a time Tmax1 occurring within about 0.5 to about 1.8 hours after oral administration and a second peak concentration occurring at a time Tmax2 occurring from about 1.7 to about 11.9 hours after oral administration to the human patient.

45. The sustained release dosage form of claim 5, wherein the dosage form produces a plasma concentration profile for acetaminophen characterized by a first peak concentration occurring at a time Tmax1 occurring within about 0.7±0.2 hours after oral administration and a second peak concentration occurring at a time Tmax2 occurring from about 7.7±4.2 hours after oral administration to the human patient.

46. The sustained release dosage form of claim 5, wherein the dosage form produces a plasma concentration profile for hydrocodone further characterized by a minimum concentration (Cmin) between Cmax1 and Cmax2 after oral administration to the human patient.

47. The sustained release dosage form of claim 46, wherein the Cmax1 for hydrocodone is from about 15.8 ng/mL to about 35.4 ng/mL.

48. The sustained release dosage form of claim 46, wherein the minimum Cmax1 for hydrocodone is about 5.4 ng/mL and the maximum Cmax1 is about 41.7 ng/mL.

49. The sustained release dosage form of claim 46, wherein the Cmax2 for hydrocodone is from about 16.2 ng/mL to about 40.5 ng/mL.

50. The sustained release dosage form of claim 46, wherein the minimum Cmax2 for hydrocodone is about 12.7 ng/mL and the maximum Cmax2 is about 56.9 ng/mL.

51. The sustained release dosage form of claim 46, wherein the Cmin for hydrocodone is from about 10.1 ng/mL to about 23.5 ng/mL.

52. The sustained release dosage form of claim 46, wherein the minimum Cmin 30 for hydrocodone is about 5.2 ng/mL and the maximum Cmin is about 30.9 ng/mL.

53. The sustained release dosage form of claim 46, wherein the dosage form produces a plasma concentration profile for acetaminophen further characterized by a minimum concentration (Cmin) between Cmax1 and Cmax2 after oral administration to the human patient.

54. The sustained release dosage form of claim 53, wherein the Cmax1 for acetaminophen is from about 2.9 μg/mL to about 7.9 μg/mL.

55. The sustained release dosage form of claim 53, wherein the minimum Cmax1 for acetaminophen is about 1.6 μg/mL and the maximum Cmax1 is about 10.4 μg/mL.

56. The sustained release dosage form of claim 53, wherein the Cmax2 for acetaminophen is from about 1.5 μg/mL to about 5.6 μg/mL.

57. The sustained release dosage form of claim 53, wherein the minimum Cmax2 for acetaminophen is about 1.0 μg/mL and the maximum Cmax2 is about 8.8 μg/mL.

58. The sustained release dosage form of claim 53, wherein the Cmin for acetaminophen is from about 1.2 μg/mL to about 3.8 μg/mL.

59. The sustained release dosage form of claim 53, wherein the minimum Cmin for acetaminophen is about 0.7 μg/mL and the maximum Cmin is about 4.5 μg/mL.

60. The sustained release dosage form of claim 1, wherein the sustained release component comprises:
(1) a semipermeable wall defining a cavity and including an exit orifice formed or formable therein;
(2) a drug layer comprising a therapeutically effective amount of an opioid analgesic and a nonopioid analgesic contained within the cavity and located adjacent to the exit orifice;
(3) a push displacement layer contained within the cavity and located distal from the exit orifice;
(4) a flow-promoting layer between the inner surface of the semipermeable wall and at least the external surface of the drug layer that is opposite the wall;
wherein the dosage form provides an in vitro rate of release of the opioid analgesic and the nonopioid analgesic for up to about 12 hours after being contacted with water in the environment of use.

61. The sustained release dosage form of claim 60, wherein the drug layer contains a loading of the nonopioid analgesic of at least 60% by weight.

62. The sustained release dosage form of claim 61, wherein the drug layer contains a loading of the nonopioid analgesic of between about 75% and about 95% by weight.

63. The sustained release dosage form of claim 62, wherein the drug layer contains a loading of the nonopioid analgesic of between about 80% and about 85% by weight.

64. The sustained release dosage form of claim 60, wherein the drug layer contains a loading of the opioid analgesic between about 1% and about 10% by weight.

65. The sustained release dosage form of claim 64, wherein the drug layer contains a loading of the opioid analgesic between about 2% and about 6% by weight.

66. The sustained release dosage form of claim 60, wherein the amount of the nonopioid analgesic is between about 27 and about 34 times the amount of the opioid analgesic by weight.

67. The sustained release dosage form of claim 60, wherein the drug layer is exposed to the environment of use as an erodible composition.

68. The sustained release dosage form of claim 60, wherein the in vitro rate of release of the opioid analgesic and the nonopioid analgesic is zero order or ascending.

69. The sustained release dosage form of claim 60, wherein the in vitro rate of release of the opioid analgesic and nonopioid analgesic is maintained for from about 6 hours to about 10 hours.

70. The sustained release dosage form of claim 69, wherein the in vitro rate of release of the opioid analgesic and nonopioid analgesic is maintained for about 8 hours.

71. The sustained release dosage form of claim 60, wherein the drug layer further comprises a disintegrant, a surfactant, a binding agent, or a gelling agent, or mixtures thereof.

72. The sustained release dosage form of claim 71, wherein the drug layer further comprises a nonionic surfactant.

73. The sustained release dosage form of claim 71, wherein the nonionic surfactant is a poloxamer, or a fatty acid ester of polyoxyethylene, or mixtures thereof.

74. The sustained release dosage form of claim 60, wherein the opioid analgesic is selected from hydrocodone, hydromorphone, oxymorphone, methadone, morphine, codeine, or oxycodone, or pharmaceutically acceptable salts thereof.

75. The sustained release dosage form of claim 60, wherein the nonopioid analgesic is acetaminophen.

76. The sustained release dosage form of claim 60, wherein the nonopioid analgesic is acetaminophen and the opioid analgesic is hydrocodone bitartrate.

77. The sustained release dosage form of claim 1, wherein the immediate release component comprises a drug coating comprising a therapeutically effective amount of an opioid analgesic and nonopioid analgesic sufficient to provide an analgesic effect in a patient in need thereof.

78. The sustained release dosage form of claim 76, wherein the immediate release component comprises a drug coating comprising a therapeutically effective amount of an hydrocodone bitartrate and acetaminophen sufficient to provide an analgesic effect in a patient in need thereof.

79. The sustained release dosage form of claim 78, wherein the drug coating comprises from about 60% to about 96.99% acetaminophen by weight.

80. The sustained release dosage form of claim 79, wherein the drug coating comprises from about 75% to about 89.5% acetaminophen by weight.

81. The sustained release dosage form of claim 78, wherein the drug coating comprises from about 0.01% to about 25% hydrocodone bitartrate by weight.

82. The sustained release dosage form of claim 81, wherein the drug coating comprises from about 0.5% to about 15% hydrocodone bitartrate by weight.

83. The sustained release dosage form of claim 78, wherein the drug coating comprises from about 1% to about 3% hydrocodone bitartrate by weight.

84. The sustained release dosage form of claim 78, wherein the dosage form exhibits a release rate in vitro of the opioid analgesic and nonopioid analgesic of from about 19% to about 49% released after 0.75 hour, from about 40% to about 70% released after 3 hours, and at least about 80% released after 6 hours.

85. The sustained release dosage form of claim 78, wherein the dosage form exhibits a release rate in vitro of the opioid analgesic and nonopioid analgesic of from about 19% to about 49% released after 0.75 hour, from about 35% to about 65% released after 3 hours, and at least about 80% released after 8 hours.

86. The sustained release dosage form of claim 78, wherein the dosage form exhibits a release rate in vitro of the opioid analgesic and nonopioid analgesic of from about 19% to about 49% released after 0.75 hour, from about 35% to about 65% released after 4 hours, and at least about 80% released after 10 hours.

87. The sustained release dosage form of claim 60, wherein at least 90% of the nonopioid analgesic and at least 90% of the opioid analgesic are released from the dosage form within 12 hours of being contacted with water in the environment of use.

88. A sustained release dosage form for twice daily oral dosing to a human patient, comprising
a) an immediate release component;
b) a sustained release component,
wherein said immediate release component and said sustained release component collectively contain a therapeutically effective amount of hydrocodone and a therapeutically effective amount of acetaminophen, wherein said amount of acetaminophen is between about 20 and about 100 times said amount of hydrocodone by weight, and wherein the dosage form produces a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose.

89. The sustained release dosage form of claim 88, wherein said sustained release component provides sustained release of each of said hydrocodone and said acetaminophen at rates proportional to each other.

90. A sustained release dosage form for twice daily oral dosing to a human patient, comprising
a) an immediate release component;
b) a sustained release component, wherein said immediate release component and said sustained release component collectively contain a therapeutically effective amount of hydrocodone and a therapeutically effective amount of acetaminophen, wherein said amount of acetaminophen is between about 20 and about 100 times said amount of hydrocodone by weight, and wherein the dosage form produces an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

91. The sustained release dosage form of claim 90, wherein said sustained release component provides sustained release of each of said hydrocodone and said acetaminophen at rates proportional to each other.

92. A sustained release dosage form for twice daily oral dosing to a human patient, comprising
a) an immediate release component;
b) a sustained release component,
wherein said immediate release component and said sustained release component collectively contain a therapeutically effective amount of hydrocodone and a therapeutically effective amount of acetaminophen, wherein said amount of acetaminophen is between about 20 and about 100 times said amount of hydrocodone by weight, wherein the dosage form exhibits a plasma concentration profile for hydrocodone characterized by a first peak concentration (Cmax1) occurring within about 1 to 2 hours after oral administration and a second peak concentration (Cmax2), occurring from about 5 to about 9 hours after oral administration to the human patient.

93. A sustained release dosage form for twice daily oral dosing to a human patient, comprising
an immediate release component; and
a sustained release component,
wherein said immediate release component and said sustained release component collectively provide a therapeutically effective amount of a nonopioid analgesic and an opioid analgesic, and wherein said immediate release component and said sustained release compoenent provide a means for providing a Cmax for hydrocodone of between about 0.6 ng/mL/mg to about 1.4 ng/mL/mg and a Cmax for acetaminophen of between about 2.8 ng/mL/mg and 7.9 ng/mL/mg after a single dose in the plasma of the patient.

94. A sustained release dosage form for twice daily oral dosing to a human patient, comprising
an immediate release component; and
a sustained release component,
wherein said immediate release component and said sustained release component collectively provide a therapeutically effective amount of a nonopioid analgesic and an opioid analgesic, and wherein said immediate release component and said sustained release compoenent provide a means for providing an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

95. A method for treating pain in a human patient, comprising orally administering to the human patient on a twice-a-day basis an oral sustained release dosage form comprising:
(1) a semipermeable wall defining a cavity and including an exit orifice formed or formable therein;
(2) a drug layer comprising a therapeutically effective amount of an opioid analgesic and a nonopioid analgesic contained within the cavity and located adjacent to the exit orifice;
(3) a push displacement layer contained within the cavity and located distal from the exit orifice;
(4) a flow-promoting layer between the inner surface of the semipermeable wall and at least the external surface of the drug layer that is opposite the wall;
wherein the dosage form provides an in vitro rate of release of the opioid analgesic and the nonopioid analgesic for up to about 12 hours after being contacted with water in the environment of use.

96. The method of claim 95, wherein the drug layer contains a loading of the nonopioid analgesic of at least 60% by weight.

97. The method of claim 96, wherein the drug layer contains a loading of the nonopioid analgesic of between about 75% and about 95% by weight.

98. The method of claim 97, wherein the drug layer contains a loading of the nonopioid analgesic of between about 80% and about 85% by weight.

99. The method of claim 95, wherein the drug layer contains a loading of the opioid analgesic between about 1% and about 10% by weight.

100. The method of claim 96, wherein the drug layer contains a loading of the opioid analgesic between about 2% and about 6% by weight.

101. The method of claim 95, wherein the amount of the nonopioid analgesic is between about 20 and about 100 times the amount of the opioid analgesic by weight.

102. The method of claim 101, wherein the amount of the nonopioid analgesic is between about 20 and about 40 times the amount of the opioid analgesic by weight.

103. The method of claim 102, wherein the amount of the nonopioid analgesic is between about 27 and about 34 times the amount of the opioid analgesic by weight.

104. The method of claim 95, wherein the dosage form releases the opioid analgesic and the nonopioid analgesic at proportional rates relative to each other.

105. The method of claim 95, wherein the drug layer is exposed to the environment of use as an erodible composition.

106. The method of claim 95, wherein the in vitro rate of release of the opioid analgesic and the nonopioid analgesic is zero order or ascending.

107. The method of claim 95, wherein the in vitro rate of release of the opioid analgesic and nonopioid analgesic is maintained for from about 6 hours to about 10 hours.

108. The method of claim 95, wherein the in vitro rate of release of the opioid analgesic and nonopioid analgesic is maintained for about 8 hours.

109. The method of claim 95, wherein said opioid analgesic is hydrocodone and pharmaceutically acceptable salts thereof.

110. The method of claim 95, wherein said nonopioid analgesic is acetaminophen.

111. The method of claim 95, further comprising a drug coating comprising an effective amount of an immediate release analgesic composition located on the outside surface of the at least partially semipermeable wall.

112. The method of claim 111, wherein the dosage form produces a plasma profile characterized by a Cmax for hydrocodone of between about 0.6 ng/mL/mg to about 1.4 ng/mL/mg and a Cmax for acetaminophen of between about 2.8 μg/mL/mg and 7.9 μg/mL/mg in the human patient after a single dose.

113. The method of claim 111, wherein the dosage form produces a plasma profile characterized by an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.

114. A method for providing an effective amount of an analgesic composition for treating pain in a human patient in need thereof, comprising orally admitting into a patient in need thereof a high load dosage form comprising an effective dose of an opioid analgesic agent and a nonopioid analgesic agent contained in a drug layer and an osmotic push composition, wherein said drug layer and push compositions are surrounded by an at least partially semipermeable wall permeable to the passage of water and impermeable to the passage of said analgesic agents, and an exit means in the wall for delivering the analgesic composition from the dosage form, wherein in operation, water enters through the at least partially semipermeable wall into the dosage form causing the osmotic push composition to expand and push the drug layer through the exit means, wherein the drug layer is exposed to the environment of use as an erodible composition, and wherein the nonopioid analgesic and the opioid analgesic are delivered at a controlled rate over a sustained period of time up to about 12 hours providing a therapeutically effective dose to the patient in need thereof.

115. The method of claim 114, wherein the dosage form is administered on a twice daily basis for the treatment of pain.

116. The method of claim 114, wherein the analgesic composition further comprises a drug coating providing an immediate delivery of an effective dose of an opioid analgesic agent and a nonopioid analgesic agent to a patient in need thereof.

117. A method for providing an effective concentration of an opioid analgesic and nonopioid analgesic in the plasma of a human patient for the treatment of pain, comprising orally admitting into a patient in need thereof a high load dosage form comprising an effective dose of an opioid analgesic agent and a nonopioid analgesic agent contained in a drug layer, an osmotic displacement composition, wherein said drug layer and displacement compositions are surrounded by an at least partially semipermeable wall permeable to the passage of water and impermeable to the passage of said analgesic agents, and an exit means in the wall for delivering the analgesic composition from the dosage form, wherein in operation, water enters through the at least partially semipermeable wall into the dosage form causing the osmotic displacement composition to expand and push the drug layer through the exit means, wherein the drug layer is exposed to the environment of use as an erodible composition, and wherein the nonopioid analgesic and the opioid analgesic are delivered at a proportional rate over a sustained period of time up to about 12 hours.

118. The method of claim 117, wherein the analgesic composition further comprises a drug coating providing an immediate delivery of an effective dose of an opioid analgesic agent and a nonopioid analgesic agent to a patient in need thereof.

119. The method of claim 118, wherein said opioid analgesic is hydrocodone and pharmaceutically acceptable salts thereof, and wherein said nonopioid analgesic is acetaminophen.

120. The method of claim 119, wherein the dosage form produces a plasma profile characterized by a Cmax for hydrocodone of between about 0.6 ng/mL/mg to about 1.4 ng/mL/mg and a Cmax for acetaminophen of between about 2.8 ng/mL/mg and 7.9 ng/mL/mg in the human patient after a single dose.

121. The method of claim 119, wherein the dosage form produces a plasma profile characterized by an AUC for hydrocodone of between about 9.1 ng*hr/mL/mg to about 19.9 ng*hr/mL/mg and an AUC for acetaminophen of between about 28.6 ng*hr/mL/mg and about 59.1 ng*hr/mL/mg after a single dose.
